SEQUENCE LISTING (5 sequences)

SEQ ID NO: 1 - DNA, 1534 bp, Pseudomonas fluorescens
tgaagagtttgatcatggctcagattgaacgctggcggcaggcctaacacatgcaagtcgagcggcagcacgggtacttgtacctggtggcgagcggcggacgggtgagtaatgcctaggaatctgcctagtagtgggggataacgtccggaaacgggcgctaataccgcatacgtcctacgggagaaagtgggggatcttcggacctcacgctattagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagttacctaatacgtgattgttttgacgttaccgacagaataagcaccggctaactctgtgccagcagccgcggtaatacagagggtgcaagcgttaatcggaattactgggcgtaaagcgcgcgtaggtggtttgttaagttggatgtgaaagccccgggctcaacctgggaactgcatccaaaactggcaagctagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcgggaggacggttaccacggtgtgattcatgactggggtgaagtcgtaacaaggtagccgtaggggaacctgcggctggatcacctcctt

SEQ ID NO: 2 - DNA, 1547 bp, Pseudomonas putida
gaactgaagagtttgatcatggctcagattgaacgctggcggcaggcctaacacatgcaagtcgagcggatgacgggagcttgctccttgattcagcggcggacgggtgagtaatgcctaggaatctgcctggtagtgggggacaacgtttcgaaaggaacgctaataccgcatacgtcctacgggagaaagcaggggaccttcgggccttgcgctatcagatgagcctaggtcggattagctagttggtggggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccgacagaataagcaccggctaactctgtgccagcagccgcggtaatacagagggtgcaagcgttaatcggaattactgggcgtaaagcgcgcgtaggtggtttgttaagttggatgtgaaagccccgggctcaacctgggaactgcatccaaaactggcaagctagagtacggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttggaatccttgagattttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatgcagagaactttccagagatggattggtgccttcgggaactctgacacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatctcacaaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcggggggacggttaccacggtgtgattcatgactggggtgaagtcgtaacaaggtagccgtaggggaacctgcggctggatcacctccttaatcgacgaca

SEQ ID NO: 3 - DNA, 1539 bp, Pseudomonas stutzeri
gaactgaagagtttgatcatggctcagattgaacgctggcggcaggcctaacacatgcaagtcgagcggatgagtggagcttgctccatgattcagcggcggacgggtgagtaatgcctaggaatctgcctggtagtgggggacaacgtttcgaaaggaacgctaataccgcatacgtcctacgggagaaagcaggggaccttcgggccttgcgctatcagatgagcctaggtcggattagctagttggtgaggtaaaggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccgacagaataagcaccggctaacttcgtgccagcagccgcggtaatacgaagggtgcaagcgttaatcggaattactgggcgtaaagcgcgcgtaggtggttcgttaagttggatgtgaaagccccgggctcaacctgggaactgcatccaaaactggcgagctagagtatggcagagggtggtggaatttcctgtgtagcggtgaaatgcgtagatataggaaggaacaccagtggcgaaggcgaccacctgggctaatactgacactgaggtgcgaaagcgtggggagcaaacaggattagataccctggtagtccacgccgtaaacgatgtcgactagccgttgggatccttgagatcttagtggcgcagctaacgcattaagtcgaccgcctggggagtacggccgcaaggttaaaactcaaatgaattgacgggggcccgcacaagcggtggagcatgtggtttaattcgaagcaacgcgaagaaccttaccaggccttgacatgcagagaactttccagagatggattggtgccttcgggaactctgacacaggtgctgcatggctgtcgtcagctcgtgtcgtgagatgttgggttaagtcccgtaacgagcgcaacccttgtccttagttaccagcacgttaaggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgctccagaagtagctagtctaaccttcggggggacggttaccacggagtgattcatgactggggtgaagtcgtaacaaggtagccgtaggggaacctgcggctggatcacctccttaat

SEQ ID NO: 4 - DNA, 1546 bp, Pseudomonas sp.
aactgaagagtttgatcatggctcagattgaacgctggcggcaggcctaacacatgcaagtcgagcggtagagagaagcttgcttctcttgagagcggcggacgggtgagtaatgcctaggaatctgcctagtggtgggggataacgttcggaaacggacgctaataccgcatacgtcctacgggagaaagcgggggaccttcgggcctcgcgccattagatgagcctaggtcggattagctagttggtgaggtaaaggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggtagtaacttaatacgttgctactttgacgttaccgacagaataagcaccggctaacttcgtgccagcagccgcggtaatacgaagggtgcaagcgttaatcggaattactgggcgtaaagcgcgcgtaggtggttcagtaagttggaagtgaaatccccgggctcaacctgggaactgctttcaaaactgctgagctagagtacggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttgggagtcttgaactcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttacctggccttgacatgctgagaactttctagagatagattggtgccttcgggaactcagacacaggtgctgcatggctgtcgtcagctcgtgtcgtgagatgttgggttaagtcccgtaacgagcgcaacccttgtccttagttaccagcacgttatggtgggaactctaaggagactgccggtgacaaaccggaggaaggtggggatgacgtcaagtcatcatggcccttacggccagggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgcaccagaagtagctagtctaaccgcaagggggacggttaccacggtgtgattcatgactggggtgaagtcgtaacaaggtagccgtaggggaacctgcggctggatcacctccttaatcgaagact

SEQ ID NO: 5 - DNA, 1523 bp, Rhodococcus sp.
ttcaacggagagtttgatcctggctcaggacgaacgctggcggcgtgcttaacacatgcaagtcgagcggtaaggcctttcggggtacacgagcggcgaacgggtgagtaacacgtgggtgatctgccctgcacttcgggataagcctgggaaactgggtctaataccggatatgacctcctatcgcatggtgggtggtggaaagatttatcggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgcaagtgacggtacctgcagaagaagcaccggctaactacgtgccagcagccgcggtaatacgtagggtgcaagcgttgtccggaattactgggcgtaaagagttcgtaggcggtttgtcgcgtcgtttgtgaaaaccagcagctcaactgctggcttgcaggcgatacgggcagacttgagtactgcaggggagactggaattcctggtgtagcggtgaaatgcgcagatatcaggaggaacaccggtggcgaaggcgggtctctgggcagtaactgacgctgaggaacgaaagcgtgggtagcgaacaggattagataccctggtagtccacgccgtaaacggtgggcgctaggtgtgggttccttccacggaatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagctgcagagatgtggccccccttgtggtcggtatacaggtggtgcatggctgtcgtcagctcgtgtcgtgagatgttgggttaagtcccgcaacgagcgcaacccctatcttatgttgccagcacgttatggtggggactcgtaagagactgccggggtcaactcggaggaaggtggggacgacgtcaagtcatcatgccccttatgtccagggcttcacacatgctacaatggccagtacagagggctgcgagaccgtgaggtggagcgaatcccttaaagctggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcttaaccccttgtgggagggagccgtcgaaggtgggatcggcgattgggacgaagtcgtaacaaggtagccgtaccggaaggtgcggctggatcacctcctttctaa

(19) United States
(12) Patent Application Publication
(10) Pub. No.: US 2020/0318163 A1
(43) Pub. Date: Oct. 8, 2020

(54) BIODEGRADATION OF TOXIC ORGANIC COMPOUNDS IN CONTAMINATED ENVIRONMENTS

(71) Applicant: Metabolik Technologies Inc., Vancouver (CA)

(72) Inventors: Parisa Chegounian, Vancouver (CA); Vikramaditya Ganapati Yadav, Vancouver (CA)

(73) Assignee: Metabolik Technologies Inc., Vancouver (CA)

(21) Appl. No.: 16/833,406

(22) Filed: Mar. 27, 2020

Related U.S. Application Data
(60) Provisional application No. 62/826,753, filed on Mar. 29, 2019.

(51) Int. Cl. C12Q 1/689; G16B 5/00; G16B 25/10; C12N 15/10; C12Q 1/6874

(52) U.S. Cl. CPC C12Q 1/689; G16B 5/00; G16B 25/10; C12Q 2600/158; C12Q 1/6874; C12Q 2600/142; C12N 15/1096

(57) ABSTRACT

The present disclosure relates generally to methods and/or means for generating and analyzing gene expression profiles of a microorganism isolated from an environment contaminated with toxic organic compounds. In particular, the disclosure relates to methods and/or means of identifying genes, enzymes, and metabolic pathways involved in naphthenic acids compounds (NAFC) or naphthenic acid (NA) degradation activity. Engineered microorganisms with increased naphthenic acid (NA) degradation activity and their use for biodegradation of toxic organic compounds in contaminated environments are further provided.

### CROSS-REFERENCE TO RELATED APPLICATIONS

This application claims priority to, and the benefit of, U.S. Provisional Patent Application Ser. No. 62/826,753 filed on Mar. 29, 2019 and entitled “BIODEGRADATION OF TOXIC ORGANIC COMPOUNDS IN CONTAMINATED ENVIRONMENTS,” the disclosure of which is incorporated herein by reference in its entirety.

### TECHNICAL FIELD

The present disclosure relates to biodegradation of toxic and environmental pollutants. In particular, it relates to methods of identifying and analyzing genes, enzymes, and metabolic pathways involved in adaptation to and/or degradation of naphthenic acids. The disclosure also relates to engineered microorganisms and their uses to increase naphthenic acids fraction compounds (NAFC) or naphthenic acid (NA) degradation activity for such biodegradation, which are useful in the treatment of contaminated environments (e.g., oil sands process-affected water).

### BACKGROUND

The extraction process of bitumen from oils sands produces process-affected water (e.g., oil sands process-affected water (OSPW)) as a by-product. The OSPW is contaminated with toxic organic compounds¹⁻⁴, and many oil sand companies presently operate on zero-discharge policy for OSPW. As a result, the OSPW is stored on-site in tailing ponds, typically for several decades⁵. Presently, about 732 billion litres OSPW sit in tailings ponds that span about 77 square kilometers in the Athabasca region of Alberta, Canada⁶. Consequently, there is an urgent need to treat this contaminated water so that it can eventually be returned to the environment in a re-useable form⁷⁻⁹.

OSPW contains several major classes of contaminants including, such as for example, naphthenic acids (NAs), polycyclic aromatic hydrocarbons (PAHs), BTEX (benzene, toluene, ethyl benzene, and xylenes), phenols, heavy metals and ions¹⁰. Classic naphthenic acids (NAs), the primary acute toxic constituent of OSPW^{2,4,11}, have long been defined as alkyl-substituted acyclic and cycloaliphatic carboxylic acids.

Current technologies in OSPW remediation include advanced oxidation/biodegradation, biodegradation/adsorption and flocculation/membrane filtration have been developed to achieve more efficient NA removal and sustainable process¹⁹. However, due to the high recalcitrancy^{20,21} of NA compounds to oxidation/biodegradation, incompatibility of physical treatment processes due to tailing ponds conditions¹⁹, and high costs associated with these technologies, current strategies do not provide a viable, safe, cost-effective, and sustainable approach to remediation. More specifically, NAs with greater carbon numbers and DBEs have been shown to be more recalcitrant and more toxic, due to their increased hydrophobicity and greater contribution to toxicity by narcosis²².

Technologies using naturally occurring microorganism for contaminant elimination have been around for some time. Early work by Herman et al.²⁴ first demonstrated the biodegradation of NAs by microorganisms. Further, with developments in recombinant DNA techniques, there have been attempts to apply this technology to the breakdown of recalcitrant pollutants³⁰ but these attempts have been met largely with failure. A lack of understanding and tools to investigate important parameters pertaining to microorganisms and their biodegradation processes has prevented further development of engineered microorganisms for such a purpose.

Consequently, there remains a need for such understanding and tools to provide for development of water treatment technologies to provide safer, effective, economically viable, and/or sustainable bioremediation strategies.

### SUMMARY

This Summary is provided to introduce a selection of concepts in a simplified form that are further described below in the Detailed Description. This Summary is not intended to identify key aspects or essential aspects of the claimed subject-matter.

As embodied and broadly described herein, the present disclosure relates to a method of generating gene expression profiles of a microorganism isolated from an environment contaminated with toxic organic compounds, comprising: preparing RNA samples from a first microorganism strain grown i) in the presence of naphthenic acids fraction compounds (NAFC) or naphthenic acids (NA) and ii) in the absence of naphthenic acids fraction compounds (NAFC) or naphthenic acids (NA); preparing amplicon cDNA libraries of differentially expressed genes of the RNA samples from i) and ii); determining the relative frequency of expression of RNA for the microorganism by sequencing the amplicon cDNA libraries; constructing an in silico genome assembly from the cDNA libraries; annotating the in silico genome assembly to predict differentially expressed genes; and determining the number of reads for each of the differentially expressed gene.

As embodied and broadly described herein, the present disclosure also relates to a method of identifying genetic element involved in microorganism adaptation to toxic organic compounds, comprising: generating reconstructed metabolic pathways using gene expression profiles, enriching the reconstructed metabolic pathway and assigning an enrichment score, and analyzing the internal transport and activation and initiation of degradation, through functional gene clustering of the gene expression profiles, to identify a genetic element that is activated upon exposure of the microorganism to toxic organic compounds.

As embodied and broadly described herein, the present disclosure also relates to a method of identifying genetic element involved in microorganism degradation of toxic organic compounds, comprising: overlaying gene expression profiles onto a pathway-genome database; and identifying pathways and enzymes related to degradation of toxic organic compounds.

As embodied and broadly described herein, the present disclosure also relates to a method of extracting organic compounds for selectively concentrating nitrogen-containing species from oil sands affected waters (OSPW), comprising: removing particulate matter from OSPW and acidifying the OSPW, liquid extracting the OSPW with an organic solvent one or more times, and evaporating the organic solvent and dissolving remaining organic matter in a solution containing a solvent and water.

As embodied and broadly described herein, the present disclosure also relates to a method of identifying genes, enzymes, or pathways involved in degradation activity of toxic organic compounds in a microorganism, comprising: generating gene expression profiles for the microorganism isolated from water affected with toxic organic compounds, identifying genes, enzymes, or pathways involved in adaptation of the microorganism to the compounds, and identifying genes, enzymes, or pathways involved in degradation of the compounds by the microorganism.

As embodied and broadly described herein, the present disclosure also relates to a method of RNA expression analysis for identifying genes, enzymes, or pathways involved in degradation activity of toxic organic compounds in a microorganism, comprising: calculating a score to determine upregulation of an enzyme associated with a pathway for degradation of toxic organic compounds based on RNA expression levels in response to the degradation of the toxic organic compounds, and identifying pathway enzymes, pathway inputs and terminal catabolites of the pathway.

As embodied and broadly described herein, the present disclosure also relates to a method for providing a gene expression profile being predictive for the specific response of a gene, enzyme or pathway comprising determining gene expression profiles from at least two microorganisms involved in the degradation activity of toxic organic compounds in the microorganisms.

As embodied and broadly described herein, the present disclosure also relates to a method of generating a gene expression profile of a microorganism involved with the biodegradation activity of toxic organic compounds comprising determining the expression levels, preferably RNA expression levels, of at least 2 genes, at least 5 genes, at least 10 genes, at least 15 genes, at least 20 genes, at least 25 genes, at least 30 genes, at least 50 genes, at least 100 genes, at least 200 genes, or at least 500 genes involved with the biodegradation of naphthenic acids fraction compounds (NAFC) or naphthenic acid (NA), and generating the gene expression profile based on said expression of the genes.

As embodied and broadly described herein, the present disclosure also relates to gene expression profile obtained by any of the methods as described herein.

As embodied and broadly described herein, the present disclosure relates to a polynucleotide comprising a nucleic acid having naphthenic acids fraction compounds (NAFC) degradation activity or naphthenic acid (NA) degradation activity, a vector expression system comprising such a polynucleotide and a host cell transformed with such a vector.

As embodied and broadly described herein, the present disclosure also relates to a genetic element comprising a gene, an enzyme or a pathway having naphthenic acids fraction compounds (NAFC) degradation activity or naphthenic acid (NA) degradation activity or a molecule that regulates a gene, an enzyme or a pathway having naphthenic acids fraction compounds (NAFC) degradation activity or naphthenic acid (NA) degradation activity.

As embodied and broadly described herein, the present disclosure also relates to an engineered microorganism comprising one or more of the polynucleotides as defined herein. As embodied and broadly described herein, the present disclosure also relates to a method for biodegradation of toxic organic compounds which are present in a contaminated environment, said method comprising: contacting the contaminated environment with a microbial consortium comprising at least one or more of the engineered microorganisms as defined herein; and maintaining the microbial consortium in contact with the contaminated environment for a time that is effective for the microbial consortium to biodegrade the toxic compounds.

As embodied and broadly described herein, the present disclosure also relates to a method of treating process-affected water to degrade toxic organic compounds, said method comprising: applying to the process-affected water containing the toxic organic compounds, an effective amount of one or more of the engineered microorganisms as defined herein, and monitoring removal of the toxic compounds from such application. In various embodiments, the method further comprises a photocatalyst.

As embodied and broadly described herein, the present invention also relates to a composition for biodegradation of toxic organic compounds, comprising: one or more of the engineered microorganism as defined herein, and an acceptable carrier for delivery of the engineered microorganism.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

### BRIEF DESCRIPTION OF THE DRAWINGS

While the specification concludes with claims particularly pointing out and distinctly claiming the disclosure, it is believed that the disclosure will be better understood from the following description of the accompanying figures wherein:

FIG. 1A is a graph showing relative abundance of different classes in NAFCs extracted from OSPW.

FIG. 1B is a graph showing NAs extracted from OSPW with general formula of C_{c}H_{2c+Z}O₂ and total concentration of 28.2 mg/L,

$$DBE = c - \frac{2 c + Z}{2} + 1 ,$$

and retention time in LC-Orbitrap

FIG. 1C is a chart showing microbial communities identified in OSPW based on their 16S rRNA and classified by their relative abundance.

FIG. 2A is a graph showing HPLC-Orbitrap results on relative abundance of different classes of NAFCs in OSPW before and after degradation by Pseudomonas cultures.

FIG. 2B is a graph showing degraded concentration (mg/L) of identified O2-NA compounds (i) was calculated as ΔCⱼ=Σ₁¹³¹(C₀ᵢ−C₁ᵢ) by isolated environmental strains j (1≤j≤5) on the phylogenetic tree; the numbers on the tree represented the branch length obtained from Phylogenetic analysis pipeline by ETE3.

FIG. 3A is a chart showing genes responsible for sensing, up-taking, and resisting NA compounds for environmental isolated strains from analysis of the Upper pathway in PS and PC (P. putida in co-culture).

FIG. 3B is a chart showing genes responsible for sensing, up-taking, and resisting NA compounds for environmental isolated strains from analysis of the Upper pathway in P. fluorescens Strain (FS), and FC (P. fluorescens in co-culture).

FIG. 3C shows some of the surrogates for the substrates identified in upper pathways differentially gene expression analysis and the corresponding genes.

FIGS. 4A-4D are graphs showing enrichment scores from a General pathways analysis on DEGs, revealing the physiological responses of Pseudomonas spp. to extracted NAs. All DEGs were enriched in KEGG pathways using DAVID Bioinformatics tools. Enriched pathways were classified based on the number of DEGs, p_value, and rich factor calculated by David Bioinformatics tool.

FIGS. 5A-5D show a Global carbon metabolism map comparison between P. fluorescens Strain (FS) and PC (P. putida in co-culture) degradation samples identifying the routes by which the carbon source (degraded NAs) can be pumped into central metabolism pathways.

FIGS. 6AA-6AH are charts showing a proposed model for biodegradation of NAFCs by Pseudomonas putida. indicating the group of NAFCs that the pathways and key enzymes have been investigated for: FIGS. 6AA-6AD-oxygen containing compounds; FIGS. 6AE-6AG-oxygen and nitrogen containing NAFCs; and FIG. 6AH-oxygen, nitrogen, and sulfur containing NAFCs.

FIGS. 6BA-6BI are charts showing a proposed model for biodegradation of NAFCs by Pseudomonas fluorescens, indicating the group of NAFCs that the pathways and key enzymes have been investigated for: FIGS. 6BA-6BE-oxygen containing compounds; FIGS. 6BF-6BH-oxygen and nitrogen containing NAFCs; and FIG. 6BI-oxygen, nitrogen, and sulfur containing NAFCs.

FIG. 7 is a graph showing LC50 96h toxicity results for extracted OSPW at 1× nominal concentration.

FIGS. 8A-8D are graphs showing analysis on the recalcitrancy and biodegradability of identified NA compounds based on ΔC of identified naphthenic acids compared in different retention times of LC columns (top), and DBE and carbon number of each compound (bottom); (A) Pseudomonas fluorescens, (B) Pseudomonas putida, (C) Pseudomonas stutzeri, (D) Pseudomonas sp., and (E) Rhodococcus sp.

FIG. 9 is a Venn diagram of differentially expressed genes in (A) P. putida strain (PS); (B) P. putida in co-culture (PC); (C) P. fluorescens Strain (FS), and (D) P. fluorescens in co-culture (FC).

FIGS. 10A-10D show Degradation pathways of alicyclic NAs degradation.

FIG. 11 is a diagram showing workflow of transcriptomics data analysis starting from DEGs in Pathway Tools to determine the transcriptional response of the Pseudomonas isolates in exposure to NAFCs.

### DETAILED DESCRIPTION

### a) Definitions

Unless otherwise defined, all technical and scientific terms used herein have the same meaning as commonly understood by a person of ordinary skill in the art to which the present invention pertains. As used herein, and unless stated otherwise or required otherwise by context, each of the following terms shall have the definition set forth below.

As used herein, terms of degree such as “about”, “approximately” and “substantially” mean a reasonable amount of deviation of the modified term such that the end result is not significantly changed. These terms may refer to a measurable value such as an amount, a temporal duration, and the like, and are meant to encompass variations of +/−0.1% of the given value, preferably +/−0.5%, preferably +/−1%, preferably +/−2%, preferably +/−5% or preferably +/−10%.

As used herein, articles such as “a” and “an” when used in a claim, are understood to mean one or more of what is claimed or described.

As used herein, the term “bacteria” refers to small prokaryotic organisms (linear dimensions of around 1 μm) with non-compartmentalized circular DNA and ribosomes of about 70 S. Bacteria protein synthesis differs from that of eukaryotes.

As used herein, the term “composition” includes genes, proteins, polynucleotide, peptides and pharmacological agents.

As used herein, the terms “comprises”, “comprising”, “include”, “includes”, “including”, “contain”, “contains” and “containing” are meant to be non-limiting, i.e., other steps and other sections which do not affect the end of result can be added. The above terms encompass the terms “consisting of” and “consisting essentially of”.

As used herein, the term “gene” refers to a nucleic acid molecule or a portion thereof, the sequence of which includes information required for the production of a particular protein or polypeptide chain. The polypeptide can be encoded by a full-length sequence or any portion of the coding sequence, so long as the functional activity of the protein is retained. A gene may comprise regions preceding and following the coding region as well as intervening sequences (introns) between individual coding segments (exons). A “heterologous” region of a nucleic acid construct (i.e., a heterologous gene) is an identifiable segment of DNA within a larger nucleic acid construct that is not found in association with the other genetic components of the construct in nature not present in the natural host.

As used herein, the term “genetic element” refers collectively to genes, enzymes, and pathways related to biological processes within an organism. The term may encompass one or more of these features in context, as appropriate.

As used herein, the term “genetic engineering” refers the use of genes, enzymes and pathways to guide modifications to the microorganism through processes such as for example rational engineering, directed evolution, or adaptive laboratory evolution.

As used herein, the terms “include”, “includes” and “including” are meant to be non-limiting.

As used herein, the term “polynucleotide(s)” refers to RNA, such as mRNA, or in the form of DNA, including, for instance, cDNA and genomic DNA obtained by cloning or produced by chemical synthesis or by a combination thereof. Preferably, the polynucleotides are recombinant polynucleotides. The DNA may be double-stranded or single-stranded. Single-stranded polynucleotides may be the coding strand, also known as the sense strand, or it may be the non-coding strand, also referred to as the anti-sense strand. Polynucleotides generally refers to any polyribonucleotide or polydeoxribonucleotide, which may be unmodified RNA or DNA or modified RNA or DNA. In addition, polynucleotide as used herein refers to triple-stranded regions comprising RNA or DNA or both RNA and DNA. The strands in such regions may be from the same molecule or from different molecules. The regions may include all of one or more of the molecules of a triple-helical region often is an oligonucleotide. Moreover, DNA or DNA comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those skilled in the art. The term “polynucleotide” as it is used herein embraces such chemically, enzymatically or metabolically modified forms of polynucleotides, as well as the chemical forms of DNA and RNA characteristics of viruses and cells.

As used herein, the words “preferred”, “preferably” and variants refer to embodiments of the disclosure that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

As used herein, the term “recombinant” means to recombined or new combinations of nucleic acid sequences, genes, or fragments thereof which are produced by recombinant DNA techniques and are distinct from a naturally occurring nucleic acid sequence.

As used herein, the term “transformation” refers to a process whereby exogenous or heterologous DNA (i.e., a nucleic acid construct) is introduced into a recipient host cell (e.g., prokaryotic cells). Therefore, in host cells, the acquisition of exogenous DNA into a host cell is referred to as transformation. With host cells, a stably transformed bacterial cell is one in which the introduced DNA has become integrated into a chromosome so that it is inherited by daughter cells through chromosome replication. This stability is demonstrated by ability of the host cell to establish cell lines or clones comprised of a population of daughter cells containing the introduced DNA.

As used herein, the expression “toxic organic compounds” refers to the group consisting of naphthenic acids fraction compounds (NAFC), naphthenic acid (NA), polycyclic aromatic hydrocarbons (PAH), benzene, toluene, ethyl benzene, xylenes, phenols, heavy metals, ions and a combination thereof, preferably naphthenic acids fraction compounds (NAFC) and naphthenic acid (NA).

As used herein, the term “vector” refers to a plasmid or phage DNA or other DNA sequence into which DNA can be inserted to be cloned. The vector can replicate autonomously in a host cell and can be further characterized by one or a small number of endonuclease recognition sites at which such DNA sequences can be cut in a determinable fashion and into which DNA can be inserted. The vector can be further contain a marker suitable for use in the identification of cells transformed with the vector. Markers, for example, are tetracycline resistance or ampicillin resistance. The words “cloning vehicle” are sometimes use for “vector”.

The present disclosure provides the methods and/or means to generate and analyze the gene expression profiles of microorganisms involved in the adaptation to and/or degradation of naphthenic acids fraction compounds (NAFC) or naphthenic acids (NA) typically found in contaminated environments such as, for example, Oil Sands Process-Affected water (OSPW) and ground water from oil sands development area.

The present disclosure also provides for the use of engineered microorganisms for reducing the amount of toxic organic compounds in a contaminated environment. For example, the present use may comprise exposing the contaminated environment containing the toxic organic compounds (e.g., naphthenic acids) to engineered microorganisms comprising an engineered nucleic acid encoding an enzyme having increased naphthenic acid degradation activity.

### b) Differential Gene Expression in Environmental Microbes

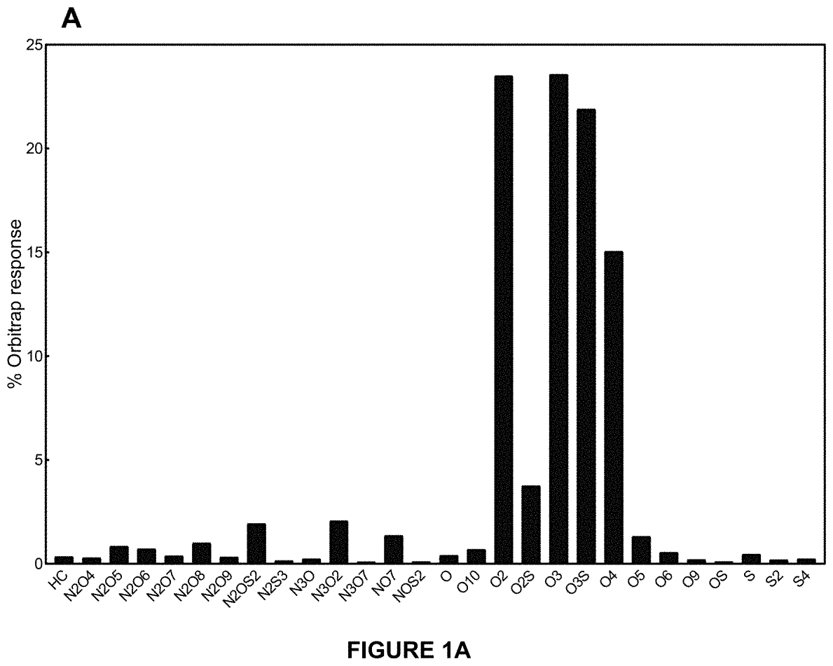

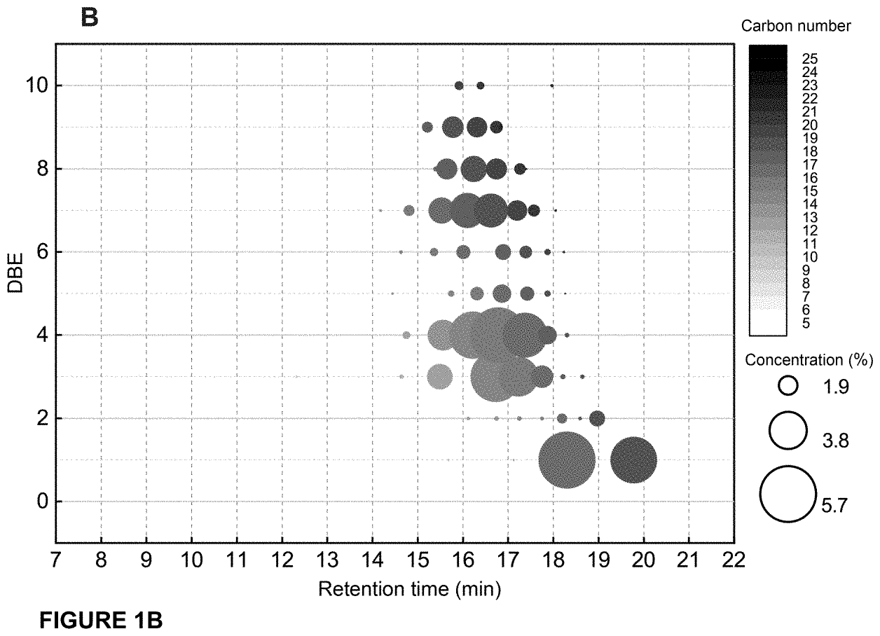

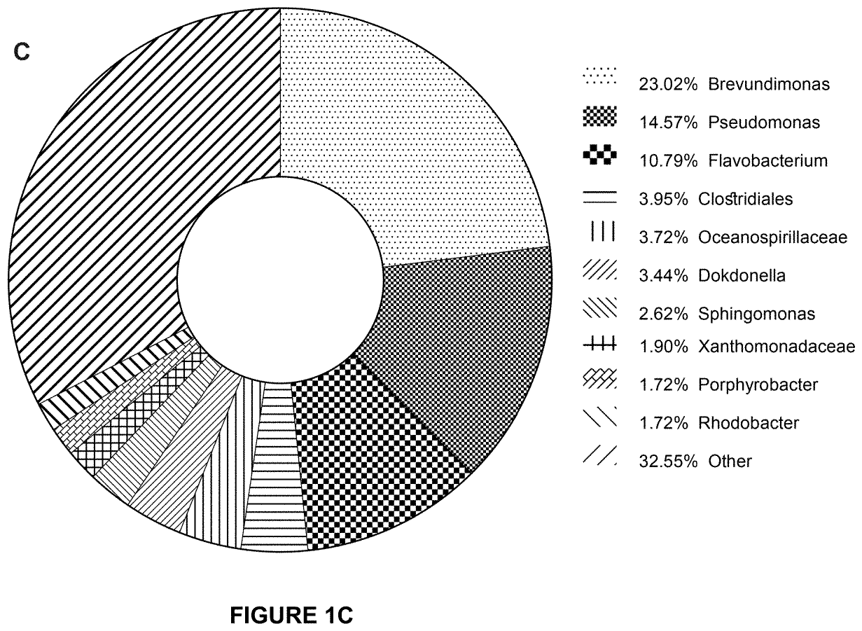

Environmental bacterial species are a promising means for removing toxic organic compounds from contaminated bodies of water. This is due, in part, to exposure to environmental contaminants, together with their tolerance for all sorts of physicochemical stresses (e. g. contact with oxidative stressors, temperature challenges, and sudden osmotic perturbations) and their ability to compete with predatory microbial species³². However, not all these microbial isolations are cultivable under standard laboratory conditions and therefore inaccessible for research. Further, recalcitrant NA compounds present a further challenge to effective degradation of toxic organic compounds. We hypothesized that microorganisms in OSPW might be able to degrade NAFCs but perhaps slowly and incompletely. FIG. 1C shows the microbial composition of OSPW. Availability of more biodegradable carbon sources compared to NAFCs could decrease the metabolic activities toward NAFCs biodegradation and favor the growth of non-degrading microbes. To ensure biodegradation of all classes of NAFCs, isolation and selection of effective microbes from OSPW is a key step. RNA-seq is a powerful tool to determine the transcriptomic response of bacteria upon exposure to NAFCs and provide insights into active degradation pathways and biodegradation mechanisms.

OSPW contains several major classes of contaminants including naphthenic acids (NAs), polycyclic aromatic hydrocarbons (PAHs), BTEX (benzene, toluene, ethyl benzene, and xylenes), phenols, heavy metals and ions¹⁰. Naphthenic acids fraction compounds (NAFCs) are the most toxic contaminants in oil sands process-affected water (OSPW). NAFCs are a mixture of several hundred chemical compounds with general formula CcHhNnOoSs, where c is carbon number (7≤c≤26), n, o and s represent the number of nitrogen, oxygen and sulfur atoms, respectively. Each NAFCs can be further categorized into Oo, NnOo, OoSs, NnSs, and NnOoSs based on the composition of heteroatoms in its empirical formula. Classic NAs (O2-NAs) have an empirical formula of CcHhO2 and are the most toxic class of NAFCs (FIGS. 1A and 1B). The water treatment technologies of OSPW are assessed based on degradation or removal of NAFCs, and NAs. Classic naphthenic acids (NAs), a subset of NAFCs, are the primary acute toxic constituent of OSPW^{2,4,11} and have long been defined as alkyl-substituted acyclic and cycloaliphatic carboxylic acids. By advanced mass spectrometry techniques^{12,13}, these chemical compounds can be broadly assigned to empirical formula C_{c}H_{2c+Z}O₂ where c is the number of carbons and Z is zero or an even negative integer representing the hydrogen deficiency due to rings or double bonds (double bound equivalent is:

$$DBE = c - \frac{2 c + Z}{2} + 1 {\text{)}}^{14 , 15} .$$

Further, HPLC-Orbitrap ultrahigh-resolution mass spectrometryl^{6,17} also confirm N- and S-containing potential heteroatomic compounds (C_{c}H_{2c+Z}NₙOₒSₛ)¹⁸.

The present inventors have identified and isolated 5 distinct bacterial strains from OSPW that showed the capability of assimilating extracted naphthenic acids through enrichment culture techniques. The five isolated strains, Pseudomonas fluorescens (P. fluorescens), Pseudomonas putida (P. putida), Pseudomonas stutzeri (P. stutzeri), Pseudomonas sp. (P. sp.), and Rhodococcus sp. (R. sp.), have been deposited at the International Depository Authority of Canada (IDAC), the P. fluorescens strain having the deposit number IDAC 170320-02 (P. flu #1-#10), the P. putida strain having the deposit number IDAC 170320-01 (P. putida #1 to #10), the P. stutzeri strain having the deposit number IDAC 170320-03 (P. stutzeri #1-#10), the P. sp. strain having the deposit number IDAC 170320-04 (P. sp #1-#10), and the R. sp. strain having the deposit number IDAC 170320-05 (Rhodo #1-#10). Changes in the chemosphere within OSPW changes were mapped during biodegradation within these strains using HPLC-Orbitrap mass spectrometry. Pseudomonas putida and Pseudomonas fluorescens were selected for further NAs biodegradation analysis. For the study, the transcriptomic response of the environmental Pseudomonas strains to extracted NAs using RNA-seq method was investigated. The study also established a novel differentially expression analysis on RNA-seq data that enabled the investigation of the mechanisms by which the strains sense, uptake, and resist NAs, physiological response of Pseudomonas strains to NA exposure, reveal NA biodegradation pathways, and key enzymes. Combination of genetic data with NAs analytical data enabled the elucidation of the NAs degradome, and provided insights into the new chemical structures that have not been detected previously by analytical methods.

The present disclosure, for the first time, provided a novel method of differential gene expression analysis of RNA-seq data as a response to NAs exposure. The analysis has identified novel transport system, and key genes responsive to NA compounds, biochemical pathways involved in NAs biodegradation, and generated new insights toward NA structures as a cognate substrate of expressed enzymes.

Inspection of the results revealed new genes, enzymes, and pathways that may serve as new targets for modification for increased degradation of toxic organic compounds and more effective remediation of contaminated waters, such as OSPW.

### c) Naphthenic Acids Fraction Compounds (NAFC) and Naphthenic Acid (NA) Characterization

The characterization of the naphthenic acid (NA) activity is assessed with the following method. Suspended particulate matter was removed using vacuum filtration through grade 4 glass fiber filters with a 1.2 μm nominal particle retention size (Fisher Scientific, Canada) from OSPW (in 1 L batches) provided by an industrial producer operating in the Athabasca oil sands, and stored in sealed polyethylene containers in the dark at 4° C. prior to testing. After filtration, the OSPW pH was lowered to pH 2 with concentrated H₂SO₄ and liquid-liquid extracted twice with 100 mL of dichloromethane (DCM). For analytical characterization of NAs, the extracted organic fraction was dissolved in 50:50 solution of Acetonitrile: Water at nominal concentrations of 1× the concentration of the original sample of OSPW. Analysis of extracted OSPW was performed using a HLPC-Orbitrap Elite mass spectrometer. Component separation was performed using an Ultimate 3000 HPLC system (Thermo Fisher Scientific, San Jose, Calif., USA) on a C8 column (150×3.0 mm, 3 μm particle size; Thermo Fisher Scientific, San Jose, Calif., USA) at 40° C. Flow rate is set at 0.5 mL/min and an injection volume of 5 μL is used.

The mobile phases consisting of (A) 0.1% acetic acid in water/methanol (90/10; v/v) and (B) 100% methanol will be employed. The following mobile phase composition is used: 5% B for 1 min, followed by a linear gradient ramp to 90% B at 10 min, to 99% over 5 min, and returning to 5% B in 1 min, followed by a 4 min hold prior to the next injection. The eluent is injected directly into the Orbitrap™ Elite. The Orbitrap™ is operated under source temperature of 350° C. in negative electrospray (ESI−) mode Sheath, auxiliary, and sweep gas flow at 30, 5 and 5 (arbitrary units), respectively. Capillary temperature and S-Lens RF are kept at 350° C., and 65%, respectively. Resolving power set to a nominal value of 120,000 at full width half-maximum at m/z 400 and using a full maximum ion time of 200 ms.

A commercial technical mixture of NAs (Refined Merichem) was used for calibration curves, and an authentic pure isotopically-labelled NA internal standard (Dodecanoic-D23 Acid) was added to each sample, blank, and standard prior to analysis. All chromatograms were processed automatically using Xcalibur™ 2.2 software/QuanBrowser (Thermo Fisher) using the same processing parameters such as integration type, smooth, peak-to-peak amplitude and peak detection for all samples. Manual integration was performed only when necessary. Total O2 naphthenic acid concentrations were calculated based on external calibration. A calibration curve was generated from serial dilutions of a stock commercial naphthenic acid solution (Refined Merichem). Total peak area ratios for all homologues, based on the sum peak area ratio (peak area of analyte divided by the peak area of the internal standard) for all identified homologues from C7 to C22 and DBE=1 to DBE=10 were fit to a least squares regression. Total naphthenic acid peak area ratios were then interpolated based on the linear calibration function, to provide concentrations for total naphthenic acids.

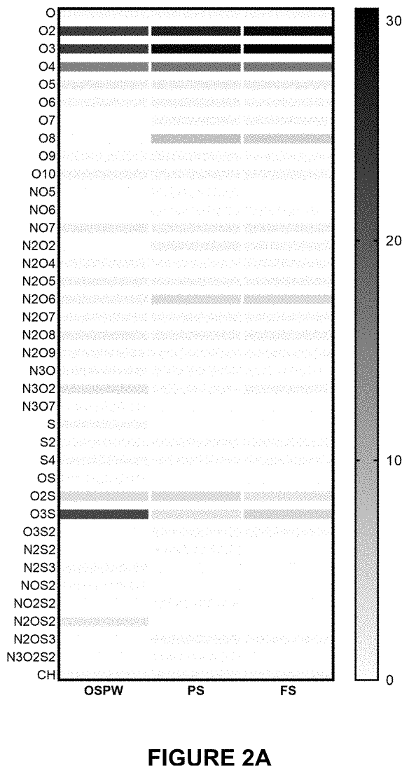

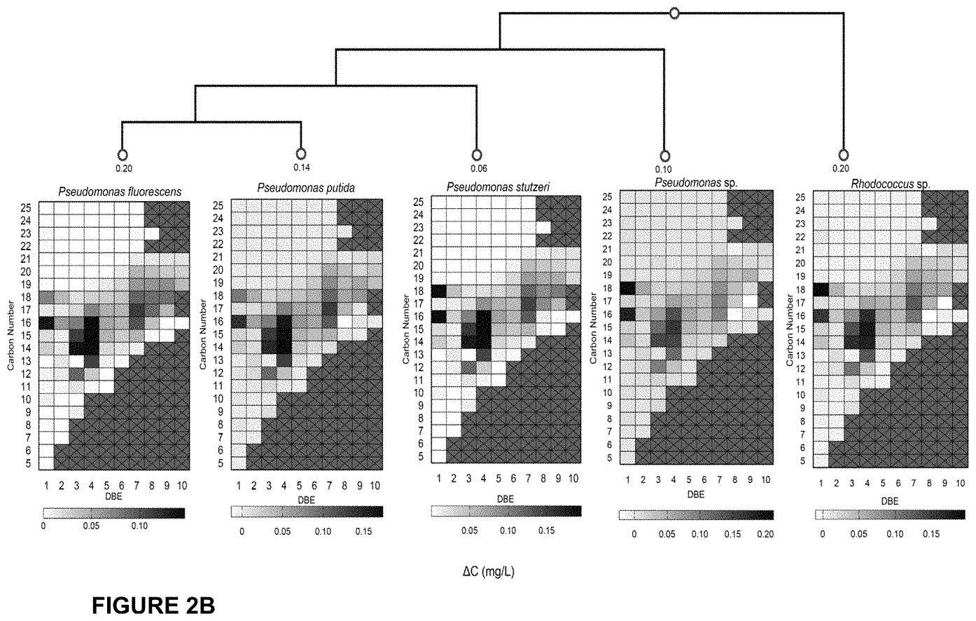

The HPLC-Orbitrap™ analysis results confirmed the Athabasca naphthenic acids signature and quantification was performed to investigate the relative abundances and concentration distribution of only O₂-NAs in mass spectrums. O₂-NAs comprise the majority of the NA species and correlate positively to the toxicity of OSPW. Measuring the concentration of NAFCs is a challenge due to lack of internal standard in HPLC-Orbitrap mass spectrometry. So, NAFCs are analyzed based on their abundance of peaks not exact quantification of their concentration (FIG. 2A).

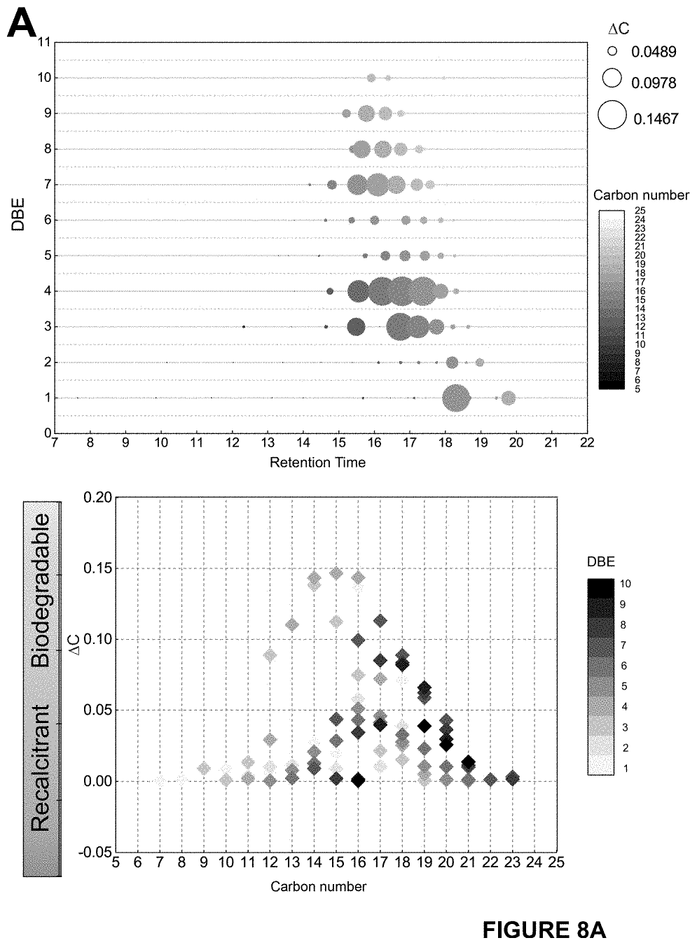

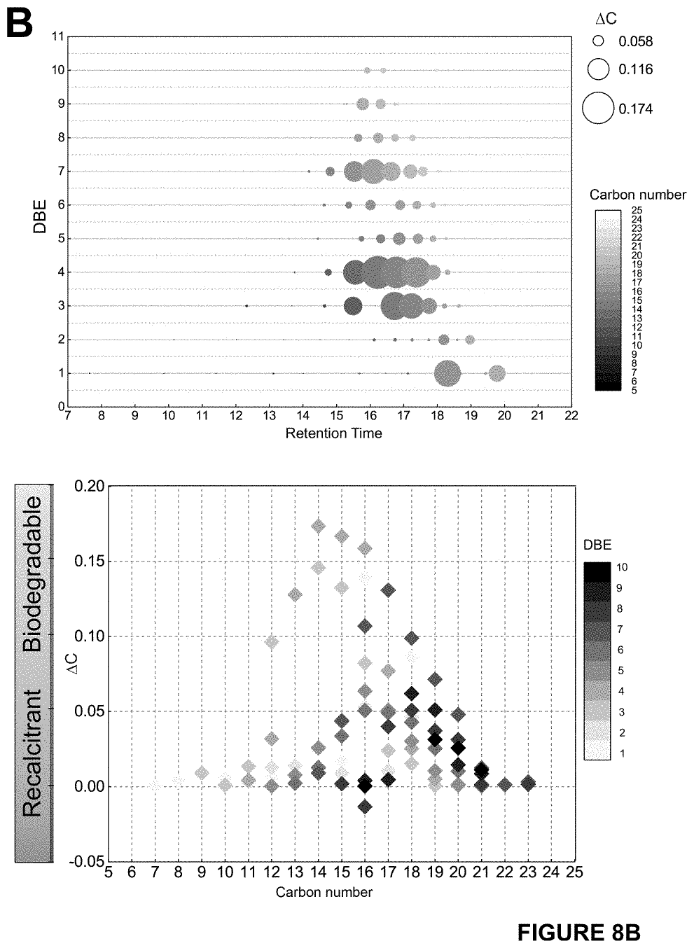

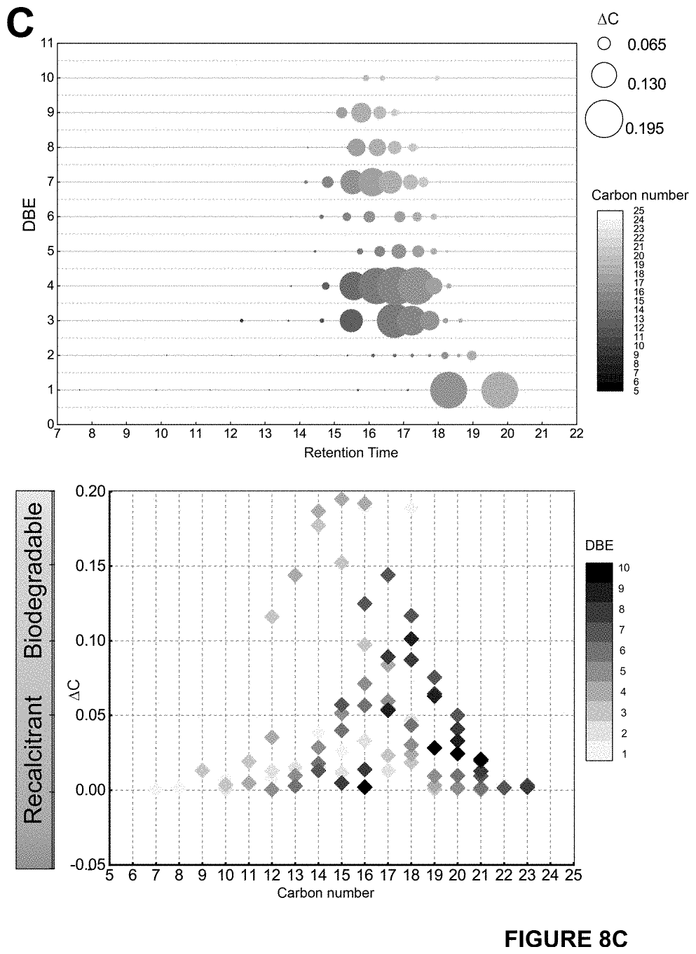

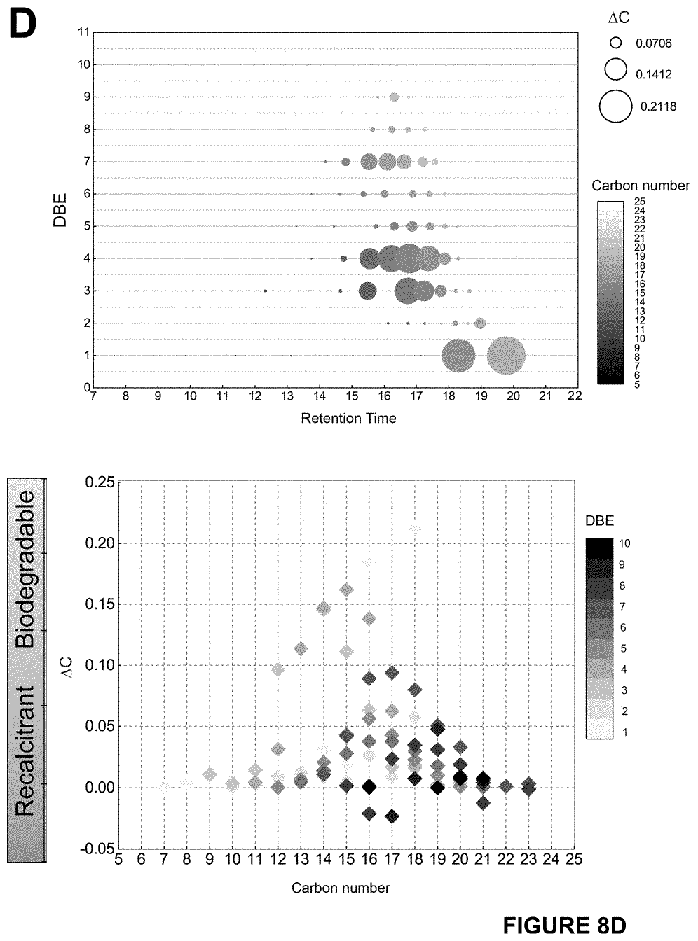

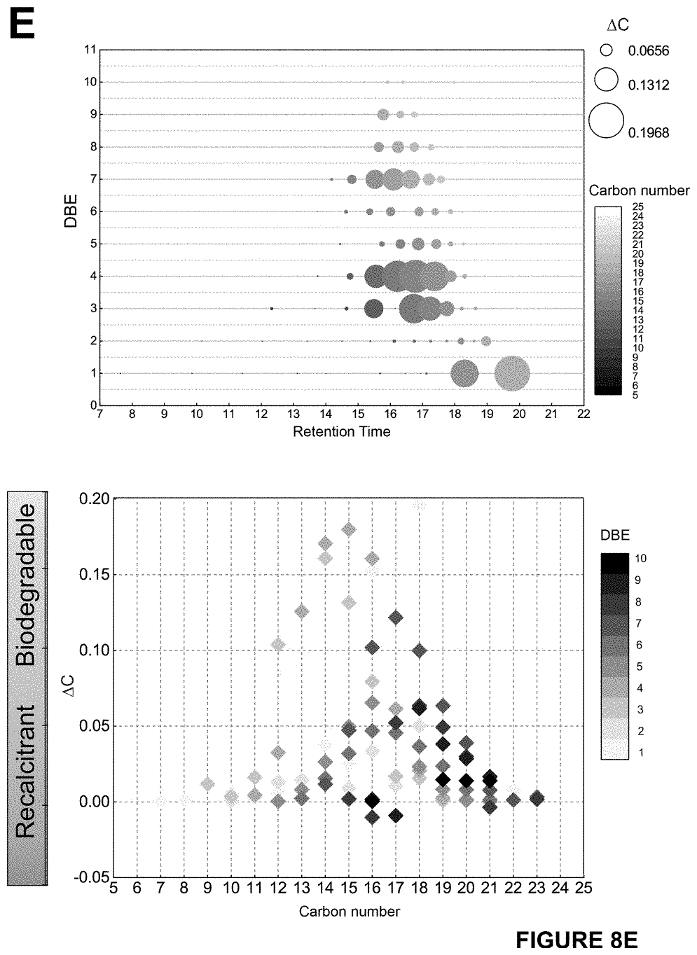

General formulae of CₙH_{2n+Z}O₂ have been assigned to 131 identified NA compounds with the total concentration of 28.2 mg/L ranging from 5≤n≤25 and 1≤DBE≤10 (FIG. 1B). Carbon number of 16 and DBE of 4 groups with 20.08 and 21.33% of total measured concentration showed the most abundant NA compounds. DBE<5 compounds include the aliphatic (DBE=1) and alicyclic (DBE=2, 3 and 4) compounds. Bicyclic and tricyclic NAs were found to be more difficult to degrade than aliphatic or monocyclic NAs using NA surrogates²⁰. DBE≥5 could be alicyclic or polycyclic aromatic compounds. Aromatic NAs are demonstrated to be more toxic to embryos of zebrafish compared to alicyclic NAs³³. Aliphatic and lower DBEs come in longer retention time while aromatic and polycyclic compounds eluted faster from the column. For example, C₁₆H₃₂O₂, and C₁₈H₃₆O₂ with retention times of 18.3 and 19.78, respectively, are potentially fatty acids with aliphatic structures. To have a more comprehensive insight toward recalcitrant NA compounds, we analyzed the degraded concentration (ΔC) of each compound with respect to DBE, carbon number, and retention time (FIG. 8A-8D). It can be observed that recalcitrancy is not only dependent on the chemical structure, but also the metabolic capacity of the strains. Pseudomonas putida and stutzeri, for example, have shown higher biodegradation capacity for DBE=8 (C16, C17, and C18). It can also be observed that NA compounds with C14, C15, and C16 at DBE=4 were biodegradable among all isolated strains. Except for C16, all NA compounds at DBE=2 showed low biodegradability that could be related to alicyclic NA compounds such as cyclohexane carboxylic acid (CHCA) and cyclohexane acetic acid (CHAA)³⁴.

### d) Microbial Community Characterization

All microorganisms in OSPW have been identified and quantified base on their relative abundance. The 16S rRNA gene V4 variable region PCR primers 515/806 were used in a single-step 30 cycle PCR using the HotStarTaq Plus Master Mix Kit (Qiagen, USA) under the following conditions: 94° C. for 3 minutes, followed by 30 cycles (5 cycle used on PCR products) of 94° C. for 30 seconds, 53° C. for 40 seconds and 72° C. for 1 minute, after which a final elongation step at 72° C. for 5 minutes was performed.

Sequencing was performed on an Ion Torrent PGM following the manufacturer's guidelines. Sequence data were processed using a proprietary analysis pipeline. In summary, sequences were depleted of barcodes and primers, then sequences <150 bp removed, sequences with ambiguous base calls and with homopolymer runs exceeding 6 bp were also removed. Noises were removed from sequences, operational taxonomic units (OTUs) were generated. OTUs were defined by clustering at 1% divergence (99% similarity). Final OTUs were taxonomically classified using BLASTn against a database derived from RDPII (http://rdp.cme.msu.edu) and NCBI (www.ncbi.nlm.nih.gov).

Analysis of the whole microbes in OSPW samples resulted in a collection of 176 bacteria which ten most abundant identities (67.43%) belonged to three different phyla and 9 families. The majority of identities were members of the Proteobacteria phylum (52.69%), followed by Bacteroidetes (10.79%), and Firmicutes (3.95%). Proteobacteria phylum is dominated by Caulobacterales (23.02%) and Gammaproteobacteria (14.57) classes which made the Brevundimonas and Pseudomonas identities, respectively (FIG. 1C). Brevundimonas sp. strain X08 were isolated from soils co-contaminated by cadmium (Cd) and polycyclic aromatic hydrocarbons (PAHs) in Northeast China³⁵. Some strains of Brevundimonas were also identified by growing on the naphthenic acids^{36,37}.

### e) NA-Degrading Strain Isolation and Biodegrading Experiment

To isolate microbial populations capable of assimilating NAs as a carbon source, a selection method is designed to trigger the active bacteria while hinder the growth of inactive microorganisms in exposure to NAs. Once strains were isolated, phylogenetic analysis was performed using ETE3 pipeline on KEGG database. For degradation samples, overnight grown cultures of the strains in M9 media supplied with glucose were added to fresh M9S media supplied with vacuum filtered non-extracted OSPW at the initial density of 0.1 OD₆₀₀ in 20 ml shaking flasks. After one-month incubation at 22° C., degradation samples were extracted and analyzed using LC-Orbitrap mass spectrometry as described above.

For strain isolation, 1 L of OSPW was filtered on 1.2 μm pore size, hydrophilic nylon membrane (Millipore Sigma) to remove the large particulates from water. Then, the collected water was filtered on 0.22 μm pore size, hydrophilic PVDF membranes (Millipore Sigma) to collect the microbial community. The collected biomass was scraped from filter and homogenized in sterilized M9 minimal salts 1× solution (11.28 g of Sigma Aldrich M9 salts in 1L sterilized water). M9 1× solution components were 6.8 g/L Na₂HPO₄, 3.0 g/L KH₂PO₄, 0.5 g/L NaCl, and 1 g/L NH₄Cl that supplemented with 0.1 mM CaCl₂), and 2 mM MgSO₄ to prepare the final media (herein designated M9 media). The homogenized biomass was inoculated in flask contained M9 media supplemented with 30 mg/L extracted NAs and 2 g/L D-glucose anhydrous (Sigma Aldrich).

This arrangement provides enriched culture with the mixture of growth supporting carbon source (glucose) and extracted NAs that are slowly degraded by native culture. This culture was transferred weekly to fresh medium (20% V_{inoculum}/V_{culture}) with increasing NAs supplement up to 150 mg/L and decreasing the glucose concentrating to zero during 5 weeks. The prepared culture then was serially diluted in M9 solution and plated on M9 agar plates (30% w/v agar:M9 1× solution) supplemented with 30 mg/L NAs. After 72 h incubation at room temperature, 22 single colonies were isolated from all the plates designated MT1-MT22. Colonies were grown in LB medium, and 22 glycerol stocks were prepared.

Genomic DNA was isolated from all colonies using Sigma Aldrich Bacterial Genomic DNA Kit based manufacturer's instructions, and full length 16S rRNA gene (1500 bp) was amplified to identify the colonies. The 27F and 1492R primers set were designed and used in PCR amplification using C1000 Touch Thermo Cycler (BIO-RAD). PCR cocktails for 50 μl reaction mixtures contained 1× reaction buffer (Standard Taq Reaction Buffer), 200 μM (10 mM dNTPs), 0.2 μM of each primer (10 μM forward and reverse primers), 1.25 U of DNA Polymerase (Taq), and 500 ng of genomic DNA extracted from MT1-MT22. PCR amplification was performed with a PTC-100 Thermal Cycler. After initial denaturation for 5 min at 95° C., PCR was performed with 30 cycles with 15 seconds at 95° C., 45 seconds annealing temperature 59° C., and 90 seconds at 68° C. This is followed by 5 minutes at 68° C. at final extension step.

After BLASTn of 16S rRNA sequences in NCBI, isolation of environmental strains on extracted NAs resulted in five unique strains which four belonged to Pseudomonas identity and one was identified as Rhodococcus strain (FIG. 2B). Four Pseudomonas strains were identified as fluorescens, putida, stutzeri, and uncharacterized (shown only as Pseudomonas sp.) strains. The entire 16S rRNA gene sequence for each of the five strains, Pseudomonas fluorescens (SEQ ID NO: 1), Pseudomonas putida (SEQ ID NO: 2), Pseudomonas stutzeri (SEQ ID NO: 3), Pseudomonas sp. (SEQ ID NO: 4), and Rhodococcus sp. (SEQ ID NO: 5), is provided. Interestingly, in the analysis of entire microbes in OSPW, Rhodococcus abundance was only 0.09% which was much less than Pseudomonas strain (14.57%). Degradation with all five strains was performed and analyzed using HPLC Orbitrap spectrometry. Concentration of each NAᵢ compound was measured before (C₀ᵢ) and after (C₁ᵢ) degradation. The degraded concentration of NAs (C₀ᵢ-C₁ᵢ) was calculated and plotted as heat maps for each strain. Total degraded NA concentration for each strain (1≤j≤5) was calculated as ΔCⱼ=Σ₁¹³¹(C₀ᵢ−C₁ᵢ). Among the identified strains, Pseudomonas stutzeri showed the highest degraded concentration with ΔC=3.9 mg/L followed Rhodococcus with 3.2 mg/L. While Pseudomonas putida and fluorescens showed identical total degrade NA concentration 3.1 mg/L, the uncharacterized Pseudomonas strain showed the lowest ΔC=2.7 mg/L.

C16 and C18 at DBE=1, which were previously discussed as potential Palmitic (C₁₆H₃₂O₂) and Stearic acids (C₁₈H₃₆O₂), have high shown degradation (dark) in all samples. This confirms the aliphatic structure led to higher biodegradability of these compounds. The biodegradation probability of palmitic and stearic acids has been estimated using EBI Suite Biowin2 non-linear model as 0.86 and 0.81, respectively, with probability greater than zero is interpreted as ‘likely to biodegrade rapidly’⁴¹.

The concentration of C₁₆H₁₈O₂ (DBE=8) increased in P. putida, Pseudomonas sp. and Rhodococcus sp. (white heat maps in FIG. 2B). A similar trend was observed for C₁₇H₁₈O₂ (DBE=9) and C₂₁H₂₈O₂ (DBE=8) in both Pseudomonas sp. and Rhodococcus strains. P. fluorescens and P. stutzeri have shown identical degradation patterns in most of the carbon numbers and DBEs. While Pseudomonas sp. and Rhodococcus sp. have also similarly degraded naphthenic acid compounds, P. putida showed unique degradation pattern.

### f) Bioinformatics and Genome Assembly

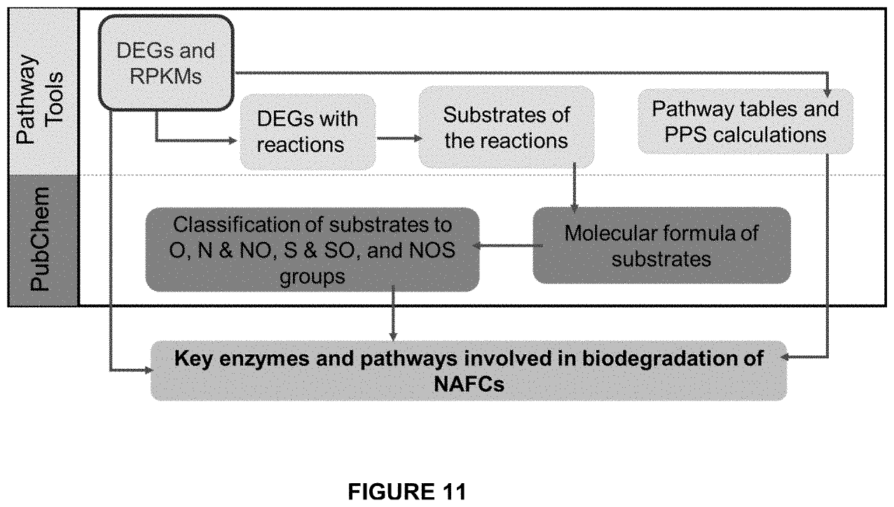

From the isolated environmental strains in the last section, P. putida and P. fluorescens were selected for RNA extraction due to higher growth rates in laboratory conditions and higher percentages of degraded NAs. We developed a systematic approach to investigate and classify the key enzymes and pathways for all fractions of NAFCs including Oo, NnOo, OoSs, NnSs, and NnOoSs based on the heteroatoms composition of the substrates of expressed enzymes (FIG. 11). Three tester samples of M9S media supplied with extracted NAs (10× nominal concentration) were inoculated with P. putida strain (PS), P. fluorescens Strain (FS), and a consortium comprising an equal number of cells of both strains (PC-FC) at an initial cell density of 0.1 (OD₆₀₀). The driver samples were identical to tester samples with the exception of NAs supplement that was replaced by additional M9S media. Total RNA was isolated from all six testers and drivers using ThermoFisher PureLink RNA Mini Kit according to the product instructions. Poly (A) polymerase (NEB) was used to enrich mRNA in RNA pool.

Suppression Subtractive Hybridization (SSH) analysis was employed to prepare library of differentially expressed genes (DEGs) upregulated in tester samples as compared to the corresponding driver. The major advantage of SSH is the combination of normalization and subtraction, which can both identify abundant differentially expressed genes and enrich rare transcripts to facilitate the identification of novel genes. Moreover, this technique eliminates the limitations of global gene expression analysis as it does not require complete genomic sequence information. A total of 2 μg RNA per sample was used as the input material to synthesize cDNA, amplify DEGs, and generate three RNA-seq libraries using Clontech® PCR-Select™ cDNA Subtraction Kit, USA. Sequencing libraries were constructed using next generation RNA-Seq platform (Genewiz, USA). These libraries and the corresponding differentially expressed gens for P. putida, and P. fluorescens strains, as well as their consortia were called PS, FS, and PC, and FC, respectively.

Raw reads were first processed through Trimmomatic to remove adapter sequences, sequence ends with poor base-qualities, and entire reads which were of poor quality⁷³. Digital normalization using the Khmer software was performed to remove sequencing reads redundant beyond 255-times coverage as well as reads containing low-abundance k-mers⁷⁴ (minimum abundance threshold was 20). Transcriptome assembly was accomplished using rnaSPAdes (de novo Genome Assembly method)⁷⁵ with k=55, no mismatch correction and no coverage cut-off as these were normalized data. Prior to ORF predictions, the assembled genome database was processed using MetaPathways pipeline to renames input contigs with standardized identifiers and removes and any contigs smaller than 180 base pairs. Subsequently, MetaPathways was used to predict the open reading frames (ORFs). Predicted ORFs less than 180 base pairs or 60 amino acids in length are removed by default from downstream analysis to achieve more reliable alignment and annotation outcomes. MetaPathways⁷⁶ ensures all annotations meet certain quality thresholds, and has default annotation parameters for FAST/LAST and BLAST that are fairly conservative (Length: 180 bp (60 aa), E-value: 1e-6, bit-score: 20, Bit-score ratio >0.4). Three differentially genes expression browsers of P. putida (PS) and P. fluorescens (FS) single strains as well as Pseudomonae consortia (PC-FC) have been used from MetaCyc annotations for further analysis of pathways. Pseudomonas putida KT2440, and Pseudomonas fluorescens F113 were used as reference genome and reads per kilobase of transcript per million (RPKM), reflecting both the effect of sequencing depth and gene length for the read count, values were calculated.

The transcript changes in P. putida, P. fluorescens, and their consortia with and without exposure to NAs were investigated using RNA-seq analysis. For each culture, the subtractive cDNA library was constructed and sequenced with 13000 times coverage for each library. After digital normalization of original reads, three minimum abundance thresholds were used for each sample: 10, 20 and 100. Each of these nine datasets were assembled and the number of transcripts assembled was negatively correlated with the minimum abundance threshold. Only assemblies built with a minimum abundance threshold of 20 were chosen for downstream analysis in MetaPathways. After ORF prediction and QC, 619, 692, 901 translated ORFs were predicted for P. putida, P. fluorescens, and consortia, respectively. Furthermore, ORFs have been annotated by aligning FAST, a multi-threaded version of LAST, to databases including COG, KEGG-Uniport, MetaCyc, and Ref-Seq. For each database, the best hit (if a hit exceeding alignment quality thresholds exists) is given as a database specific ORF annotation (Tables 1A and B). Annotations from MetaCyc database were used for further analysis of RNA-seq results.

TABLE 1A
Open Reading Frame (ORF) Prediction Statistics

| | PS | FS | PC-FC |
|---|---|---|---|
| Number of sequences in input file before | 1003 | 1125 | 1674 |
| QC (nucleotide) | | | |
| minimum length | 87 | 89 | 14 |
| average length | 427 | 422 | 387 |
| maximum length | 5822 | 5963 | 4995 |
| total base pairs | 428778 | 475321 | 638641 |
| Number of sequences after QC (nucleotide) | 620 | 650 | 901 |
| minimum length | 180 | 180 | 180 |
| average length | 611 | 629 | 599 |
| maximum length | 5822 | 5963 | 4995 |
| total base pairs | 379275 | 409082 | 539780 |
| Number of translated ORFs before | 827 | 911 | 1194 |
| QC (amino acids) | | | |
| minimum length | 20 | 19 | 19 |
| average length | 117 | 115 | 117 |
| maximum length | 1038 | 1126 | 990 |
| total base pairs | 97559 | 105044 | 140001 |
| Number of translated ORFs after | 619 | 692 | 901 |
| QC (amino acids) | | | |
| minimum length | 60 | 60 | 60 |
| average length | 143 | 138 | 141 |
| maximum length | 1038 | 1126 | 990 |
| total base pairs | 88712 | 95852 | 127682 |

TABLE 1B
Annotations Statistics

| | PS | FS | PC-FC |
|---|---|---|---|
| Total protein annotations COG | 1218 | 1517 | 1986 |
| Number of ORFs with hits in COG | 289 | 362 | 466 |
| Total protein annotations kegg-uniport | 1117 | 1469 | 1840 |
| Number of ORFSs with hots in kegg-uniport | 245 | 311 | 399 |
| Total protein annotations metacyc | 266 | 337 | 364 |
| Number of ORFs with hits in metacyc | 82 | 122 | 143 |
| Total protein annotations refseq | 2238 | 2689 | 3450 |
| Number of ORFs with hits in refseq | 480 | 572 | 728 |
| Total protein annotations eggnog | 1979 | 2321 | 2958 |
| Number of ORFs with hits in eggnog | 442 | 511 | 651 |
| protein annotations from COG | 289 | 362 | 466 |
| protein annotations from kegg-uniport | 245 | 311 | 399 |
| protein annotations from metacyc | 82 | 122 | 143 |
| protein annotations from refseq | 480 | 572 | 728 |
| protein annotations from eggnog | 442 | 511 | 651 |
| Total protein annotations | 514 | 588 | 748 |

### g) Differential Gene Expression (DEG) Analysis

Because of the poorly characterized pathways of NA degradation, and the aim of finding promising degrading genes for a degrading recombinant microorganism, at this stage, all DEGs were considered as significantly expressed. Analysis of DEGs is broken down into four categories: upper pathways, general pathway, degradation pathway, identification of key enzymes, and pathway predictions for their cognate substrates.

The identity of genes were converted to Entrez Gene ID using Pathway Tools⁶⁹ Smart Tables in Pseudomonas putida KT2440, and Pseudomonas fluorescens F113 Pathway Genome Databases (PGDB). For upper pathways analysis, four lists of DEGs were functionally clustered using DAVID Bioinformatics⁷⁷ tool to identify the DEGs from outer membrane, ATP-binding cassette (ABC) transporters, major facilitator superfamily (MFS) transporters, efflux pumps, and active oxygen species (AOS) clusters. Physiological response of Pseudomonas spp. to NA exposure was analyzed by reconstructing the general pathways using KEGG mapper tool (www.genome.jp/kegg/tool/map_pathway.html) with DEGs assigned to KO numbers by means of Uniport Retrieve/ID mapping tool (www.uniprot.org).

Reconstructed pathways were enriched using DAVID Bioinformatics tool. Threshold of gene counts and the Expression Analysis Systematic Explorer (EASE) score were set at 5 and 0.4, respectively. The degradation pathways analysis, however, was impossible using KEGG due to inclusive pathway maps in KEGG, incomplete KO assignment, and having no organism-specific maps. On this basis, Pathway Tools software from MetaCyc were used for further degradation pathways analysis. Table of Pathways Diagrams were reconstructed by overlaying PS and PC as well as FS and FC RNA-seq data on the corresponding PGDBs using Omic Viewer tool in Pathway Tools, and degradation pathways related to NA compounds were identified. In this procedure, some of the DEGs have been automatically removed from the list due to uncharacterized pathway or unknown function of genes.

Knowing that the key enzymes in degradation pathways are responsible for oxidoreductase activities, PS and PC as well as FS and FC gene lists were combined, and functional clustering the genes was performed using DAVID Bioinformatics to identify the DEGs from oxidoreductase activity and domain clusters. This additional step of analysis enables comprehensive characterization of all the candidate key enzymes without knowing the pathways. To further analyze the pathways by which the key enzymes participate in NA degradations, the key enzymes with specific substrates that could be assigned to NA structures were identified. These substrates were used to predict the pathways using EAWAG-BBD/PPS, Swiss Federal Institute of Aquatic Science and Technology combined with University of Minnesota Biocatalysis/Biodegradation Database and Pathway Prediction System (http://eawag-bbd.ethz.ch) with the first reaction catalyzed by the corresponding key enzyme.

Comprehensive analysis on DEGs and conclusion on degradation pathways for complex environmental xenobiotic mixture such as NA compounds can be challenging due to unknown structures of different compounds, and lack of an accurate as well as quantifiable commercial mixture representative of Athabasca NA mixture. This analysis can be more complicated when it comes to enzymes with unknown functions, hypothetical gens, and poorly characterized pathways in Pseudomonas Pathway Genome Databases (PGDBs).

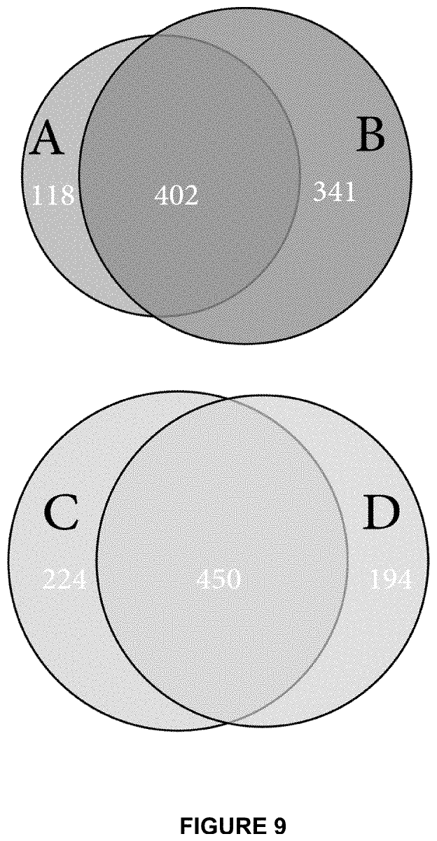

The present disclosure, for the first time, provided a novel method of differential gene expression analysis of RNA-seq data as a response to NAs exposure. The analysis has identified novel transport system, and key genes responsive to NA compounds, biochemical pathways involved in NAs biodegradation, and generated new insights toward NA structures as a cognate substrate of expressed enzymes. The RNA-seq results yielded four lists of 674, 644, 520, and 743 DEGs for FS, FC, PS, and PC, respectively, in response to NA compounds exposure (FIG. 9). Of these, 194, and 341 new genes have been newly expressed in FC and PC, respectively as compared to FS and PS. All these genes were upregulated in tester samples when exposed to extracted NAs. The average RPKM for PS, FS, PC, and FC were 1320.97, 1239.22, 532.44, and 304.82, respectively.

### h) Upper Pathways Analysis

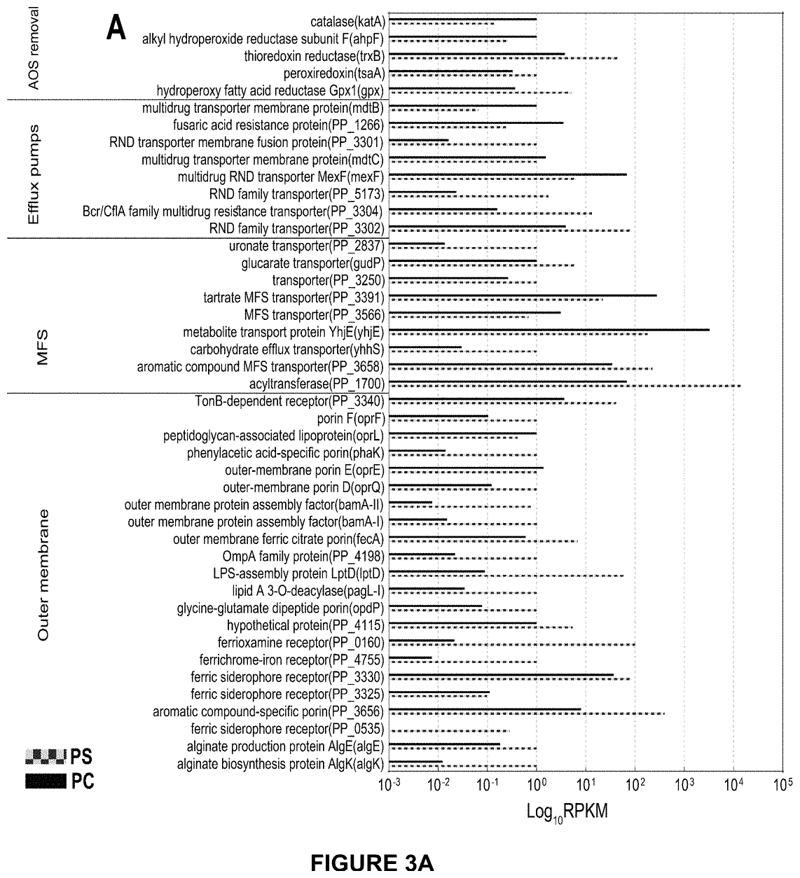

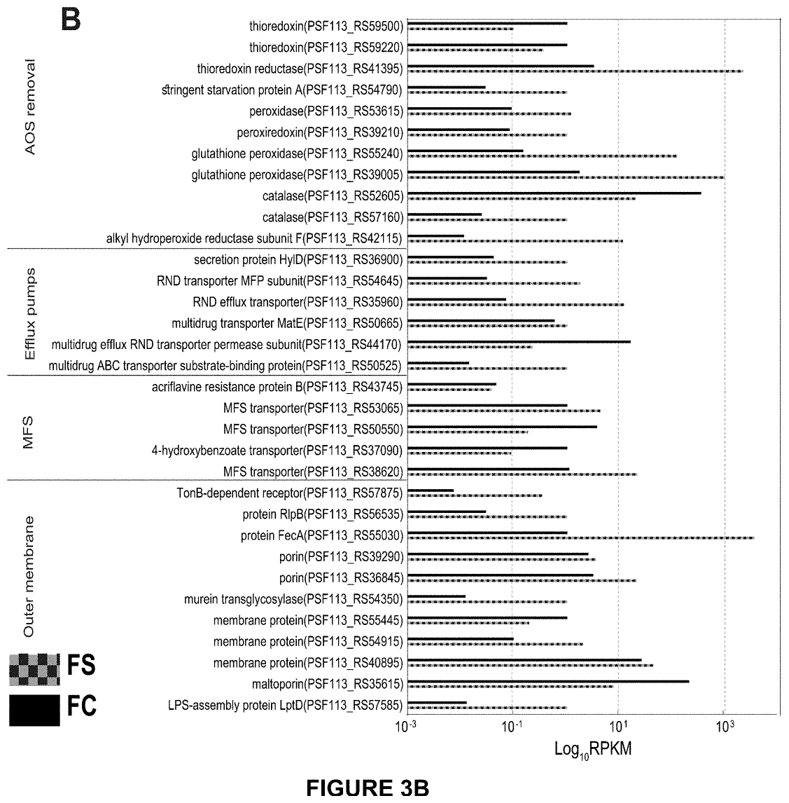

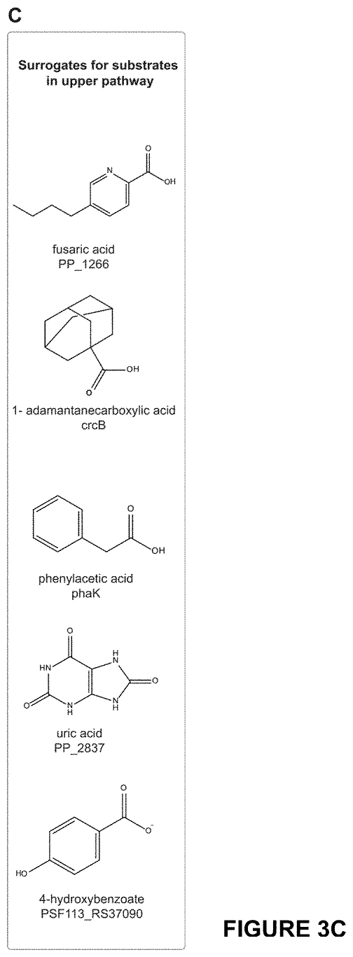

The capability of the cells to sense, uptake, and resist NA compounds and eventually activate the biodegradation pathways is of great importance in growth-linked biodegradation. RNA-seq analysis was started from the transport of NA compounds across the cell membrane to track the NAs before entering the degradation pathway. While all the extracted clusters are shown in FIGS. 3A and 3B, only ABC transporter genes are shown in Tables 2A-2D due to a large number of genes in this cluster.

TABLE 2A
ABC Transporter gene clusters extracted from P. putida strain (PS)
ABC Transporters

ABC transporter ATP-binding protein(urtE)
ABC transporter permease(PP_3077)
ABC transporter permease(PP_4149)
ABC transporter permease/ATP-binding protein(PP_2240)
ABC transporter substrate-binding protein(PP_3078)
amino acid ABC transporter permease(yhdY)
amino acid ABC transporter-binding protein YhdW(yhdW)
branched-chain amino acid ABC transporter permease(livM)
cation ABC transporter permease(PP_3803)
choline/betaine/carnitine ABC transporter ATP binding protein(cbcV)
choline/betaine/carnitine ABC transporter permease(cbcW)
dipeptide ABC transporter substrate-binding protein(dppA-III)
lipid A export ATP-binding/permease MsbA(msbA)
methionine ABC transporter substrate-binding protein(metQ)
phosphate ABC transporter permease(PP_5328)
spermidine/putrescine ABC transporter permease(spuG)
spermidine/putrescine ABC transporter substrate-binding protein(spuD)
sulfate ABC transporter permease CysT(cysU)
sulfate ABC transporter substrate-binding protein(sbp-II)
sulfate/thiosulfate ABC transporter ATP-binding protein(cysA)

TABLE 2B
ABC Transporter gene clusters extracted from P. putida in co-culture (PC)
ABC Transporters

ABC transporter ATP-binding protein(urtE)
ABC transporter ATP-binding protein(yehX)
ABC transporter permease(PP_4148)
ABC transporter permease(PP_4149)
BraC-like branched-chain amino acid ABC transporter substrate-binding
protein(PP_4867)
D-ribose ABC transporter permease(rbsC)
amino acid ABC transporter permease(yhdY)
amino acid ABC transporter-binding protein YhdW(yhdW)
cation ABC transporter permease(PP_3803)
choline/betaine/carnitine ABC transporter ATP binding protein(cbcV)
glutamate/aspartate ABC transporter permease(gltJ)
glutamate/aspartate ABC transporter substrate-binding protein(gltI)
iron ABC transporter substrate-binding protein(PP_4881)
lipid A export ATP-binding/permease MsbA(msbA)
methionine ABC transporter substrate-binding protein(metQ)
peptide ABC transporter substrate-binding protein(PP_4146)
phosphate ABC transporter permease(PP_5328)
phosphate-binding protein(PP_3818)
putrescine-binding protein(potF-I)
putrescine-binding protein(potF-IV)
ribose ABC transporter-ATP-binding subunit(rbsA-I)
spermidine/putrescine ABC transporter permease(spuG)
spermidine/putrescine ABC transporter substrate-binding protein(spuD)
sugar ABC transporter substrate-binding protein(PP_2264)
sulfate ABC transporter substrate-binding protein(sbp-I)
sulfate ABC transporter substrate-binding protein(sbp-II)
toluene tolerance protein(ttg2D)

TABLE 2C
ABC Transporter gene clusters extracted from P. fluorescens strain (FS)
ABC Transporters

ABC transporter ATP-binding protein(PSF113_RS34915)
ABC transporter substrate-binding protein(PSF113_RS46315)
ABC transporter substrate-binding protein(PSF113_RS58935)
ABC transporter(PSF113_RS37980)
ATP-binding protein(PSF113_RS32935)
LPS export ABC transporter permease LptG(PSF113_RS54385)
amino acid ABC transporter ATP-binding protein(livG)
amino acid ABC transporter ATPase(PSF113_RS36680)
amino acid ABC transporter permease(PSF113_RS54475)
glycine/betaine ABC transporter ATP-binding
protein(PSF113_RS34820)
histidine/lysine/arginine/ornithine ABC transporter permease
HisM(PSF113_RS36520)
histidine/lysine/arginine/ornithine ABC transporter permease
HisQ(PSF113_RS36525)
microcin C ABC transporter permease YejB(PSF113_RS46975)
microcin C ABC transporter permease YejB(PSF113_RS48785)
peptide ABC transporter permease(PSF113_RS34865)
peptide ABC transporter(PSF113_RS34860)
putrescine/spermidine ABC transporter ATP-binding
protein(PSF113_RS58930)
spermidine/putrescine ABC transporter substrate-binding
protein(PSF113_RS58940)
sulfonate ABC transporter(ssuC)
taurine ABC transporter substrate-binding protein(tauA)

TABLE 2D
ABC Transporter gene clusters extracted from P. fluorescens in co-culture
(FC)
ABC Transporters

ABC transporter permease(PSF113_RS40520)
ABC transporter substrate-binding protein(PSF113_RS53250)
ABC transporter substrate-binding protein(PSF113_RS60020)
ABC transporter(PSF113_RS37980)
ATP-binding protein(PSF113_RS32935)
LPS export ABC transporter permease LptG(PSF113_RS54385)
Lacl family transcriptional regulator(PSF113_RS40510)
MacB family efflux pump subunit(PSF113_RS39730)
glycine/betaine ABC transporter ATP-binding
protein(PSF113_RS34820)
glycine/betaine ABC transporter substrate-binding
protein(PSF113_RS51985)
glycine/betaine ABC transporter substrate-binding
protein(PSF113_RS58090)
histidine ABC transporter ATP-binding protein(PSF113_RS53235)
microcin C ABC transporter permease YejB(PSF113_RS46975)
microcin C ABC transporter permease YejB(PSF113_RS48785)
multidrug ABC transporter substrate-binding
protein(PSF113_RS50525)
putrescine/spermidine ABC transporter ATP-binding
protein(PSF113_RS58930)
ribose ABC transporter ATPase(PSF113_RS40515)
spermidine/putrescine ABC transporter substrate-binding
protein(PSF113_RS58940)
sugar ABC transporter ATP-binding protein(PSF113_RS43090)
sugar ABC transporter permease(PSF113_RS40865)
sugar ABC transporter permease(PSF113_RS43095)
sugar ABC transporter permease(PSF113_RS44100)
sulfonate ABC transporter(ssuC)
urea ABC transporter ATP-binding protein(PSF113_RS40735)
zinc transporter(PSF113_RS30385)

Transport of NAs across the outer membrane is accomplished via specific outer membrane protein channels and porins which typically transport the substrates based on the size and charge of the molecules (FIGS. 3A and 3B). In this cluster, TonB-dependent receptors, which can take up macromolecular complexes such iron-siderophore complexes, other metal-chelator complexes (Mb, Co, Cu), vitamin B₁₂ and sulfate esters, showed expression in all degradation samples. Expression of this membrane protein in both Pseudomonas spp. may be associated to transport of recently identified SO₄ compounds in OSPW², Metalloporphyrins found in oil feedstock⁴², or iron-limiting conditions due to expression of fecA in this category⁴³. Exposure of Pseudomonas putida to NA compound mixture resulted in expression of several porins including phaK (phenylacetate uptake) (FIG. 3B), oprF (non-specific large substrates), oprE (anaerobically induced porin required for growth on phenyl acetic acid-like molecules), oprQ (unknown substrates), oprL (Peptidoglycan-associated lipoprotein), opdP (Glycine-glutamate dipeptide porin), and PP_3656 (specific aromatic porin). P. fluorescens also showed transport across the outer membrane with the aid of porins including oprF (PFS113_RS39290), oprB (PSF113_RS36845), and maltoporin (PSF113_RS35615). OprB is positively regulated by its substrates glucose, fructose, glycerol, and mannitol while maltoporin is indispensable for the uptake of maltodextrins.

NA compounds can be transported across the cytoplasmic membrane by the ATP-binding cassette (ABC) superfamily (supporting information 1) and the major facilitator superfamily (MFS) (FIGS. 3A and 3B), also called the uniporter-symporter-antiporter family⁴⁴. Several MFS transporters for which expression can be associated with NA compounds were identified. For example, PP_3658 is benzoate transport protein from Aromatic Acid: H⁺ symporter (AAHS family); PP_3391 is tartrate MFS transporter from Anion:Cation Symporter (ACS family) in which the identical gene NP_001096662 has been differentially expressed⁴⁵ in male fathead minnow when exposed to OSPW; yhjE has unknown function from Metabolite H⁺ symporter (MHS) family and in the phylogenetic tree is far from other compounds of this family; gudP is glucarate transporter from ACS family which can either transport organic or inorganic anions. Among the organic anions transported are glucarate, hexuronates, phthalate, allantoate, and probably tartrate; PP_2837 is uronate (FIG. 3B) transporter probably from ACS family⁴⁴; PP_3250 is ATP:ADP antiporter (AAA family); PP_4001 is pore ion channel fluoride ion transporter CrcB. Over expression of this gene is related to protection of the chromosome from condensation by camphor⁴⁶ which resembles the diamond structure of NA compounds including Adamantanecarboxylic acid⁴⁷ (FIG. 3B) with methyl branches; PSF113_RS53065 has unknown function; PSF113_RS37090 is from AAHS family for aromatic acid transport (FIG. 3B).

Due to toxicity of some NA compounds to Pseudomonas cells, there are several levels of responses to toxic intracellular compounds that include structural alterations, general stress and metabolic responses, and active extrusion mechanisms⁴⁸.

Efflux pumps (FIGS. 3A and 3B) are considered the most efficient mechanism of toxic compounds tolerance in Gram-negative bacteria. This mechanism consists of pumping excess toxic compound present in the cell (membrane, periplasm or cytoplasm) to the outer medium, effectively preventing chemicals from reaching lethal concentrations in the cell. Among the different families of efflux pumps, secondary multidrug transporters with the subdivisions of the MFS, small multidrug resistance (SMR) family, the resistance-nodulation-cell division (RND) family, and the multidrug and toxic compound extrusion (MATE) family⁴⁹, is of great importance in xenobiotic transport. For example, yhhS is carbohydrate efflux transporter; PP_0961 is ABC transporter as efflux pumps that is involved in the inducible resistance to toluene and is present only in strains which carry the tod pathway for toluene degradation⁴⁸. MexF (MexEF pump) is involved in butanol and formaldehyde efflux; PP_1266 is fusaric acid (FA) (FIG. 3B) resistance protein⁵⁰.

Highly FA resistant strains were found only in Gram-negative bacteria, mainly in the genus of Pseudomonas; mdtB and mdtC⁵¹ are multidrug efflux system which confer resistance against novobiocin and deoxycholate; Bcr/CflA is drug resistance proteins with the membrane spanning region of Bcr⁵² (bicyclomycin resistance protein); PP_5173^{53,54} is a multidrug resistance efflux pump, TriABC-TolC which has been shown to be responsible for triclosan resistance of P. aeruginosa; PSF113_RS43745 is acriflavin resistance gene which maintains the concentration of this compound at lower concentration inside the cell. This compound is a basic dye which is sensed by the P. fluorescens strain in OSPW; PSF113_RS50665⁴⁹ is from the multidrug and toxic compound extrusion (MATE) family which mediates resistance to dyes, hydrophilic fluoroquinolones, and aminoglycosides; PSF113_RS35960 is AcrB/AcrD/AcrF family protein which has long been known to be involved in resistance to basic dyes, detergents, and antibiotics⁴⁹. Interference of toxic NA compounds with the electron transport systems causes increased production of hydrogen peroxide and other reactive oxygen species which can be removed by OxyR or NrdR regulons, superperoxide dismutases, peroxidases, putative catalase, alkyl hydroperoxide reductases, thiol peroxidases, and putative glutathione peroxidases. Surrogate substrate of expressed enzymes in upper pathway analysis revealed some potential chemical structures in OSPW (see FIG. 3C) that have been reported for the first time.

### i) General Pathways Analysis

KEGG pathway reconstructions was utilized to initially characterize the comprehensive physiological response of Pseudomonas spp. to environmental NA compounds mixture using RNA-seq results. This analysis elucidated the general response of the strains to NAs prior to identifying the degradation pathways and key enzymes. PS, FS, PC, and FC DEGs lists could be respectively assigned to 307, 402, 454, and 360 KO numbers.

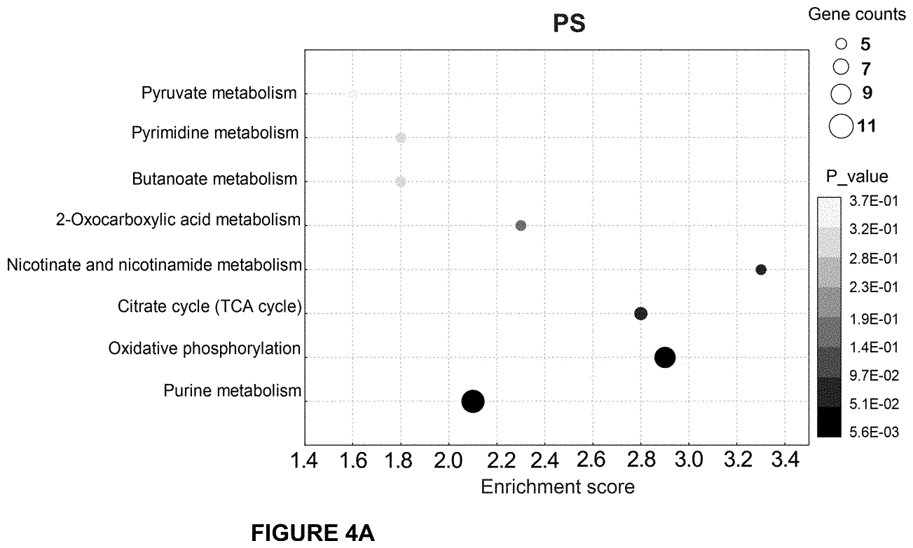

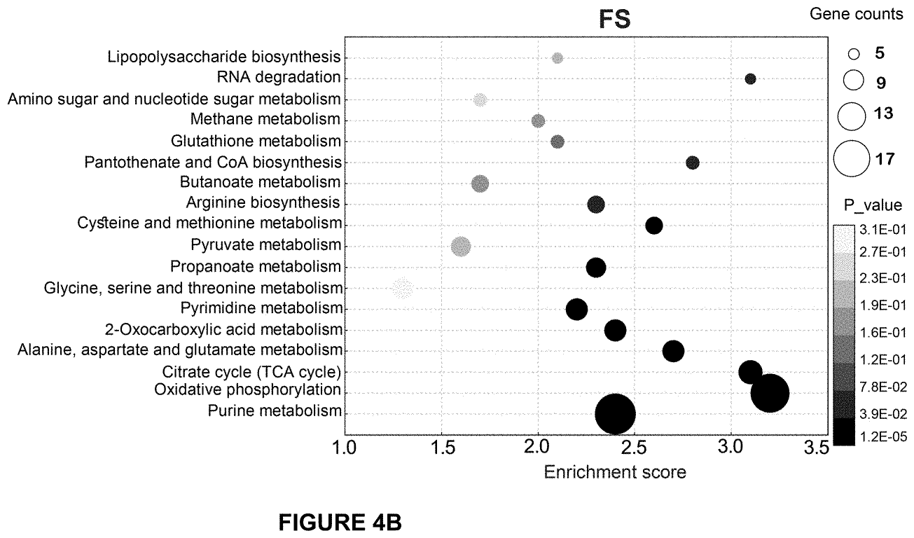

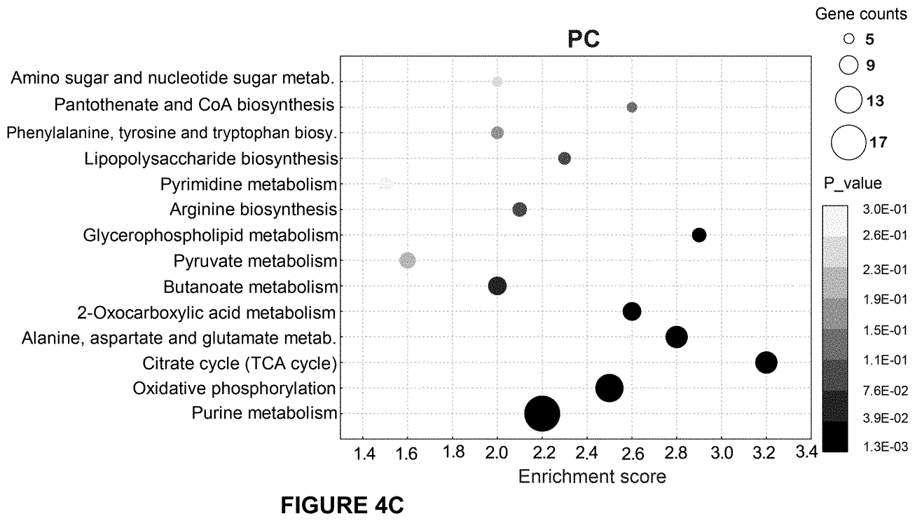

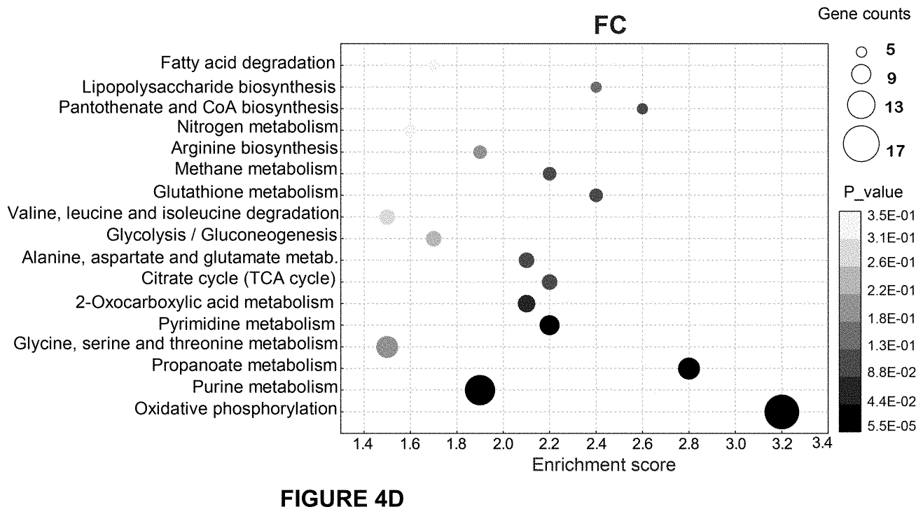

KEGG pathway enrichment analysis (FIGS. 4A-4D) showed that purine metabolism pathway is significantly enriched for all degradation samples. This pathway has been well-documented to play roles in antibiotic^{55,56}, acid and stress tolerance⁵⁷. It is suggested that modifications of flux through the purine nucleotide pathway and/or increased ppGpp concentrations provide a link between the physiological state of the cell and the level of stress tolerance and establish a role for the stringent response in acid stress response regulation.

The pathway involved in Nicotinate and nicotinamide metabolism is commonly significantly enriched (p-value=0.05) in PS. Two pathways have been identified in this map. The key genes upregulated for the first path is pncB nicotinate phosphoribosyltransferase. It is shown that exogenous niacin, that can be uptaken by aromatic compounds transporters, goes via niacin salvage pathway to generate NAD with the first enzymatic reaction 6.3.4.21 catalyzed by pncB gene product^{58,59}. Then, the highest expressed gene in PS expression browser was nicE, the last step in nicotinate degradation pathway, (maleate isomerase) catalyzed the reaction EC 5.2.1.1.

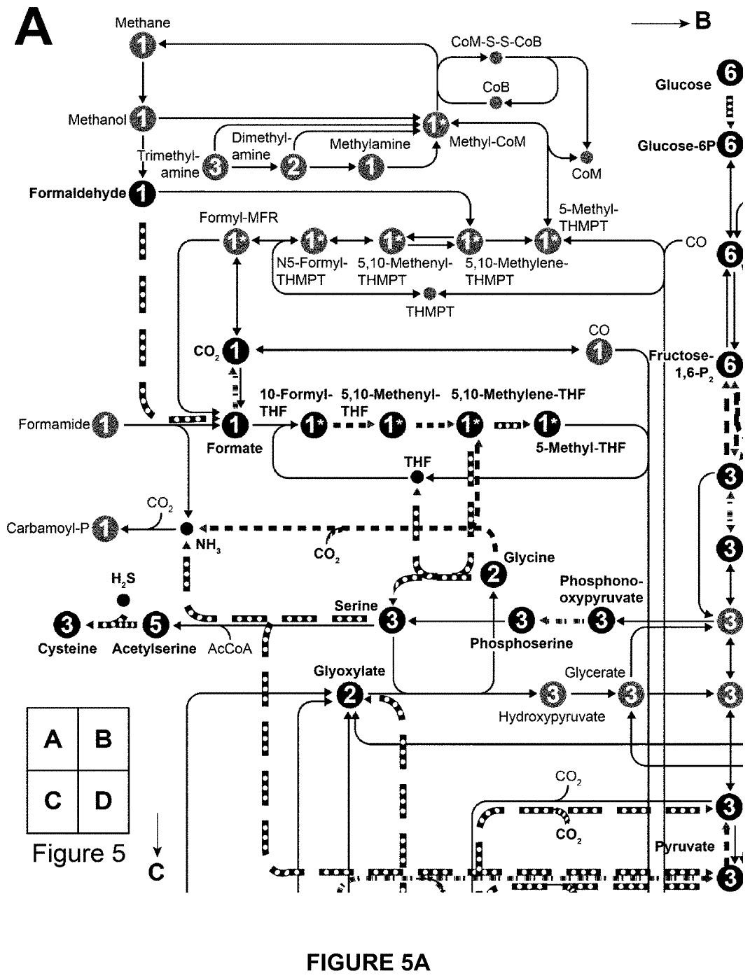

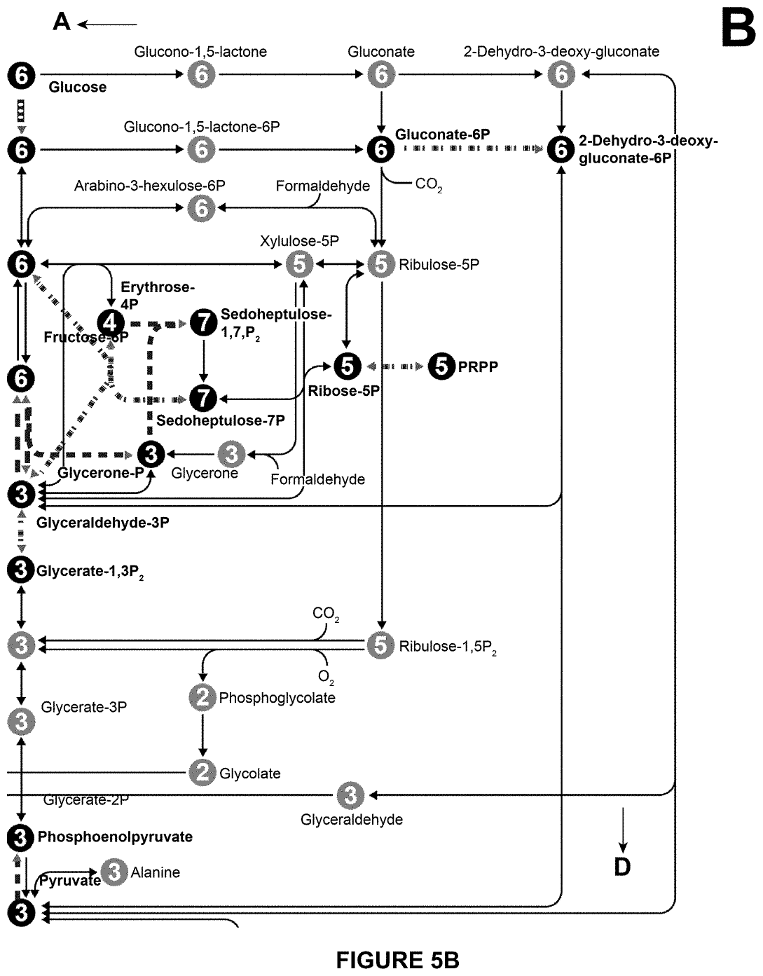

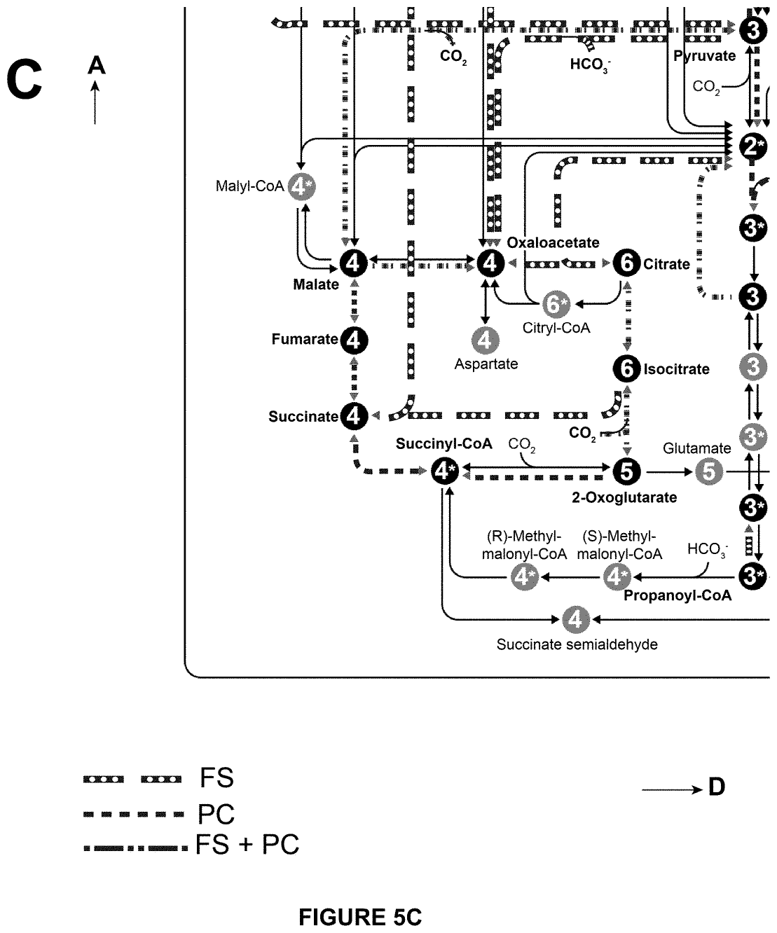

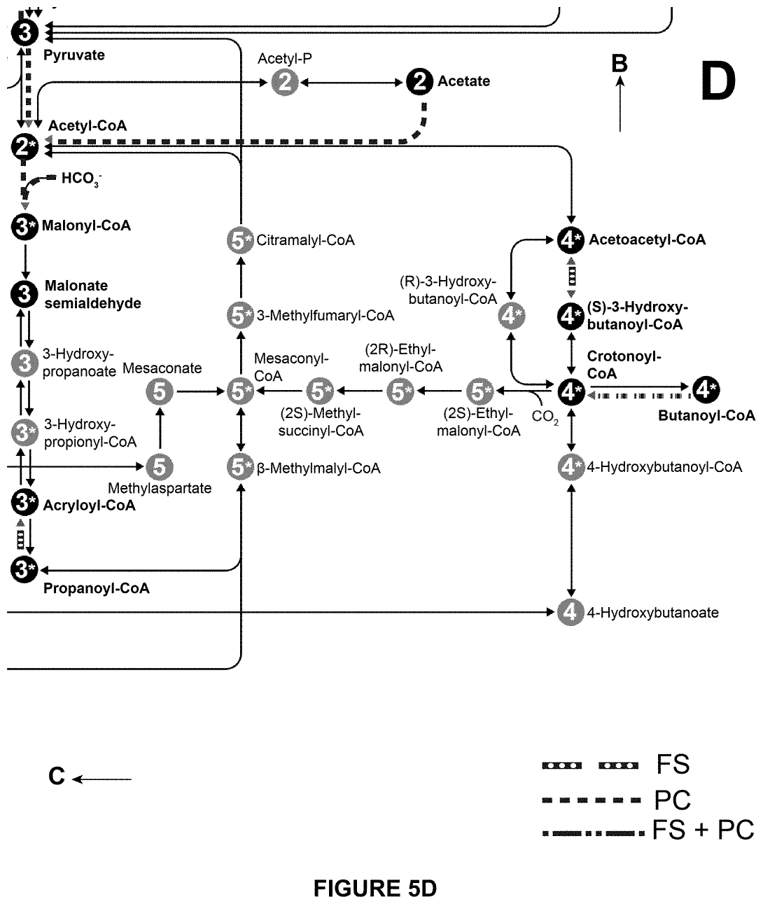

Maleate is a valuable dicarboxylic acid that is used to produce various polymer compounds and pharmaceuticals⁶⁰. Surprisingly, P. putida generated maleate when exposed to OSPW due to biodegradation of nicotinate in OSPW. While it was discovered that the exact mass 123.03 in ESI⁻ spectroscopy which can be associated to niacin existence in extracted OSPW, most of the nitrogen containing compounds might not be detected in negative ion mass spectroscopy. 2-oxocarboxylic acid metabolism pathway has been shown for all degradation samples while this pathway is significantly enriched for PC and FS with 9 and 10 gene counts, respectively. On this basis, the carbon metabolism global map of FS and PC were compared in FIGS. 5A-5D. It can be confirmed that only FS has significantly shunted isocitrate to glyoxylate using PSF113_RS41305 (isocitrate lyase), however, the further steps of glyoxylate cycle have not been differentially expressed. Since glyoxylate is toxic to this bacterium, it seems likely that a system is in place to retain it from accumulating.

Okubo et. al⁶³ has discussed an alternative route that consumes glyoxylate and converts it to glycine, methylene tetrahydrofolate, and serine which can be clearly observed in FIGS. 5A-5D for FS. However, the first reaction of this alternative route which is catalyzed by L-alanine-α-keto acid aminotransferases⁶⁴ (EC 2.6.1.-) has not been reconstructed in the map. Possible explanation for this observation might be the enzymes in transcriptomes of FS that could not be assigned to KO number. In FS degrading sample, the only differentially expressed aminotransferase which has not been assigned to KO number was PFS113_4986 pyridoxal phosphate-dependent aminotransferase catalyzed the reaction EC 2.6.1.1. General, as compared to PS, FS showed more versatile carbon metabolism routes from different terminal catabolites, significantly enriched pathways in aromatic heterocycles metabolism including purines and pyrimidines, and complete oxidative phosphorylation map (18 genes).

### j) NA Degradation Pathways Analysis

While the general pathways can be analyzed using KEGG pathways reconstructions, degradation pathways analysis with KEGG is impossible due to large pathway maps, incomplete KO assignment, and having no organism-specific maps. Here, we used P. putida KT2440 and P. fluorescens F113 PGDBs in Pathway Tools to reveal the NA biodegradation pathways from experimental RNA-seq data.

Compounds entering the degradation pathway can range from even-number saturated unbranched aliphatic (e.g., palmitic acid) to branched unsaturated chains, long-chain odd-number, alicyclic, aromatic acids as well as nitrogen or sulfur containing compounds with different terminal catabolites (acetyl-CoA, succinyl-CoA, propanoyl-CoA, etc.) transferring into distinct sections of central metabolism. Literatures mostly proposed β-oxidation pathways (FIG. 11) for commercial NAs (C_{c}H_{2c+z}O₂), however, biodegradation of complex environmental extracts (C_{c}H_{2c+z}NₙOₒSₛ) with unknown structures and fractions have not been characterized yet. The presence of a quaternary carbon at the α- or β-position or a tertiary carbon at the β- and β′-position, prevents β-oxidation and leads to dead-end compounds in the reaction sequence of this pathway (recalcitrant NAs).

Alicyclic NA compounds have been proposed to generate cyclohexane carboxylic acid (CHCA) and cyclohexane acetic acid (CHAA) as final metabolites depending on odd or even branched chains, respectively²⁰. Cyclohexane carboxylic acid and 4-methyl-CHCA have been predicted to metabolize through aromatization pathway by an Arthrobacter sp.⁶⁵ and Cupriavidus gilardii CR3 strains⁶⁶, respectively. Interestingly, we found Arthrobacter identity in entire microbial community characterization of OSPW with 0.29% abundance. The CHAA is predicted to first convert into CHCA via α-oxidation pathway and degraded further by β oxidation pathway²⁷.

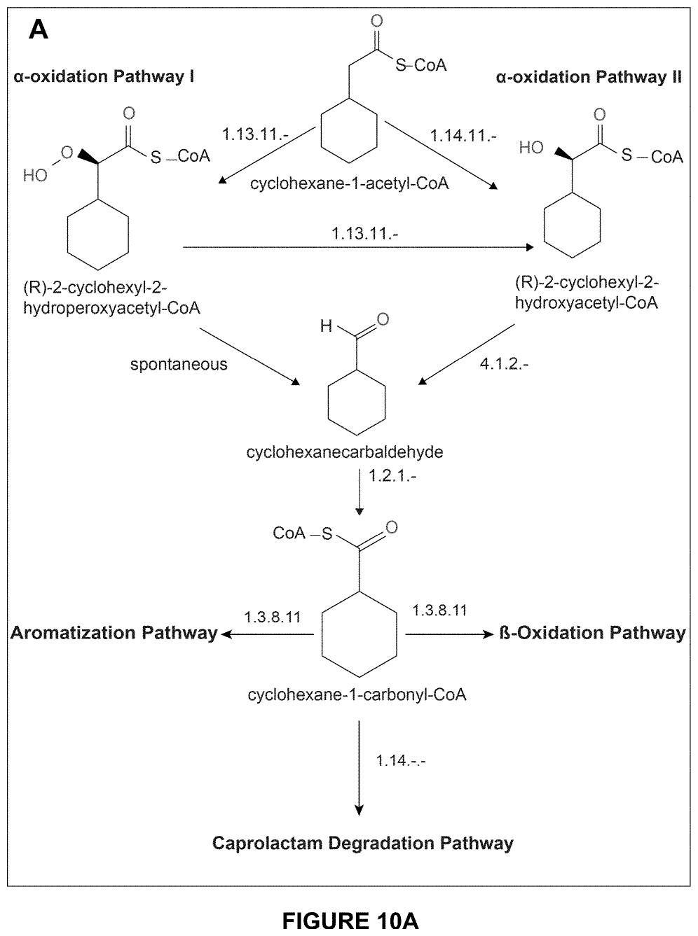

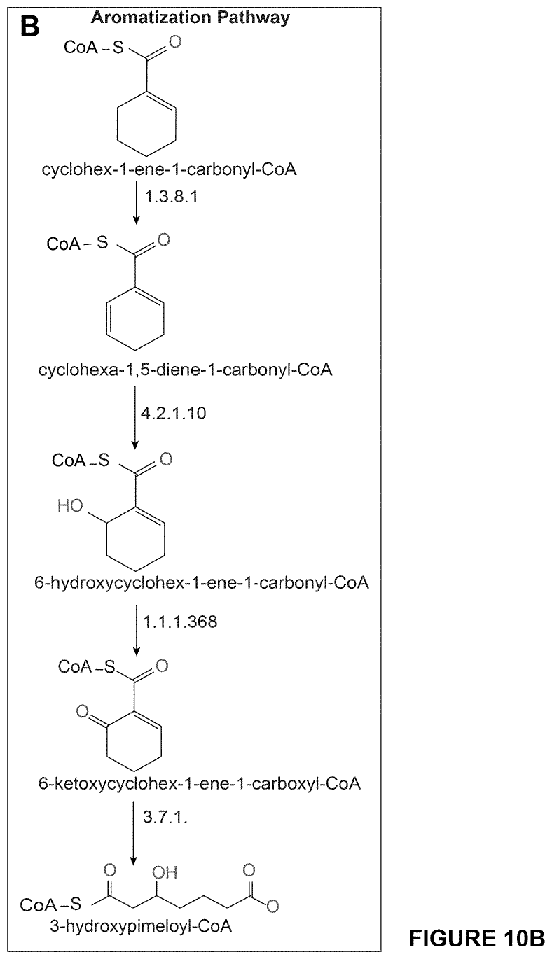

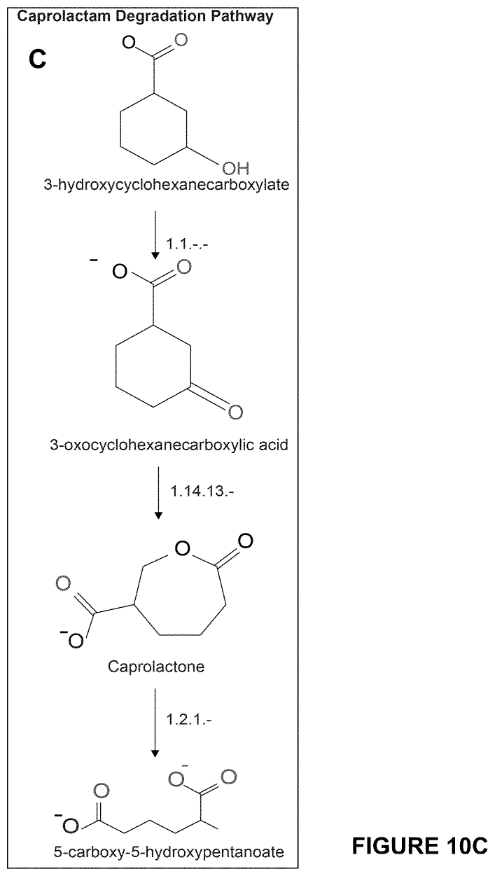

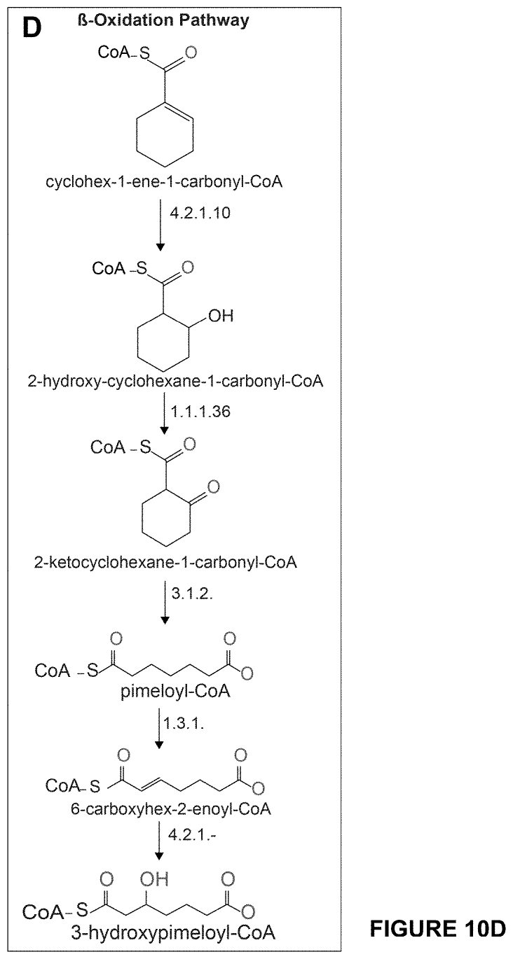

Cyclohexanone oxidation to adipic acid is proposed via a caprolactam degradation pathway in Brevibacterium epidermidis strain HCU⁶⁷ (FIGS. 10A-10D). While these pathways have been proposed in literature, the reactions of pathways and corresponding enzymes have not been discussed. The inventors, for the first time, comprehensively mined KEGG and MetaCyc databases and summarized available identified pathways for proposed alicyclic NA final metabolites (CHCC and CHAA) and summarized in FIGS. 10A-10D. Omega oxidation pathway for aromatic alkanoic commercial NAs by Mycobacterium spp²⁷ is also suggested.

To start the RNA-seq analysis for identification of degradation pathways, the inventors first looked at the previously reviewed β-oxidation pathways (FIGS. 10A-10D) in our RNA-seq data with four basic reactions acyl-CoA dehydrogenase (1.3.8-), enoyl-CoA hydratase (4.2.1.17), β-hydroxyacyl-CoA dehydrogenase (1.1.1.35), and acyl-CoA acetyltransferase or thiolase (2.3.1.16). For Pseudomonas putida samples, the identified DEGs in this pathway were acs (EC 6.2.1.3), gcdH (EC 1.3.8.-), and pcaF-II (EC 2.3.1.16). However, FS and FC degrading samples showed the complete β-oxidation pathway with the DEGs PSF113_RS36920 (EC 6.2.1.3), three acyl-CoA dehydrogenases catalyzing the reaction 1.3.8.-(PSF113_RS54825, PSF113_RS57765, PSF113_RS57780), PSF113_RS37405 (EC 4.2.1.17), PSF113_RS51245 (EC 1.1.1.35).

For unsaturated aliphatic NAs with cis configuration ((DBE=2) the additional reaction of enoyl-CoA isomerase PSF113_RS50625 (5.3.3.8) in P. fluorescens allowed re-entry of the intermediate into β-oxidation pathway. P. fluorescens strikingly showed the glutathione S-transferase (PSF113_RS49945) catalyzing the reaction 2.5.1.18 in metabolism of xenobiotics by cytochrome P450 pathway.

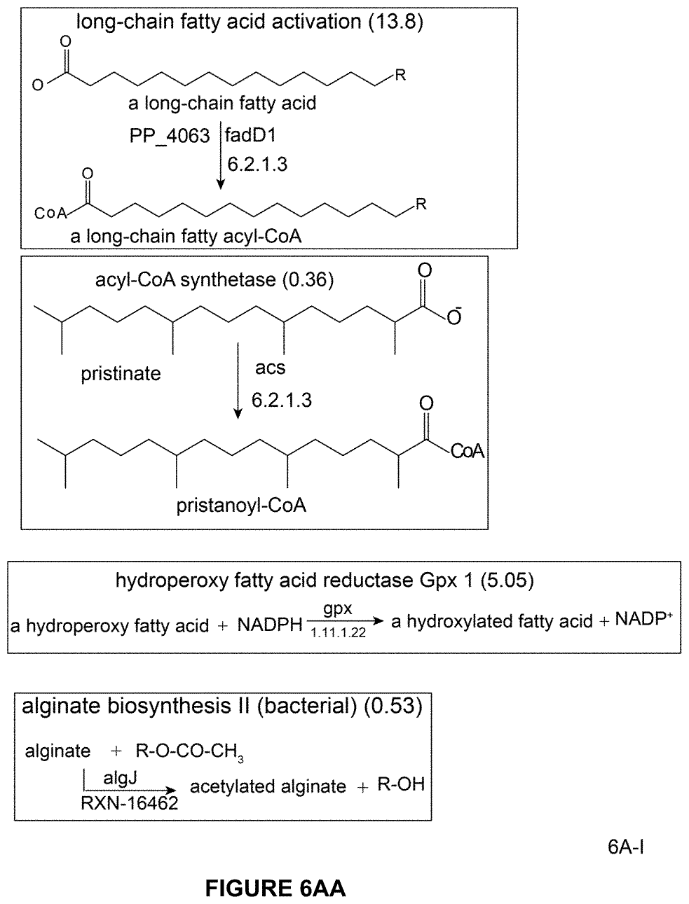

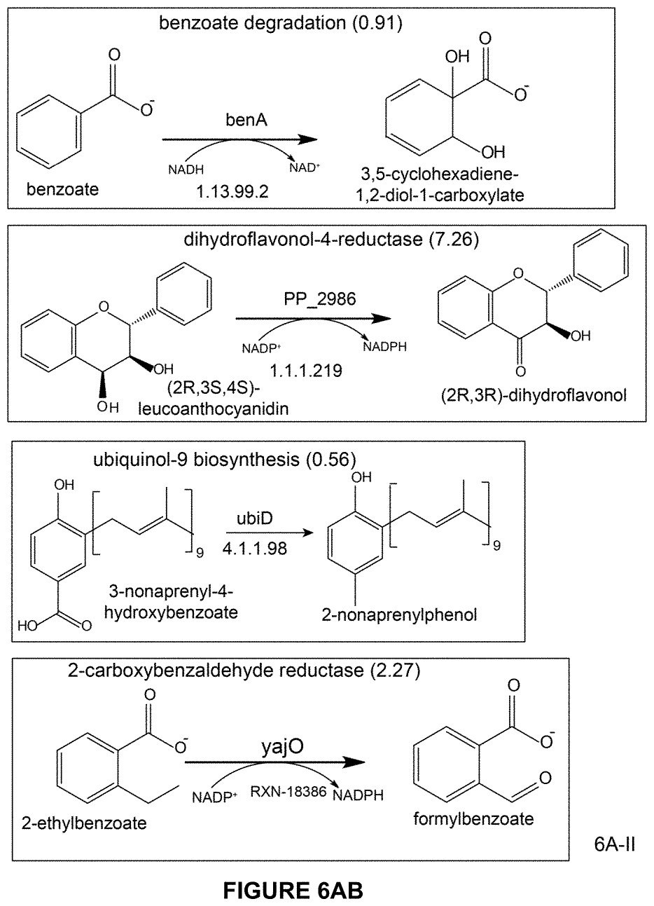

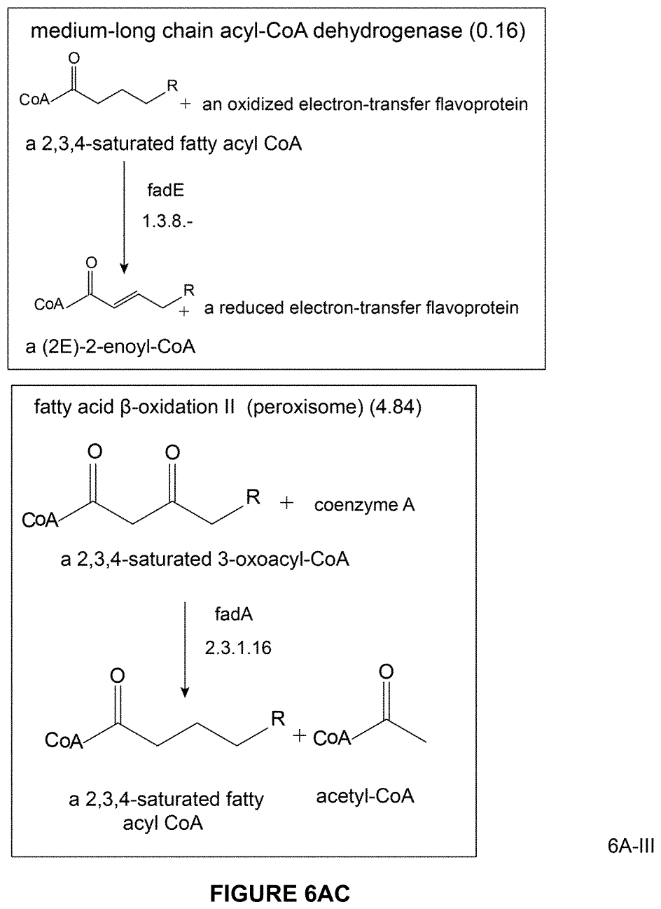

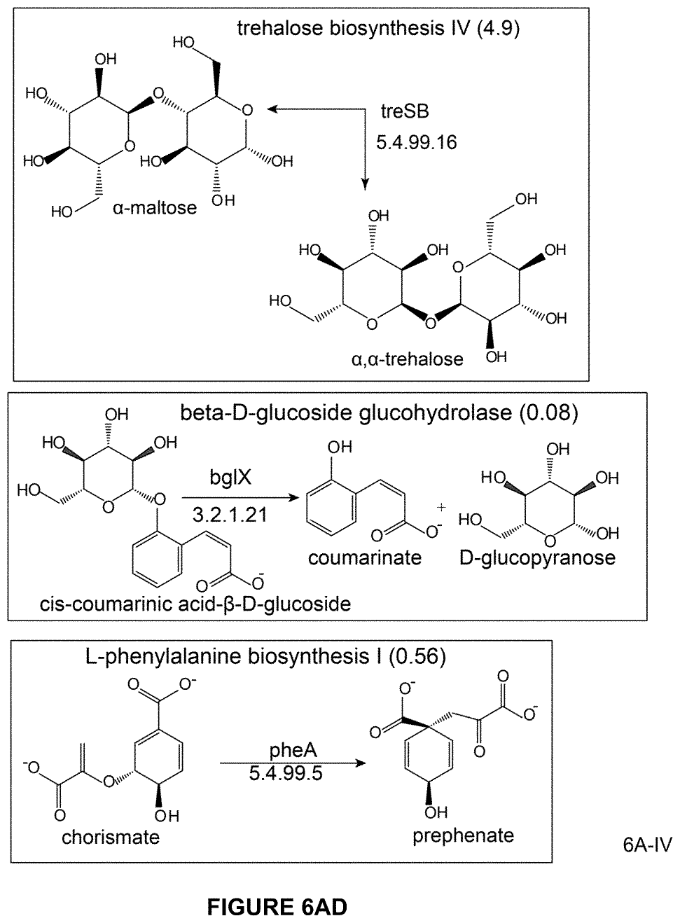

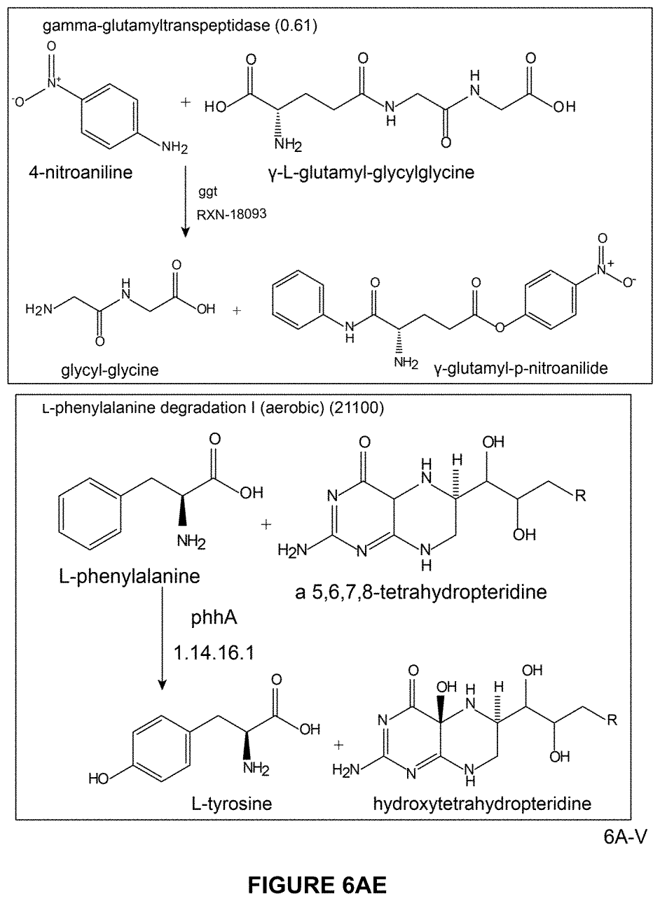

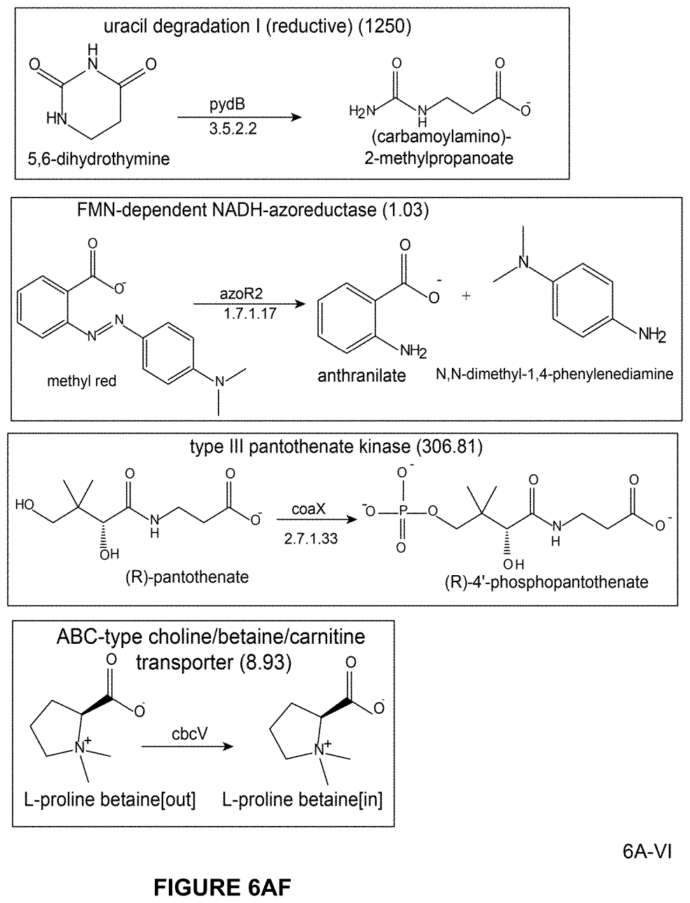

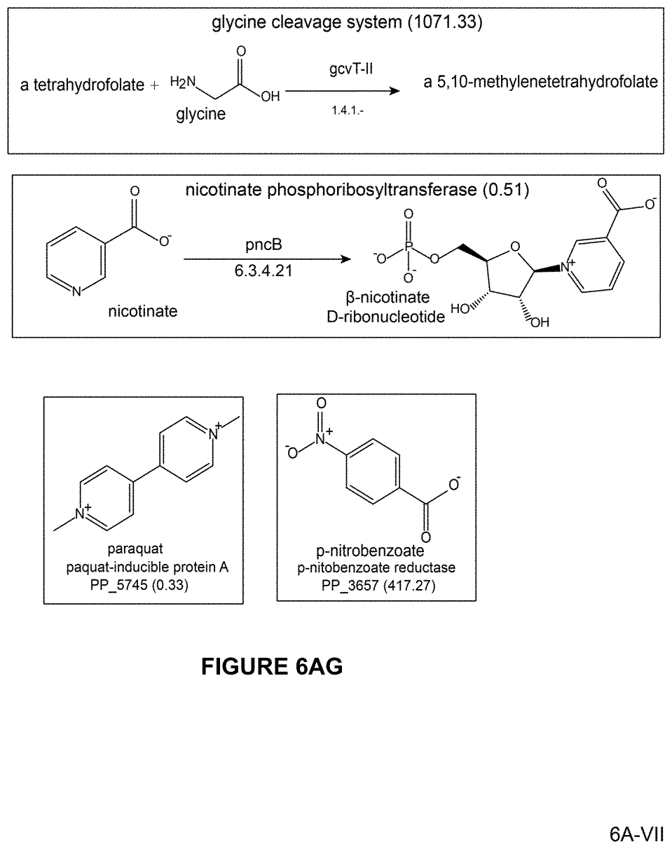

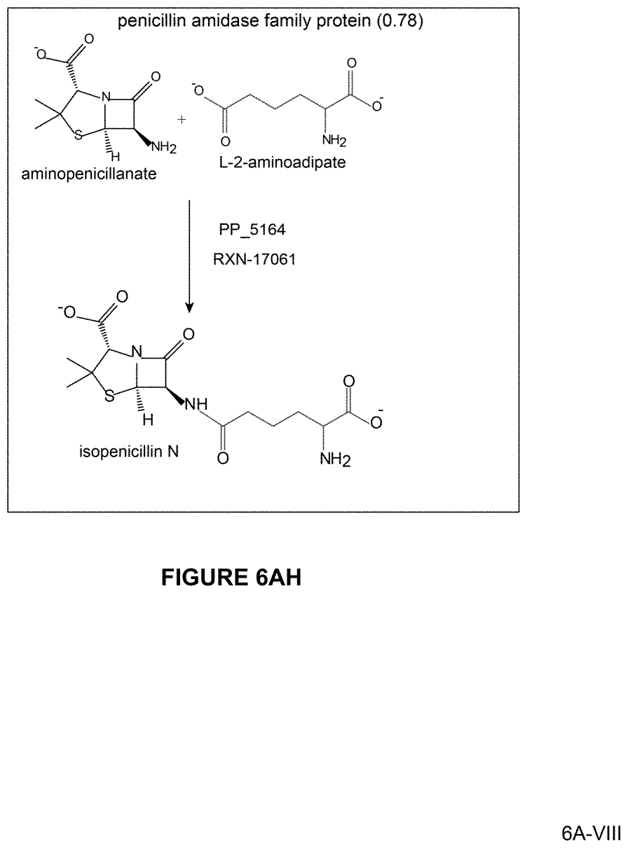

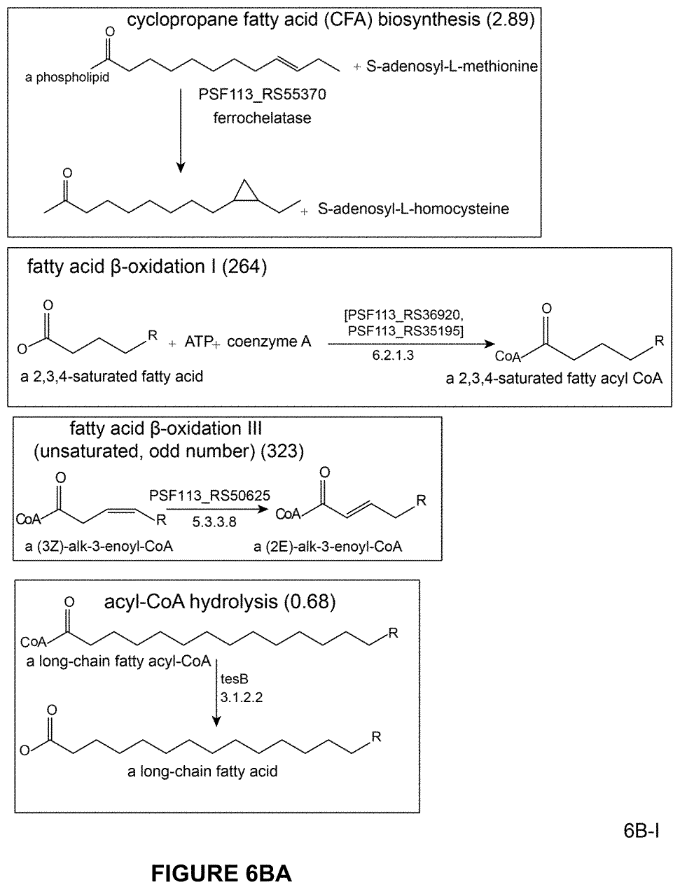

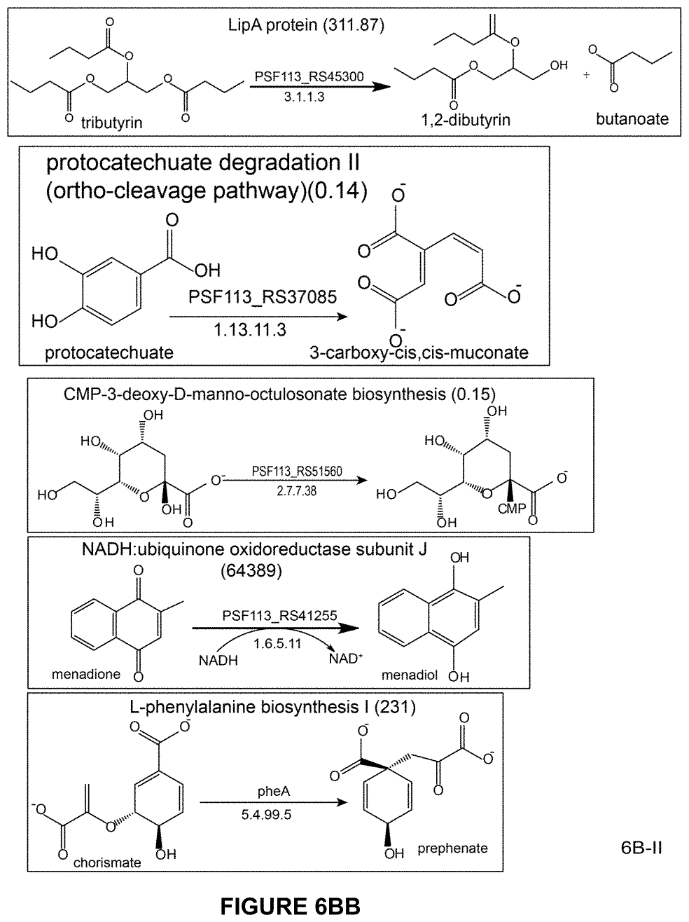

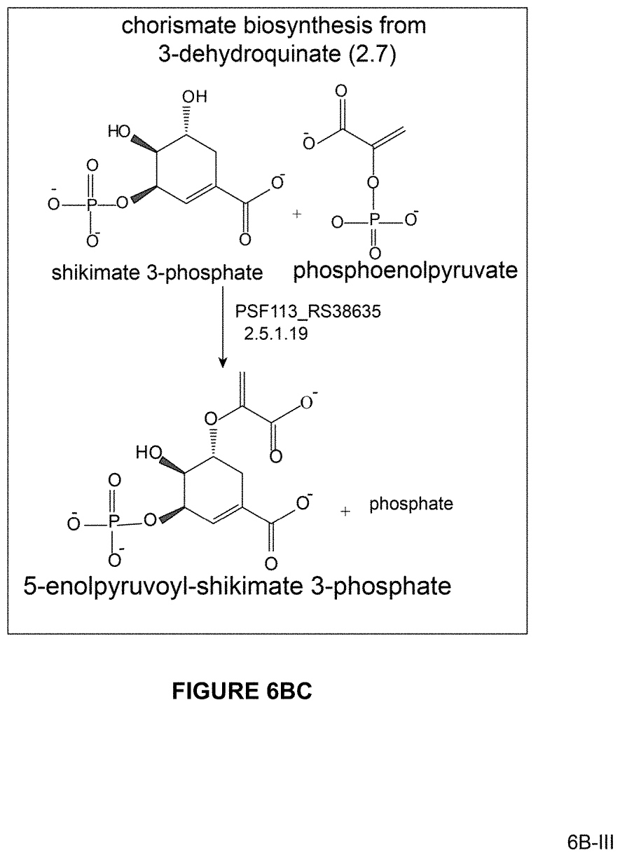

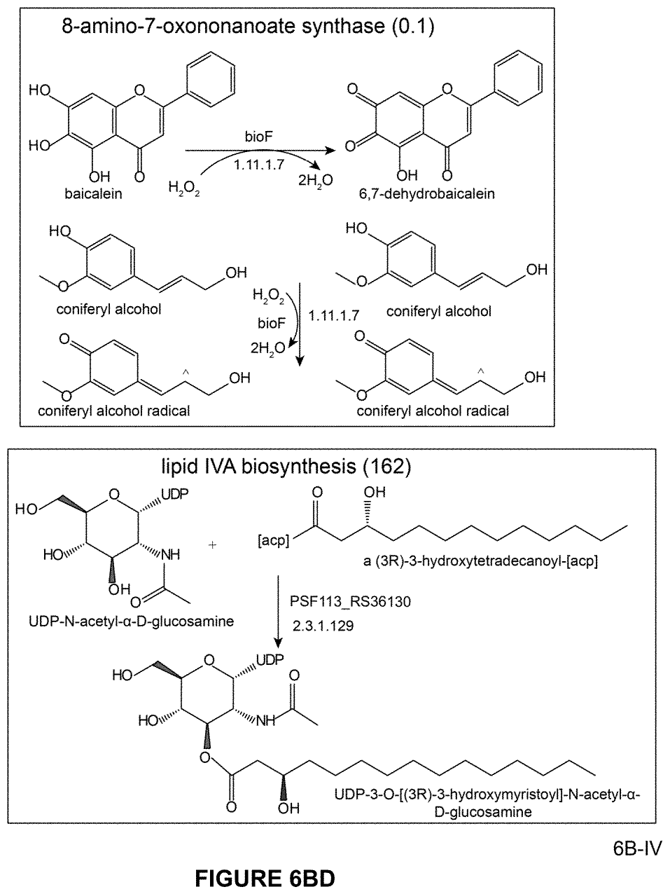

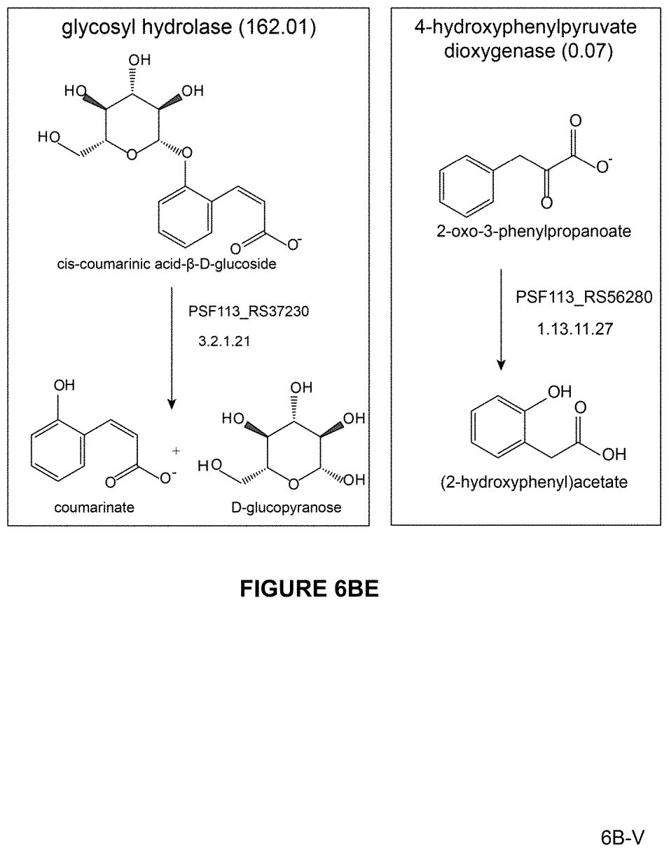

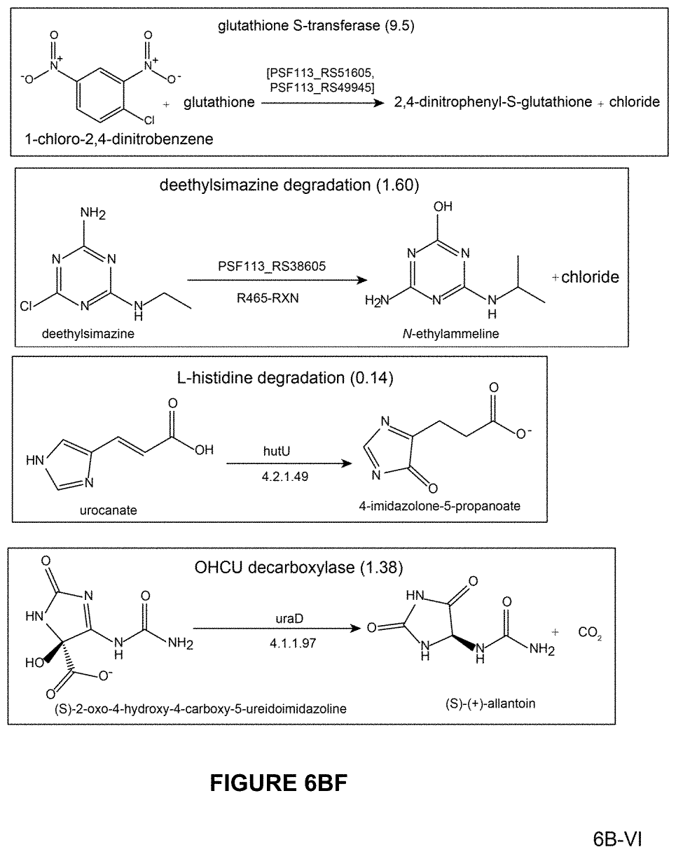

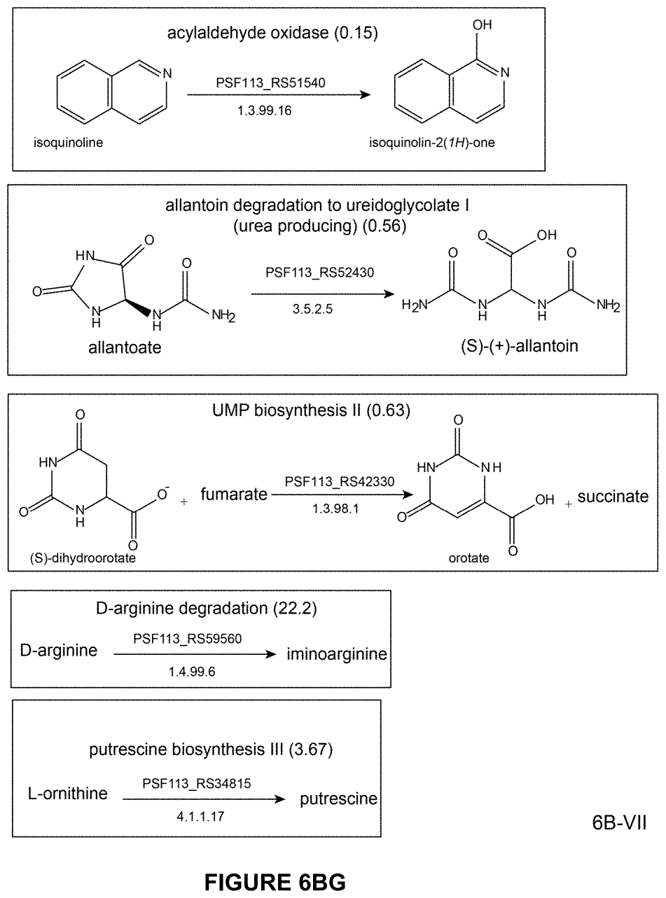

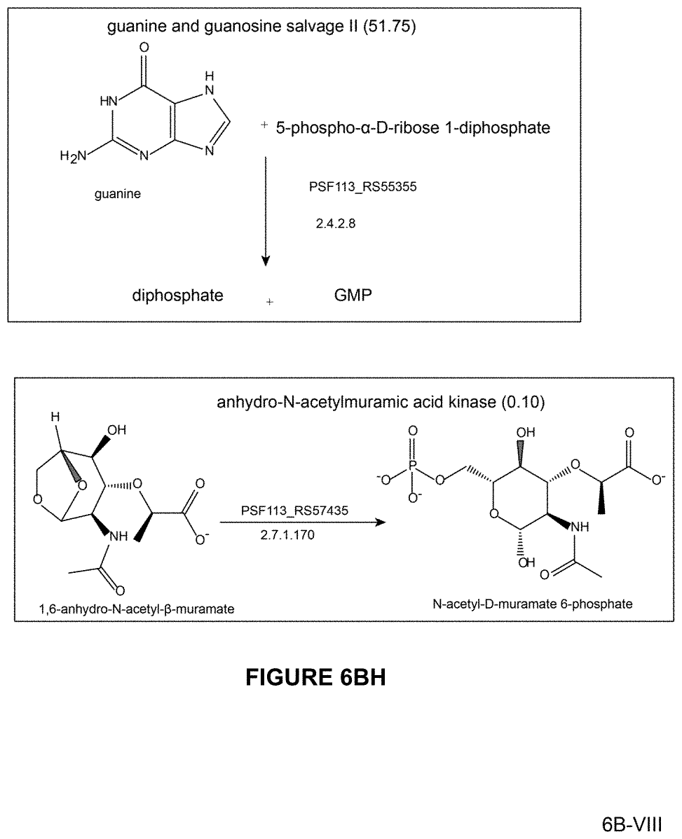

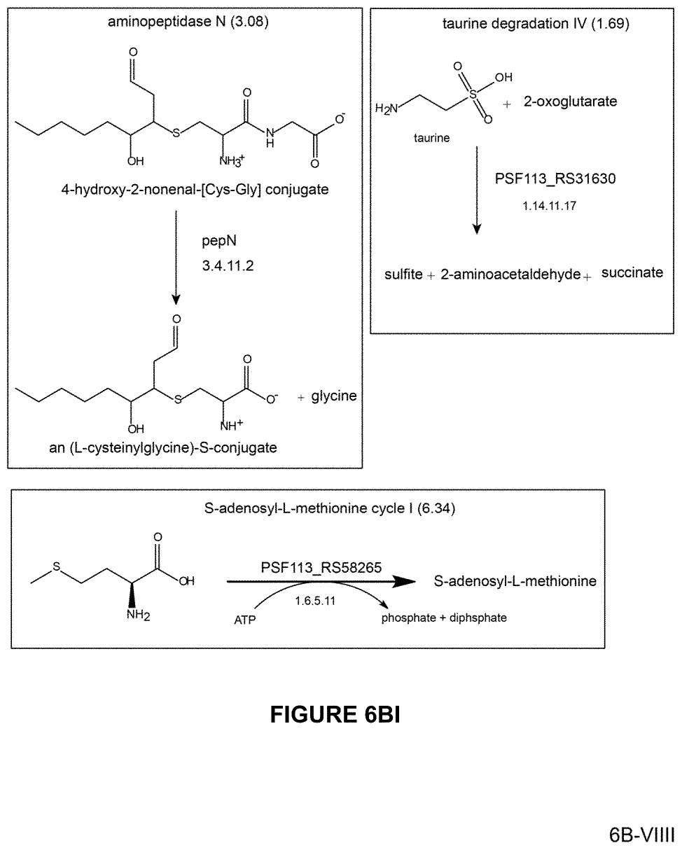

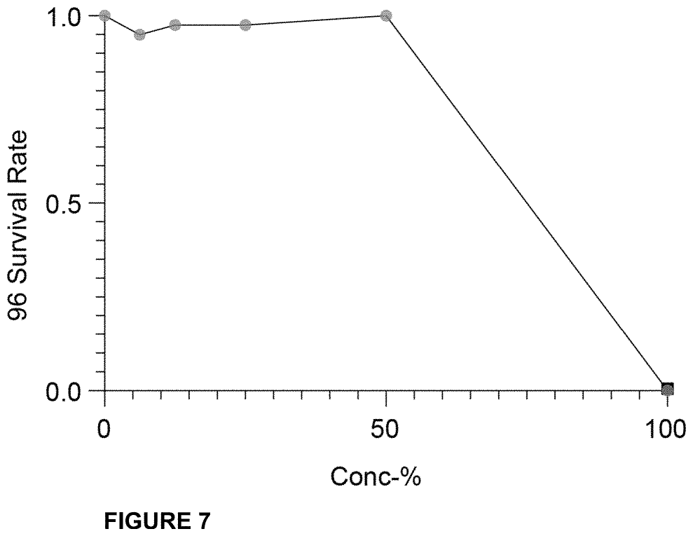

The analysis of DEGs by Pathway Tools revealed the pathways by which different range of naphthenic acids have been degraded and used for cellular division (growth linked biodegradation) or assimilated into biosynthetic pathways but remain unable to support cellular division⁶⁸ (fortuitous oxidation). FIGS. 6AA-6AH and FIGS. 6BA-6BI shows the identified degradation pathways and the abundance of the pathways quantified by Pathway Perturbation Score (PPS)⁶⁹. The PPS measures the overall extent to which a pathway is up-regulated, by combining the activation levels of all reactions in the pathway.

A Reaction Perturbation Score (RPS) is computed for each reaction as the maximum absolute value of all data values for differentially expressed genes associated with the reaction. To compute the PPS, we sum the squares of the RPSs for all reactions in the pathway for which data are available, divide by the number of reactions for which data are available, and take the square root of the result (we use the square of the RPSs instead of a traditional average in order to weight larger RPSs more heavily). For a pathway containing N reactions: PPS=sqrt [(RPS₁²+RPS₂²+ . . . +RPS_{N}²)/N].

The identified degradation pathways, the corresponding logarithm of PPS, pathway input and terminal catabolites have been shown in FIGS. 6AA-6AH and FIGS. 6BA-6BI.

A total of 26 reactions were identified by matching their substrates to the molecular formulas of NAFCs in HPLC-Orbitrap results in P. putida (PS) (FIG. 6AA-6AH). In general, pathways that metabolize O compounds in NAFCs showed lower activity and variety in PS cultures. Also, the PS cultures uniquely expressed pathways for alginate biosynthesis (bacterial type II), benzoate degradation, trehalose biosynthesis (type IV), and ubiquinol-9 biosynthesis, which suggests that the PS and FS cultures use different mechanisms to degrade NAFCs. The PS cultures also showed unique reactions for degradation of substrates such as dihydroflavonol (1.1.1.219, catalyzed by dihydroflavonol-4-reductase) and branched fatty acids such as pristanate (6.2.1.3, catalyzed by acyl-CoA synthetase). The pathways for L-phenylalanine biosynthesis I and fatty acid β-oxidation were common between PS and FS cultures. However, the number and diversity of enzymes in pathways that metabolize O compounds, including long-chain fatty acid activation, and fatty acid β-oxidation II, is significantly higher in PS cultures. The degradation of N and NO NAFC compounds by the PS cultures principally occurs via 4 pathways, namely uracil degradation I (reductive), L-phenylalanine degradation I (aerobic), folate transformations I, and phosphopantothenate biosynthesis I. There were also 5 genes with the reactions that could be assigned to N and NO substrates without a characterized pathway. These substrates are para nitrobenzoate, paraquat, methyl red, α-glutamyl-p-nitroanilide, nicotinate and L-proline betaine. The PS cultures also express penicillin amidase proteins to metabolize NOS compounds.

A total of 36 reactions have been identified by matching their substrates to the molecular formulas of NAFCs in HPLC-Orbitrap results in P. fluorescens (FS) (FIG. 6BA-6BI). In the biodegradation of 0 compounds in NAFCs, FS culture showed 22 reactions or pathways. Fatty acid fatty acid β-oxidation III (unsaturated, odd number) showed the highest pathway perturbation score (PPS) 323 that PSF113_RS50625 gene product 3-hydroxyacyl-CoA dehydrogenase were shown to have substrates potentially related to NAFCs compounds. When fatty acids contain a cis-double bond on odd-numbered carbons, these compounds must be first converted to a (2E)-2-enoyl-CoA before they can be processed further by β-oxidation I pathway (PPS=264). L-phenylalanine biosynthesis (PPS=231) begins with the conversion of chorismate to prephenate catalyzed by the chorismite mutase the product of pheA. Enrichment of this pathway suggested that some mono cyclic compounds of NAFCs might be used to generate aromatic amino acids in these cultures. Other than β-oxidation pathways, lipid IVA biosynthesis has been also enriched with the substrate of fatty acids in the form of a (3R)-3-hydroxytetradecanoyl-[acp] that is linked to UDP-N-acetyl-α-D-glucosamine to form the lipid IVA intermediate in the lipid A biosynthetic pathway. Glyoxylate shunt—or, to be more precise, the step catalyzed by isocitrate lyase—is also upregulated in FS culture suggesting the versatility of catabolic routes for utilizing NAFCs as a source of carbon.

Cyclopropane fatty acid (CFA) biosynthesis is upregulated in the FS and FC cultures. CFA synthase is a key enzyme in the pathway and methylenates the acyl chains of unsaturated fatty acids to their cyclopropane derivatives. The expression of this enzyme indicates that the degradation products of NAs may be eventually feeding into lipid biosynthesis, which is consistent with the observation lipid metabolism GO term in FS culture. Enrichment of chorismate biosynthesis from 3-dehydroquinate pathway indicates that cyclic structures of NAFCs (structures like 3-dehydroquinate) might be converted to chorismite that is an intermediate in the synthesis of the three aromatic amino acids: L-phenylalanine, L-tyrosine and L-tryptophan. Acyl-CoA thioesterase (tesB gene product) hydrolyzes fatty acyl-CoAs to free fatty acids supporting growth on fatty acids or conjugated fatty acid as the sole source of carbon in acyl-CoA hydrolysis pathway (PPS=0.68).

In CMP-3-deoxy-D-manno-octulosonate biosynthesis, the last reaction (2.7.7.38) is upregulated converting α-2-keto-3-deoxyoctulosonic acid pyranose (Kdo) to the activated form of CMP-Kdo that is the actual donor of Kdo units for synthesis of inner core of lipopolysaccharides in outer membrane of gram-negative bacteria. 2-methylcitric acid cycle is the propionate degradation pathway that the Ca methylene group of propionate is oxidized to a keto group yielding pyruvate, a common precursor for biosynthesis and energy production. Protocatechuate degradation II (ortho-cleavage pathway) is a well know pathway in degradation of various aromatic compounds, and part of 3-oxoadipate pathway that are both enriched in FS cultures. Biodegradation of a variety of aromatic compounds could generate protocatechuate, key intermediate metabolites, that is then converted to succinyl-CoA and acetyl-CoA in 3-oxoadipate pathway, to be processed by TCA cycle. As for single reactions that showed matching compounds in the RNA-seq data, the highest RPKM belonged to PSF113_RS41260 gene product catalyzing the generation of menadiol from menadione with EC:1.6.5.11. With expression of PSF113_RS45300 gene, tributyrin could be converted to 1,2-dibutyrin and butanoate catalyzing by a triacylglycerol lipase (EC: 3.1.1.3). Other expressed enzymes for this class of NAFCs includes 3-phosphoshikimate 1-carboxyvinyltransferase, glycosyl hydrolase, 4-hydroxyphenylpyruvate dioxygenase, 5-carboxymethyl-2-hydroxymuconate semialdehyde dehydrogenase, lipoyl synthase, and 8-amino-7-oxononanoate synthase that all the substrates and reactions are shown in FIGS. 6BA-6BI.

For N and NO compounds in NAFCs, 7 pathways have been shown in FIG. 6BA-6BI including guanine and guanosine salvage, guanine and guanosine salvage II, D-arginine degradation, putrescine biosynthesis III, deethylsimazine degradation, UMP biosynthesis II, allantoin degradation to ureidoglycolate I (urea producing), and L-histidine degradation. In addition to the characterized pathways, this culture showed 4 reactions that their substrates were 1-chloro-2,4-dinitrobenzene, isoquinoline, (S)-2-oxo-4-hydroxy-4-carboxy-5-ureidoimidazoline, and 1,6-anhydro-N-acetyl-β-muramate. For NOS compounds of NAFCs, on the other hands, FS showed S-adenosyl-L-methionine cycle I and taurine degradation IV pathways, and expression of an aminopeptidase N catalyzing the reaction of 3.4.11.2.

### k) Toxicity Measurements for NA Compounds Mixtures

The 96 h LC50 (Fathead Minnow Embryo Lethality) and EC50 (15 minutes of exposure to Vibrio fischeri) were measured for extracted OSPW at nominal concentration of 1× naphthenic acids reconstituted in DI water and raw OSPW according to the protocol of Morandi et al.². Only EC50 was measured for the degradation samples.

Extracted OSPW at nominal concentration of 1× showed acute toxicity toward embryos of fathead minnow (LC50) and Vibrio fischeri (EC50). The 96 LC₅₀ and EC₅₀₋₁₅ minutes (FIGS. 8A-8E) of extracted OSPW at nominal concentration of 1× naphthenic acids mixture were 70.1 and 82%, respectively. For Pseudomonas putida and fluorescens degradation samples, the toxicity decreased with increasing the EC50 to 92.1 and 94.3%, respectively.

### I) Methods and Means for Generating and Analyzing Gene Expression Profiles

In one aspect, the present invention is directed to a method for generating gene expression profiles of a microorganism isolated from an environment contaminated with toxic organic compounds. The method comprises: a) preparing RNA samples from a first microorganism strain grown i) in the presence of naphthenic acid and ii) in the absence of naphthenic acid; b) preparing amplicon cDNA libraries of differentially expressed genes using Suppression Subtractive Hybridization (or similar methods) of the RNA samples from i) and ii); c) determining the relative frequency of expression of RNA for the microorganism by sequencing the amplicon cDNA libraries; d) constructing an in silico genome assembly from the cDNA libraries; annotating the in silico genome assembly to predict differentially expressed genes; and e) determining the number of reads for each of the differentially expressed gene.

In one embodiment, the method comprises preparing the RNA samples from a second microorganism strain grown individually i) in the presence of naphthenic acid and ii) in the absence of naphthenic acid (NA), and grown in combination with the first microorganism.

The RNAseq libraries can be prepared using, for example, Suppression Subtractive Hybridization of the total isolated RNA pools from the microorganisms (e.g., P. putida and P. fluorescens) as grown individually, as well as together in the presence of raw and extracted naphthenic acids (NA). Details of the Suppression Subtractive Hybridization involves the following steps:

1) Purifying the total RNA from the microorganism in the presence of extracted naphthenic acid (NA) from OSPW (i.e., positive control).

2) Purifying the RNA from the microorganism in the absence of extracted naphthenic acid (NA) (i.e., negative control).

3) Applying Suppression Subtractive Hybridization to the RNA extracted in steps 1 and 2 to generate three amplicon cDNA libraries of differentially expressed genes in the microorganism samples. The cDNA libraries can be used to infer the unique transcriptomic signatures of the strains when they are exposed to naphthenic acids.

4) Determining the relative frequency of expression of the plurality of the total RNA for the microorganism using next-generation sequencing (NGS) of the amplicon cDNA libraries as prepared in step 3.

The process for constructing and annotating the in silico genome assembly from a RNA seq library are set out below.

1) Raw reads are processed through Trimmomatic to remove adapter sequences, sequence ends with poor base-qualities, and entire reads which are of poor quality.

2) Digital normalization using the Khmer software is performed to remove sequencing reads redundant beyond 255-fold coverage as well as reads containing low-abundance k-mers with minimum abundance threshold of 20.

3) Transcriptome assembly is accomplished using rnaSPAdes (de novo Genome Assembly Method) with k=55, no mismatch correction and no coverage cut-off as these are normalized data.

4) Prior to open reading frame (ORF) predictions, the assembled genome database is processed using MetaPathways pipeline to renames input contigs with standardized identifiers and removes any contigs smaller than 180 base pairs.

5) MetaPathways is used to predict the ORFs. Predicted ORFs less than 180 base pairs or 60 amino acids in length are removed by default form downstream analysis to achieve more reliable alignment and annotation outcomes (Length: 180 bp (60 aa)), E-value: 1e-6, bit-score: 20, Bit-score ratio >0.4. Pseudomonas putida KT2440 and Pseudomonas fluorescens F113 are used as reference genome in Metacyc annotation.

6) Three differentially genes expression browsers of Pseudomonas putida, Pseudomonas fluorescens and both are generated after annotations and with the reads per kilobase of transcript per million (RPKM) for each differentially expressed genes.

In another aspect, the present invention is directed to a method for identifying genetic element involved in microorganism adaptation to toxic organic compounds. The method comprises: a) generating reconstructed metabolic pathways using gene expression profiles; b) enriching the reconstructed metabolic pathway and assigning an enrichment score; and c) analyzing the internal transport and activation and initiation of degradation, through functional gene clustering of the gene expression profiles, to identify a genetic element that are activated upon exposure of the microorganism to toxic organic compounds.

By “adaptation”, it is intended to mean the microorganism's ability to adapt to changing conditions such as, for example, oxidative stress, temperature challenges and osmotic perturbations. For example, the inventors reconstructed and enriched general metabolic pathways to analyze the adaptation strategies and physiological responses of microorganism (e.g., P. putida, P. fluorescens and both) in response to exposure to naphthenic acids. In particular, the physiological response and adaptation strategy analysis involves:

1) Converting the gene expression profiles to Entrez Gene ID profiles using Pathway Tools Smart Tables.

2) Assigning KO numbers to Entrez Gene ID profiles using Uniport Retrieve/ID mapping tool (www.uniprot.org) and general metabolic pathway are reconstructed by KEGG mapper tool from KO assigned expression profiles.

3) Enriching the reconstructed pathways and assigning an enrichment score to each reconstructed pathway using David Bioinformatics software, where threshold of gene counts and the Expression Analysis Systematic Explorer (EASE) score are set at 5 and 0.4, respectively.

4) Analyzing the enriched pathways from step 3) for each expression profiles determine the adaptation strategy and the physiological response of the corresponding sample in exposure to naphthenic acids (NA).

5) Analyzing upper pathways, which collectively describe the internal transport, activation and initiation of degradation, through functional gene clustering of the Entrez Gene profiles from step 1 using David Bioinformatics.

6) Identifying out membrane proteins, the ATP-binding cassette (ABC) transporters, major facilitator superfamily (MFS) transporters, efflux pumps, chaperons, regulator elements, chemotaxis elements, active oxygen species (AOS) responsive genes, and secretion signaling elements that are selectively induced upon exposure of the strains to naphthenic acids (NA).

In another aspect, the present invention is directed to a method of identifying genetic elements involved in a microorganism degradation of toxic organic compounds, comprising: overlaying gene expression profiles onto a pathway-genome database; and identifying pathways related to degradation of toxic organic compounds to determine enzymes involved in the pathway. For example, the present method can analyze the naphthenic acid (NA) degradation pathways by overlaying the gene expression profiles on to a microorganism (e.g., P. putida KT2440 and P. fluorescens F113 Pathway Genome Databases (PGDB) using Omic Viewer Tool in Pathway Tools. As a result, the degradation pathways related to naphthenic acid (NA) are identified from reconstructed Table of Pathways Diagrams.

It will be understood that genes that can not be characterized in the aforementioned method but may be putatively related to naphthenic acid (NA) adaptation and/or degradation are categorized through functional clustering of gene expression profiles. For instance, these genes are categorized into either possessing oxidoreductase activity or possessing oxidoreductase domain classes using the David Bioinformatics software.

In an embodiment, the method further comprises clustering a gene from the gene expression profiles not identified in a pathway to determine its function.

In another embodiment, the identified genetic element in the method is an enzyme for naphthenic degradation in Pseudomonas selected from the group consisting of: catalase (katA), alkyl hydroperoxide reductase subunit F (ahpF), thioredoxin reductase (trxB), peroxiredoxin (tsaA), hydroperoxy fatty acid reductase Gpx1 (gpx), multidrug transporter membrane protein (mdtB), fusaric acid resistance protein (PP_1266), RND transporter membrane fusion protein (PP_3301), multidrug transporter membrane protein (mdtC), multidrug RND transporter MexF (mexF), RND family transporter (PP_5173), Bcr/CflA family multidrug resistance transporter (PP_3304), RND family transporter (PP_3302), uronate transporter (PP_2837), glucarate transporter (gudP), transporter (PP_3250), tartrate MFS transporter (PP_3391), MFS transporter (PP_3566), metabolite transport protein YhjE (yhjE), carbohydrate efflux transporter (yhhS), aromatic compound MFS transporter (PP_3658), acyltransferase (PP_1700), TonB-dependent receptor (PP_3340), porin F (oprF), peptidoglycan-associated lipoprotein (oprL), phenylacetic acid-specific porin (phaK), outer-membrane porin E (oprE), outer-membrane porin D (oprQ), outer membrane protein assembly factor (bamA-II), outer membrane protein assembly factor (bamA-I), outer membrane ferric citrate porin (fecA), OmpA family protein (PP_4198), LPS-assembly protein LptD (IptD), lipid A 3-O-deacylase (pagL-I), glycine-glutamate dipeptide porin (opdP), hypothetical protein (PP_4115), ferrioxamine receptor (PP_0160), ferrichrome-iron receptor (PP_4755), ferric siderophore receptor (PP_3330), ferric siderophore receptor (PP_3325), aromatic compound-specific porin (PP_3656), ferric siderophore receptor (PP_0535), alginate production protein AlgE (algE), alginate biosynthesis protein AlgK (algK), thioredoxin (PSF113_RS59500), thioredoxin (PSF113_RS59220), thioredoxin reductase (PSF113_RS41395), stringent starvation protein A (PSF113_RS54790), peroxidase (PSF113_RS53615), peroxiredoxin (PSF113_RS39210), glutathione peroxidase (PSF113_RS55240), glutathione peroxidase (PSF113_RS39005), catalase (PSF113_RS52605), catalase (PSF113_RS57160), alkyl hydroperoxide reductase subunit F (PSF113_RS42115), secretion protein HylD (PSF113_RS36900), RND transporter MFP subunit (PSF113_RS54645), RND efflux transporter (PSF113_RS35960), multidrug transporter MatE (PSF113_RS50665), multidrug efflux RND transporter permease subunit (PSF113_RS44170), multidrug ABC transporter substrate-binding protein (PSF113_RS50525), acriflavine resistance protein B (PSF113_RS43745), MFS transporter (PSF113_RS53065), MFS transporter (PSF113_RS50550), 4-hydroxybenzoate transporter (PSF113_RS37090), MFS transporter (PSF113_RS38620), TonB-dependent receptor (PSF113_RS57875), protein RlpB (PSF113_RS56535), protein FecA (PSF113_RS55030), porin (PSF113_RS39290), porin (PSF113_RS36845), murein transglycosylase (PSF113_RS54350), membrane protein (PSF113_RS55445), membrane protein (PSF113_RS54915), membrane protein (PSF113_RS40895), maltoporin (PSF113_RS35615), and LPS-assembly protein LptD (PSF113_RS57585).

In another embodiment, the identified genetic element in the method is an enzyme for naphthenic degradation in Pseudomonas selected from the group consisting of: short-chain oxidoreductase (PP_2789), putative oxidoreductase (PP_0256), paraquat-inducible protein A (PP_0598), p-nitrobenzoate reductase NfnB (PP_3657), oxidoreductase (PP_4020), paraquat-inducible protein A (PP_5745), coniferyl-aldehyde dehydrogenase (calB), dihydroflavonol-4-reductase (PP_2986), short-chain oxidoreductase (PP_1817), dehydrogenase (PP_1661), FMN-dependent NADH-azoreductase (azoR2), 2-carboxybenzaldehyde reductase (yajO), ring-cleaving dioxygenase (PP_3328), 2-alkenal reductase (PSF113_4895), aldo-keto reductase (PSF113_4805), dehydrogenase (PSF113_0191), dehydrogenase (PSF113_4571), FAD-binding oxidoreductase (PSF113_3070), FAD-linked oxidoreductase (PSF113_3227), FMN-dependent NADH-azoreductase (PSF113_1483), Flavin-dependent oxidoreductase (PSF113_0134), dehydrogenase (PSF113_2405), dehydrogenase (PSF113_4207), NAD(P)-dependent oxidoreductase (PSF113_5330), acyl-CoA thioesterase (PSF113_5500), and OHCU decarboxylase (PSF113_4335).

In another embodiment, the identified genetic element in the method is a pathway related to naphthenic acid (NA) degradation in Pseudomonas selected from the group consisting of: protocatechuate degradation II, CFA biosynthesis, syringate degradation, benzoate degradation I, androstenedione degradation, phenylacetate degradation I, L-phenylalanine biosynthesis I, and nicotinate degradation.

In another aspect, the present invention is directed to providing a gene expression profile being predictive for the specific response of a gene, enzyme or pathway comprising determining gene expression profiles from at least two microorganisms involved in the degradation activity of toxic organic compounds in the microorganisms.

In an embodiment, the method comprises the steps of:

(i) determining gene expression profiles from a first microorganism isolated from a contaminated environment in the presence of extracted naphthenic acid (NA);

(ii) determining gene expression profiles form a second microorganism isolated from a contaminated environment in the absence of extracted naphthenic acid (NA); and

(iii) identifying genes that are expressed at different levels in the contaminated environment in the presence versus in the absence of the extracted naphthenic acid (NA) as the gene expression profile that predicts response to the degradation activity of toxic organic compounds.

Preferably, the gene expression profiles of the method are based on the RNA expression levels of the at least two microorganism strains, preferably Pseudomonas putida and Pseudomonas fluorescens.

### m) Extraction Methods

In one aspect, the present invention is directed to a method of extracting organic compounds for selectively concentrating nitrogen-containing species from oil sands affected waters (OSPW), comprising: a) removing particulate matter from OSPW and acidifying the OSPW; b) liquid extracting the OSPW with an organic solvent one or more times; and c) evaporating the organic solvent and dissolving remaining organic matter in a solution containing a solvent and water.

In one embodiment, the particulate matter is removed using vacuum filtration through glass fibre filters.

In another embodiment, the pH of the OSPW is lowered to pH 2.

In another embodiment, the organic solvent is dichloromethane (DCM).

In another embodiment, the solution is a 50:50 solution of acetonitrile and water at 1× concentration of an original sample of the OSPW.

### n) Polynucleotides

In accordance with the foregoing, the present disclosure relates to the discovery that certain bacterial enzymes are intimately involved with the naphthenic acid (NA) degradation activity required for biodegradation of toxic organic compounds found in OPSW. As such, the inventors surprisingly discovered that these enzymes are important targets for modifications to increase their naphthenic acid degradation activity so as to be useful in bioremediation of contaminated waste.

Specifically, in one aspect, the present disclosure relates to a polynucleotide encoding an enzyme having naphthenic acid (NA) degradation activity, preferably increased NA degradation activity. Preferably, the polynucleotide comprises an modified nucleic acid that encodes an enzyme having an increased naphthenic acid degradation activity when compared to a control enzyme that lacks the genetic modification. The modification may result in increases in the expression level and/or increases in the activity of the enzyme. By “increased” it is meant that the naphthenic acid degradation activity for the modified enzyme may improve by at least 0.5×, 1× or 2× as compared to the control enzyme with the wild-type enzyme.

It will be understood that the increased in naphthenic acid degradation activity can be determined by any methods known to those skilled in the art. It is preferred that the increased in naphthenic acid (NA) degradation activity is evidenced by a higher level of degraded toxic organic compounds (e.g., degraded naphthenic acid (NA)).

In an embodiment, the polynucleotide can be classified as polynucleotide that is associated with or is an outer membrane associated polynucleotide, a transporter polynucleotide, an efflux pump polynucleotide, or a reactive oxygen species removal polynucleotide.

In another embodiment, the enzyme encoded by the polynucleotide is selected from the group consisting of: catalase (katA), alkyl hydroperoxide reductase subunit F (ahpF), thioredoxin reductase (trxB), peroxiredoxin (tsaA), hydroperoxy fatty acid reductase Gpx1 (gpx), multidrug transporter membrane protein (mdtB), fusaric acid resistance protein (PP_1266), RND transporter membrane fusion protein (PP_3301), multidrug transporter membrane protein (mdtC), multidrug RND transporter MexF (mexF), RND family transporter (PP_5173), Bcr/CflA family multidrug resistance transporter (PP_3304), RND family transporter (PP_3302), uronate transporter (PP_2837), glucarate transporter (gudP), transporter (PP_3250), tartrate MFS transporter (PP_3391), MFS transporter (PP_3566), metabolite transport protein YhjE (yhjE), carbohydrate efflux transporter (yhhS), aromatic compound MFS transporter (PP_3658), acyltransferase (PP_1700), TonB-dependent receptor (PP_3340), porin F (oprF), peptidoglycan-associated lipoprotein (oprL), phenylacetic acid-specific porin (phaK), outer-membrane porin E (oprE), outer-membrane porin D (oprQ), outer membrane protein assembly factor (bamA-II), outer membrane protein assembly factor (bamA-I), outer membrane ferric citrate porin (fecA), OmpA family protein (PP_4198), LPS-assembly protein LptD (IptD), lipid A 3-O-deacylase (pagL-I), glycine-glutamate dipeptide porin (opdP), hypothetical protein (PP_4115), ferrioxamine receptor (PP_0160), ferrichrome-iron receptor (PP_4755), ferric siderophore receptor (PP_3330), ferric siderophore receptor (PP_3325), aromatic compound-specific porin (PP_3656), ferric siderophore receptor (PP_0535), alginate production protein AlgE (algE), alginate biosynthesis protein AlgK (algK), thioredoxin (PSF113_RS59500), thioredoxin (PSF113_RS59220), thioredoxin reductase (PSF113_RS41395), stringent starvation protein A (PSF113_RS54790), peroxidase (PSF113_RS53615), peroxiredoxin (PSF113_RS39210), glutathione peroxidase (PSF113_RS55240), glutathione peroxidase (PSF113_RS39005), catalase (PSF113_RS52605), catalase (PSF113_RS57160), alkyl hydroperoxide reductase subunit F (PSF113_RS42115), secretion protein HylD (PSF113_RS36900), RND transporter MFP subunit (PSF113_RS54645), RND efflux transporter (PSF113_RS35960), multidrug transporter MatE (PSF113_RS50665), multidrug efflux RND transporter permease subunit (PSF113_RS44170), multidrug ABC transporter substrate-binding protein (PSF113_RS50525), acriflavine resistance protein B (PSF113_RS43745), MFS transporter (PSF113_RS53065), MFS transporter (PSF113_RS50550), 4-hydroxybenzoate transporter (PSF113_RS37090), MFS transporter (PSF113_RS38620), TonB-dependent receptor (PSF113_RS57875), protein RlpB (PSF113_RS56535), protein FecA (PSF113_RS55030), porin (PSF113_RS39290), porin (PSF113_RS36845), murein transglycosylase (PSF113_RS54350), membrane protein (PSF113_RS55445), membrane protein (PSF113_RS54915), membrane protein (PSF113_RS40895), maltoporin (PSF113_RS35615), and LPS-assembly protein LptD (PSF113_RS57585).

In another embodiment, the enzyme encoded by the polynucleotide is selected from the group consisting of: short-chain oxidoreductase (PP_2789), putative oxidoreductase (PP_0256), paraquat-inducible protein A (PP_0598), p-nitrobenzoate reductase NfnB (PP_3657), oxidoreductase (PP_4020), paraquat-inducible protein A (PP_5745), coniferyl-aldehyde dehydrogenase (calB), dihydroflavonol-4-reductase (PP_2986), short-chain oxidoreductase (PP_1817), dehydrogenase (PP_1661), FMN-dependent NADH-azoreductase (azoR2), 2-carboxybenzaldehyde reductase (yajO), ring-cleaving dioxygenase (PP_3328), 2-alkenal reductase (PSF113_4895), aldo-keto reductase (PSF113_4805), dehydrogenase (PSF113_0191), dehydrogenase (PSF113_4571), FAD-binding oxidoreductase (PSF113_3070), FAD-linked oxidoreductase (PSF113_3227), FMN-dependent NADH-azoreductase (PSF113_1483), Flavin-dependent oxidoreductase (PSF113_0134), dehydrogenase (PSF113_2405), dehydrogenase (PSF113_4207), NAD(P)-dependent oxidoreductase (PSF113_5330), acyl-CoA thioesterase (PSF113_5500), and OHCU decarboxylase (PSF113_4335).

### o) Vectors

The present disclosure also teaches vector expression system which comprises a polynucleotide or polynucleotides of the present disclosure. Host cells which are engineered with vectors of the disclosure and the production of polynucleotides of the disclosure by recombinant techniques are also encompassed by the disclosure.

In accordance with this aspect of the invention, the vector may be, for example, a plasmid vector, a single or double-stranded phage vector, or a single or double-stranded RNA or DNA viral vector. In certain embodiments in this regard, the vectors provide for specific expression or mediate chromosomal integration for expression. Such specific expression may be inducible expression or expression only in certain types of cells or both inducible and cell-specific. Particular among inducible vectors are vectors that can be induced for expression by environmental factors that are easy to manipulate, such as temperature and nutrient additives. A variety of vectors suitable to this aspect of the invention, including constitutive and inducible expression vectors for use in prokaryotic and eukaryotic hosts, are well known and employed routinely by those of skill in the art. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. All of these may be used for expression in accordance with this aspect of the present disclosure.

### p) Host Cells

As hereinbefore mentioned, the present disclosure also teaches host cells which are engineered with vectors as described herein. The methods disclosed herein may be used to optimize chassis strains to be used in a variety of biological applications according to the present invention. For example, the methods disclosed herein can be used to create chassis strains de novo from strains with desirable traits (e.g., improved NA degradation activity), producing a new chassis strain with desired characteristics. Methods of making synthetic cells are also provided.

Polynucleotide construct in host cells can be used in a conventional manner to produce the gene product encoded by the polynucleotide. The subject polynucleotides or polypeptides products or isoforms or parts thereof, may be obtained by expression in a suitable host cell using techniques known in the art. Suitable host cells include prokaryotic organisms or cell lines, for example bacterial cells. Methods for transforming or transfecting cells to express foreign DNA are well known in the art (See for example, Itakura et al., U.S. Pat. No. 4,704,362; Hinnen et al., 1978; Murray et al., U.S. Pat. No. 4,801,542; McKnight et al., U.S. Pat. No. 4,935,349; Hagen et al., U.S. Pat. No. 4,784,950; Axel et al., U.S. Pat. No. 4,399,216; Goeddal et al., U.S. Pat. No. 4,766,075 and Sambrook et al., 1989, Molecular Cloning, 2nd Edition, Cold Spring Harbor Laboratory Press, Cold Spring Harbour, N.Y. all of which are incorporated herein by reference). Representative examples of appropriate hosts include bacterial cells, such as, Pseudomonas putida or Pseudomonas fluorescens.

Host cells can be engineered to incorporate polynucleotides and express polynucleotides of the present disclosure. Introduction of polynucleotides into the host cell can be effected by calcium phosphate transfection, DEAR-dextran mediated transfection, transvection, microinjection, cationic lipid-mediated transfection, electroporation, transduction, scrape loading, ballistic introduction, infection or other methods known to those skilled in the art. Such methods are described, for example, in many standard laboratory manuals, such as Davis et al., 1986, Basic Methods in Molecular Biology, Elsevier, N.Y. and Sambrook et al., 1989, Molecular Cloning, 2^{nd} Edition, Cold Spring Harbor Laboratory Press, Cold Spring Harbour, N.Y.

### q) Engineered Microorganisms

In another aspect, the present disclosure relates to an engineered microorganism comprising one or more of the polynucleotides as described herein. In certain embodiments, the modification increases the naphthenic acid degradation activity of the engineered microorganisms.

As used herein, the expression “engineered” refers to one or more alterations of a nucleic acid, e.g., the nucleic acid within an organism's genome. For example, engineered can refer to genetic alterations, additions, and/or deletion of genes. A genetically engineered microorganism can also refer to a microorganism with an added, deleted and/or altered gene. For example, an engineered microorganism can be from a genetically modified bacterium. It is preferred that the engineered microorganism is Pseudomonas putida or Pseudomonas fluorescens.

In another embodiment, the engineered microorganism as described herein degrades toxic organic compounds, as contained in contaminated environments. Preferably, the engineered microorganism has enhanced degradation of toxic organic compounds as compared to a control microorganism. It will be understood that toxic compounds may include any toxic organic compounds commonly present in OPSW. Preferably, the toxic compounds according to the present disclosure are selected from the group consisting of naphthenic acid (NA), polycyclic aromatic hydrocarbons (PAH), benzene, toluene, ethyl benzene, xylenes, phenols, heavy metals, ions and a combination thereof.

More preferably, the toxic compounds according to the present disclosure are toxic organic compounds having a formula:

C_{c}H_{2c+Z}O₂,

wherein each c is independently an integer from 5 to 25 and Z is zero or an even integer representing hydrogen deficiency due to rings or double bonds. It is especially preferable that the toxic compounds of the formula above, wherein the double bonds are a double bond equivalent (DBE) between 1 and 10. It is particularly preferred that the toxic compounds are C_{c}H_{2c+Z}NₙOₒSₛ.

### r) Gene Expression Profiling

In another aspect, the present disclosure relates to a method of identifying genes, enzymes or pathways involved in degradation activity of toxic organic compounds in microorganisms. The method comprises

generating gene expression profiles for microorganisms isolated from water affected with toxic organic compounds,

identifying genes, enzymes, or pathways involved in adaptation of microorganisms to the compounds, and

identifying genes, enzymes, or pathways involved in degradation of the compounds by the microorganisms.

In another aspect, the present disclosure also relates to a method of RNA expression analysis for identifying genes, enzymes, or pathways involved in degradation activity of toxic organic compounds in microorganisms. The method comprises:

calculating a Reaction Perturbation Score (RPS) and a Pathway Perturbation Score (PPS) to determine upregulation of an enzyme associated with a pathway for degradation of toxic organic compounds based on RNA expression levels in response to the degradation of the toxic organic compounds, and

identifying pathway enzymes, pathway inputs and terminal catabolites of the pathway.

In certain embodiments of the method, the toxic organic compounds are selected from the group consisting of naphthenic acid (NA), polycyclic aromatic hydrocarbons (PAH), benzene, toluene, ethyl benzene, xylenes, phenols, heavy metals, ions and a combination thereof, preferably naphthenic acid (NA).

In other embodiments of the method, the microorganism is Pseudomonas putida or Pseudomonas fluorescens.

### s) Compositions

In another aspect, the present disclosure also relates to a composition for biodegradation of toxic organic compounds. The composition comprises one or more of the engineered microorganisms as disclosed herein, and an acceptable carrier effective for delivery of the engineered microorganisms to a contaminated environment. Preferably, the composition further comprises a photocatalyst.

### t) Biodegradation Method

In another aspect, the present disclosure relates to a method for biodegradation of toxic organic compounds which are present in a contaminated environment. The method comprises:

contacting the contaminated environment with a microbial consortium comprising at least one or more of the engineered microorganisms as described herein; and

maintaining the microbial consortium in contact with the contaminated environment for a time that is effective for the microbial consortium to biodegrade the toxic compounds.

It is also preferred that the method according to the disclosure reduces the concentration of toxic organic compounds in the contaminated environment.

In all embodiments of the present disclosure, all percentages, parts and ratios are based upon the total weight of the compositions of the present disclosure, unless otherwise specified. All such weights as they pertain to listed ingredients are based on the active level and, therefore do not include solvents or by-products that may be included in commercially available materials, unless otherwise specified.

All ratios are weight ratios unless specifically stated otherwise. All temperatures are in Celsius degrees (° C.), unless specifically stated otherwise. All dimensions and values disclosed herein (e.g., quantities, percentages, portions, and proportions) are not to be understood as being strictly limited to the exact numerical values recited. Instead, unless otherwise specified, each such dimension or value is intended to mean both the recited value and a functionally equivalent range surrounding that value. For example, a dimension disclosed as “40 mm” is intended to mean “about 40 mm.”

While specific embodiments have been described and illustrated, such embodiments should be considered illustrative of the subject matter described herein and not as limiting the claims as construed in accordance with the relevant jurisprudence.

### REFERENCES AND NOTES

1. McQueen, A. D., Kinley, C. M., Hendrikse, M., Gaspari, D. P., Calomeni, A. J., Iwinski, K. J., Castle, J. W., Haakensen, M. C., Peru, K. M., Headley, J. V., et al. (2017). A risk-based approach for identifying constituents of concern in oil sands process-affected water from the Athabasca Oil Sands region. Chemosphere 173, 340-350.
2. Morandi, G. D., Wiseman, S. B., Pereira, A., Mankidy, R., Gault, I. G. M., Martin, J. W., and Giesy, J. P. (2015). Effects-Directed Analysis of Dissolved Organic Compounds in Oil Sands Process-Affected Water. Environ. Sci. Technol. 49, 12395-12404.
3. Brown, L. D., and Ulrich, A. C. (2015). Oil sands naphthenic acids: A review of properties, measurement, and treatment. Chemosphere 127, 276-290.
4. Hughes, S. A., Mahaffey, A., Shore, B., Baker, J., Kilgour, B., Brown, C., Peru, K. M., Headley, J. V., and Bailey, H. C. (2017). Using ultrahigh-resolution mass spectrometry and toxicity identification techniques to characterize the toxicity of oil sands process-affected water: The case for classical naphthenic acids. Environ. Toxicol. Chem. 36, 3148-3157.
5. Marentette, J. R., Frank, R. A., Hewitt, L. M., Gillis, P. L., Bartlett, A. J., Brunswick, P., Shang, D., and Parrott, J. L. (2015). Sensitivity of walleye (Sander vitreus) and fathead minnow (Pimephales promelas) early-life stages to naphthenic acid fraction components extracted from fresh oil sands process-affected waters. Environ. Pollut. 207, 59-67.
6. Sands, O. I. L. (2013). Tailings.
7. Alberta Energy Regulator (2017). Directive 085: Fluid Tailings Management for Oil Sands Mining Projects.
8. Alberta, G. of (2015). Lower Athabasca Region—Tailings Management Framework for the Mineable Athabasca Oil Sands.
9. Martin, J. W. (2015). The Challenge: Safe release and reintegration of oil sands process-affected water. Environ. Toxicol. Chem. 34, 2682.
10. Li, C., Fu, L., Stafford, J., Belosevic, M., and Gamal EI-Din, M. (2017). The toxicity of oil sands process-affected water (OSPW): A critical review. Sci. Total Environ. 601-602, 1785-1802.
11. Morandi, G. D., Wiseman, S. B., Guan, M., Zhang, X. W., Martin, J. W., and Giesy, J. P. (2017). Elucidating mechanisms of toxic action of dissolved organic chemicals in oil sands process-affected water (OSPW). Chemosphere 186, 893-900.
12. Barrow, M. P., Witt, M., Headley, J. V, Peru, K. M., Selman, M. H. J., McDonnell, L. a, Palmblad, M., Ruhaak, L. R., Deelder, A. M., and Wuhrer, M. (2010). Athabasca oil sands process water: characterization by atmospheric pressure photoionization and electrospray ionization fourier transform ion cyclotron resonance mass spectrometry. Anal. Chem. 82, 3727-3735.
13. Grewer, D. M., Young, R. F., Whittal, R. M., and Fedorak, P. M. (2010). Naphthenic acids and other acid-extractables in water samples from Alberta: What is being measured? Sci. Total Environ. 408, 5997-6010.
14. Clemente, J. S., and Fedorak, P. M. (2005). A review of the occurrence, analyses, toxicity, and biodegradation of naphthenic acids. Chemosphere 60, 585-600.
15. Kannel, P. R., and Gan, T. Y. (2012). Naphthenic acids degradation and toxicity mitigation in tailings wastewater systems and aquatic environments: A review. J. Environ. Sci. Heal.—Part A Toxic/Hazardous Subst. Environ. Eng. 47, 1-21.
16. Sun, C., Shotyk, W., Cuss, C. W., Donner, M. W., Fennell, J., Javed, M., Noernberg, T., Poesch, M., Pelletier, R., Sinnatamby, N., et al. (2017). Characterization of Naphthenic Acids and Other Dissolved Organics in Natural Water from the Athabasca Oil Sands Region, Canada. Environ. Sci. Technol. 51, 9524-9532.
17. Pereira, A. D. S., Bhattacharjee, S., and Martin, J. W. (2013). Characterization of Oil Sands Process Affected Waters by Liquid Chromatography Orbitrap Mass Spectrometry. Environ. Sci. Technol. 47, 5504-5513.
18. Headley, J. V., Peru, K. M., Mohamed, M. H., Frank, R. A., Martin, J. W., Hazewinkel, R. R. O., Humphries, D., Gurprasad, N. P., Hewitt, L. M., Muir, D. C. G., et al. (2013). Chemical fingerprinting of naphthenic acids and oil sands process waters-A review of analytical methods for environmental samples. J. Environ. Sci. Heal.—Part A Toxic/Hazardous Subst. Environ. Eng. 48, 1145-1163.
19. Quinlan, P. J., and Tam, K. C. (2015). Water treatment technologies for the remediation of naphthenic acids in oil sands process-affected water. Chem. Eng. J. 279, 696-714.
20. Quagraine, E. K., Headley, J. V., and Peterson, H. G. (2005). Is biodegradation of bitumen a source of recalcitrant naphthenic acid mixtures in oil sands tailing pond waters? J. Environ. Sci. Heal.—Part A Toxic/Hazardous Subst. Environ. Eng. 40, 671-684.
21. Han, X., Scott, A. C., Fedorak, P. M., Bataineh, M., and Martin, J. W. (2008). Influence of molecular structure on the biodegradability of naphthenic acids. Environ. Sci. Technol. 42, 1290-1295.
22. de Oliveira Livera, D., Leshuk, T., Peru, K. M., Headley, J. V., and Gu, F. (2018). Structure-reactivity relationship of naphthenic acids in the photocatalytic degradation process. Chemosphere 200, 180-190.
23. COSIA's Water Challenges COSIA Challenge #0014: Passive Organics Treatment Technology. 2015.
24. Herman, D. C., Fedorak, P. M., MacKinnon, M. D., and Costerton, J. W. (1994). Biodegradation of naphthenic acids by microbial populations indigenous to oil sands tailings. Can. J. Microbiol. 40, 467-477.
25. Scott, A. C., MacKinnon, M. D., and Fedorak, P. M. (2005). Naphthenic acids in athabasca oil sands tailings waters are less biodegradable than commercial naphthenic acids. Environ. Sci. Technol. 39, 8388-8394.
26. Vaiopoulou, E., Misiti, T. M., and Pavlostathis, S. G. (2015). Removal and toxicity reduction of naphthenic acids by ozonation and combined ozonation-aerobic biodegradation. Bioresour. Technol. 179, 339-347.
27. Misiti, T. M., Tezel, U., and Pavlostathis, S. G. (2014). Effect of alkyl side chain location and cyclicity on the aerobic biotransformation of naphthenic acids. Environ. Sci. Technol. 48, 7909-7917.
28. Paslawski, J., Nemati, M., Hill, G., and Headley, J. (2009). Biodegradation kinetics of trans-4-methyl-1-cyclohexane carboxylic acid in continuously stirred tank and immobilized cell bioreactors. J. Chem. Technol. Biotechnol. 84, 992-1000.
29. Gunawan, Y., Nemati, M., and Dalai, A. (2014). Biodegradation of a surrogate naphthenic acid under denitrifying conditions. Water Res. 51, 11-24.
30. S. T. Kellogg, D. K. C. and A. M. C. (1981). Plasmid-Assisted Molecular Breeding: New Technique for Enhanced Biodegradation of Persistent Toxic Chemicals. Science (80-.). 214, 1133-1135.
31. Dvořák, P., Nikel, P. I., Damborský, J., and de Lorenzo, V. (2017). Bioremediation 3.0: Engineering pollutant-removing bacteria in the times of systemic biology. Biotechnol. Adv. 35, 845-866.
33. Morandi, G. D., Wiseman, S., Pereira, A. D. S., Mankidy, R., Gault, I. G. M., Martin, J. W., and Giesy, J. P. (2015). Effects-Directed Analysis of Dissolved Organic Compounds in Oil Sands Process Affected Water. Environ. Sci. Technol. 49, 12395-12404.
34. de Oliveira Livera, D., Leshuk, T., Peru, K. M., Headley, J. V., and Gu, F. (2018). Structure-reactivity relationship of naphthenic acids in the photocatalytic degradation process. Chemosphere 200, 180-190.
35. Xiao, J., Guo, L., Wang, S., and Lu, Y. (2010). Comparative impact of cadmium on two phenanthrene-degrading bacteria isolated from cadmium and phenanthrene co-contaminated soil in China. J. Hazard. Mater. 174, 818-823.
36. Yue, S., Ramsay, B. A., and Ramsay, J. A. (2015). Biodegradation of naphthenic acid surrogates by axenic cultures. Biodegradation 26, 313-325.
37. Phillips, L. A., Armstrong, S. A., Headley, J. V., Greer, C. W., and Germida, J. J. (2010). Shifts in root-associated microbial communities of typha latifolia growing in naphthenic acids and relationship to plant health. Int. J. Phytoremediation 12, 745-760.
38. Del Rio, L. F., Hadwin, A. K. M., Pinto, L. J., MacKinnon, M. D., and Moore, M. M. (2006). Degradation of naphthenic acids by sediment micro-organisms. J. Appl. Microbiol. 101, 1049-1061.
39. Johnson, R. J., Smith, B. E., Sutton, P. A., McGenity, T. J., Rowland, S. J., and Whitby, C. (2011). Microbial biodegradation of aromatic alkanoic naphthenic acids is affected by the degree of alkyl side chain branching. ISME J. 5, 486-496.
41. EPA (2012). Sustainable Futures/P2 Framework Manual 2012 EPA-748-B12-001 5. Estimating Physical/Chemical and Environmental Fate Properties with EPI Suite™. Sustain. Futur./P2 Framew. Man. 2012 EPA-748-B12-001, 1-22.
42. [Springer Handbooks] Chang Samuel Hsu ( [symbol] ), Paul R. Robinson (eds.)—Springer Handbook of Petroleum Technology (2017, Springer International Publishing).
43. Hancock, R. E. W., and Brinkman, F. S. L. (2002). Function of Pseudomonas Porins in Uptake and Efflux. Annu. Rev. Microbiol. 56, 17-38.
44. Pao, S. S., Paulsen, I. A. N. T., and Saier, M. H. (1998). Major Facilitator Superfamily-1, 62, 1-34.
45. Wiseman, S. B., He, Y., Din, M. G., Martin, J. W., Jones, P. D., Hecker, M., and Giesy, J. P. (2013). Comparative Biochemistry and Physiology, Part C Transcriptional responses of male fathead minnows exposed to oil sands process-affected water. Comp. Biochem. Physiol. Part C 157, 227-235.
46. Hu, K. H., Liu, E., Dean, K., Gingras, M., DeGraff, W., and Trun, N. J. (1996). Overproduction of three genes leads to camphor resistance and chromosome condensation in Escherichia coli. Genetics 143, 1521-1532.
47. BY Sergey A. Selifonov Center for Environmental Diagnostics and Bioremediation, University of West Florida, Building 58, 11000 University Parkway, (1992). 186, 1429-1436.
48. Ramos, J. L., Cuenca, M. S., Molina-Santiago, C., Segura, A., Duque, E., \acute{G}omez-Garciá, M. R., Udaondo, Z., and Roca, A. (2015). Mechanisms of solvent resistance mediated by interplay of cellular factors in Pseudomonas putida. FEMS Microbiol. Rev. 39, 555-566.
49. Putman, M., van Veen, H. W., and Konings, W. N. (2000). Molecular Properties of Bacterial Multidrug Transporters. Microbiol. Mol. Biol. Rev. 64, 672-693.
50. Crutcher, F. K., Puckhaber, L. S., Stipanovic, R. D., Bell, A. A., Nichols, R. L., Lawrence, K. S., and Liu, J. (2017). Microbial Resistance Mechanisms to the Antibiotic and Phytotoxin Fusaric Acid. J. Chem. Ecol. 43, 996-1006.
51. Zgurskaya, H. I., Krishnamoorthy, G., Ntreh, A., and Lu, S. (2011). Mechanism and function of the outer membrane channel ToIC in multidrug resistance and physiology of enterobacteria. Front. Microbiol. 2, 1-13.
52. Bentley, J., Hyatt, L. S., Ainley, K., Parish, J. H., Herbert, R. B., and White, G. R. (1993). Cloning and sequence analysis of an Escherichia coli gene conferring bicyclomycin resistance. Gene 127, 117-120.
53. Koronakis, V., Sharff, A., Koronakis, E., Luisi, B., and Hughes, C. (2000). Crystal structure of the bacterial membrane protein ToIC central to multidrug efflux and protein export. Nature 405, 914-919.
54. Mima, T., Joshi, S., Gomez-Escalada, M., and Schweizer, H. P. (2007). Identification and characterization of TriABC-OpmH, a triclosan efflux pump of Pseudomonas aeruginosa requiring two membrane fusion proteins. J. Bacteriol. 189, 7600-7609.
55. Babin, B. M., Atangcho, L., van Eldijk, M. B., Sweredoski, M. J., Moradian, A., Hess, S., Tolker-Nielsen, T., Newman, D. K., Tirrell, D. a., Cellular, A., et al. (2017). crossm Selective Proteomic Analysis of. M Bio 8, 1-16.
56. Yee, R., Cui, P., Shi, W., Feng, J., and Zhang, Y. (2015). Genetic Screen Reveals the Role of Purine Metabolism in Staphylococcus aureus Persistence to Rifampicin. Antibiotics 4, 627-642.
57. Rallu, F., Gruss, A., Ehrlich, S. D., and Maguin, E. (2000). Acid- and multistress-resistant mutants of Lactococcus lactis: Identification of intracellular stress signals. Mol. Microbiol. 35, 517-528.
58. Rodionov, D. A., Li, X., Rodionova, I. A., Yang, C., Sorci, L., Dervyn, E., Martynowski, D., Zhang, H., Gelfand, M. S., and Osterman, A. L. (2008). Transcriptional regulation of NAD metabolism in bacteria: Genomic reconstruction of NiaR (YrxA) regulon. Nucleic Acids Res. 36, 2032-2046.
59. Johnson, M. D. L., Echlin, H., Dao, T. H., and Rosch, J. W. (2015). Characterization of NAD salvage pathways and their role in virulence in Streptococcus pneumoniae. Microbiol. (United Kingdom) 161, 2127-2136.
60. Noda, S., Shirai, T., Mori, Y., Oyama, S., and Kondo, A. (2017). Engineering a synthetic pathway for maleate in Escherichia coli. Nat. Commun. 8, 1-7.
63. Okubo, Y., Yang, S., Chistoserdova, L., and Lidstrom, M. E. (2010). Alternative route for glyoxylate consumption during growth on two-carbon compounds by Methylobacterium extorquens AM1. J. Bacteriol. 192, 1813-1823.
64. Koide, Y., Honma, M., and Shimomura, T. (1977). <scp>|</scp>-Alanine-α-Keto Acid Aminotransferase of Pseudomonas sp. Agric. Biol. Chem. 41, 781-784.
65. Blakley, E. R. (1974). The microbial degradation of cyclohexanecarboxylic acid: a pathway involving aromatization to form p-hydroxybenzoic acid. J. Microbiol. 20, 1297-1306.
66. Wang, X., Chen, M., Xiao, J., Hao, L., Crowley, D. E., Zhang, Z., Yu, J., Huang, N., Huo, M., and Wu, J. (2015). Genome sequence analysis of the naphthenic acid degrading and metal resistant bacterium cupriavidus gilardii CR3. PLoS One 10, 1-21.
67. Brzostowicz, P. C., Blasko, M. S., and Rouviere, P. E. (2002). Identification of two gene clusters involved in cyclohexanone oxidation in Brevibacterium epidermidis strain HCU. Appl. Microbiol. Biotechnol. 58, 781-789.
68. Stirling, D. ian, and Dalton, H. (1979). The fortuitous oxidation and cometabolism of various carbon compounds by whole-cell suspensions of Methylococcus capsulatus (Bath). FEMS Microbiol. Lett. 5, 315-318.
69. Karp, P. D., Latendresse, M., Paley, S. M., Krummenacker, M., Ong, Q. D., Billington, R., Kothari, A., Weaver, D., Lee, T., Subhraveti, P., et al. (2016). Pathway tools version 19.0 update: Software for pathway/genome informatics and systems biology. Brief. Bioinform. 17, 877-890.
70. Nakanishi, M., Yatome, C., Ishida, N., and Kitade, Y. (2001). Putative ACP Phosphodiesterase Gene (acpD) Encodes an Azoreductase. J. Biol. Chem. 276, 46394-46399.
71. Pieper, D. H. (2005). Aerobic degradation of polychlorinated biphenyls. Appl. Microbiol. Biotechnol. 67, 170-191.
72. Hein, S., Tran, H., and SteinbUchel, A. (1998). Synechocystis sp. PCC6803 possesses a two-component polyhydroxyalkanoic acid synthase similar to that of anoxygenic purple sulfur bacteria. Arch. Microbiol. 170, 162-170.
73. Bolger, A. M., Lohse, M., and Usadel, B. (2014). Trimmomatic: A flexible trimmer for Illumina sequence data. Bioinformatics 30, 2114-2120.
74. Crusoe, M. R., Alameldin, H. F., Awad, S., Boucher, E., Caldwell, A., Cartwright, R., Charbonneau, A., Constantinides, B., Edvenson, G., Fay, S., et al. (2015). The khmer software package: enabling efficient nucleotide sequence analysis. F1000Research, 1-10.
75. Bankevich, A., Nurk, S., Antipov, D., Gurevich, A. A., Dvorkin, M., Kulikov, A. S., Lesin, V. M., Nikolenko, S. I., Pham, S., Prjibelski, A. D., et al. (2012). SPAdes: A New Genome Assembly Algorithm and Its Applications to Single-Cell Sequencing. J. Comput. Biol. 19, 455-477.
76. Konwar, K. M., Hanson, N. W., Page, A. P., and Hallam, S. J. (2013). MetaPathways: A modular pipeline for constructing pathway/genome databases from environmental sequence information. BMC Bioinformatics 14.
77. Huang, D. W., Sherman, B. T., and Lempicki, R. A. (2009). Bioinformatics enrichment tools: Paths toward the comprehensive functional analysis of large gene lists. Nucleic Acids Res. 37, 1-13.

What is claimed is:

1. A method of identifying genetic elements involved in biodegradation of environmental compounds by microorganisms, comprising:
identifying a microorganism strain capable of assimilating a toxic organic compound isolated from an affected environment as a carbon source by exposing the microorganism strain to an isolated toxic organic compound, wherein the isolated toxic organic compound has a chemical characteristic;
preparing a gene expression profile of the microorganism strain grown i) in the presence of the toxic organic compound and ii) in the absence of the toxic organic compound, to determine an RNA expression level of one or more differentially expressed genes (DEG; and
identifying a genetic element involved in the response of the microorganism strain to exposure to the toxic organic compound, by combining the chemical characteristic of the toxic organic compound with the gene expression profile of the microorganism strain.

2-46. (canceled)

47. The method of claim 1, wherein the affected environment is oil sands process-affected waters (OSPW).

48. The method of claim 47, further comprising prior to identifying the microorganism strain, isolating the toxic organic compound from a sample of the affected environment, and
determining the chemical characteristic of the toxic organic compound.

49. The method of claim 48, wherein the isolated toxic organic compound is a naphthenic acids fraction compound (NAFC) or a naphthenic acid (NA).

50. The method of claim 48, wherein the chemical characteristic of the toxic organic compound is a molecular formula, a retention time, a double bond equivalent (DBE), or a carbon number.

51. The method of claim 47, wherein identifying the microorganism strain comprises:
selectively enriching a mixed population of naturally-occurring microorganism strains in OSPW in growth media containing successively increasing concentrations of the toxic organic compound, and
sequencing polynucleotides from an isolated single colony from the enriched mixed population of microorganism strains to identify the microorganism strain of the isolated single colony.

52. The method of claim 49, wherein identifying the genetic element comprises identifying genetic elements related to adaptation or physiological response of the microorganism strain to exposure to NAFC or NA, wherein the method comprises:
reconstructing one or more general metabolic pathways using the gene expression profile, and
enriching the reconstructed metabolic pathway and assigning the reconstructed metabolic pathway an enrichment score.

53. The method of claim 52, wherein the identified genetic element is: an amino acids biosynthesis/degradation pathway, a purine metabolism pathway, a pyrimidine metabolism pathway, nicotinate and nicotinamide metabolism pathways, a fatty acids degradation pathway, a propanoate metabolism pathway, a 2-oxocarboxylic acid metabolism pathway, a lipopolysaccharide biosynthesis pathway, or a butanoate metabolism pathway.

54. The method of claim 52, further comprising analyzing internal transport, activation and initiation of degradation in the microorganism strain through functional gene clustering of the gene expression profile.

55. The method of claim 54, wherein the identified genetic element induced upon exposure of the microorganism strain to NAFC or NA is: catalase (katA), alkyl hydroperoxide reductase subunit F (ahpF), thioredoxin reductase (trxB), peroxiredoxin (tsaA), hydroperoxy fatty acid reductase Gpx1 (gpx), multidrug transporter membrane protein (mdtB), fusaric acid resistance protein (PP_1266), RND transporter membrane fusion protein (PP_3301), multidrug transporter membrane protein (mdtC), multidrug RND transporter MexF (mexF), RND family transporter (PP_5173), Bcr/CflA family multidrug resistance transporter (PP_3304), RND family transporter (PP_3302), uronate transporter (PP_2837), glucarate transporter (gudP), transporter (PP_3250), tartrate MFS transporter (PP_3391), MFS transporter (PP_3566), metabolite transport protein YhjE (yhjE), carbohydrate efflux transporter (yhhS), aromatic compound MFS transporter (PP_3658), acyltransferase (PP_1700), TonB-dependent receptor (PP_3340), porin F (oprF), peptidoglycan-associated lipoprotein (oprL), phenylacetic acid-specific porin (phaK), outer-membrane porin E (oprE), outer-membrane porin D (oprQ), outer membrane protein assembly factor (bamA-II), outer membrane protein assembly factor (bamA-I), outer membrane ferric citrate porin (fecA), OmpA family protein (PP_4198), LPS-assembly protein LptD (IptD), lipid A 3-O-deacylase (pagL-I), glycine-glutamate dipeptide porin (opdP), hypothetical protein (PP_4115), ferrioxamine receptor (PP_0160), ferrichrome-iron receptor (PP_4755), ferric siderophore receptor (PP_3330), ferric siderophore receptor (PP_3325), aromatic compound-specific porin (PP_3656), ferric siderophore receptor (PP_0535), alginate production protein AlgE (algE), alginate biosynthesis protein AlgK (algK), thioredoxin (PSF113_RS59500), thioredoxin (PSF113_RS59220), thioredoxin reductase (PSF113_RS41395), stringent starvation protein A (PSF113_RS54790), peroxidase (PSF113_RS53615), peroxiredoxin (PSF113_RS39210), glutathione peroxidase (PSF113_RS55240), glutathione peroxidase (PSF113_RS39005), catalase (PSF113_RS52605), catalase (PSF113_RS57160), alkyl hydroperoxide reductase subunit F (PSF113_RS42115), secretion protein HylD (PSF113_RS36900), RND transporter MFP subunit (PSF113_RS54645), RND efflux transporter (PSF113_RS35960), multidrug transporter MatE (PSF113_RS50665), multidrug efflux RND transporter permease subunit (PSF113_RS44170), multidrug ABC transporter substrate-binding protein (PSF113_RS50525), acriflavine resistance protein B (PSF113_RS43745), MFS transporter (PSF113_RS53065), MFS transporter (PSF113_RS50550), 4-hydroxybenzoate transporter (PSF113_RS37090), MFS transporter (PSF113_RS38620), TonB-dependent receptor (PSF113_RS57875), protein RlpB (PSF113_RS56535), protein FecA (PSF113_RS55030), porin (PSF113_RS39290), porin (PSF113_RS36845), murein transglycosylase (PSF113_RS54350), membrane protein (PSF113_RS55445), membrane protein (PSF113_RS54915), membrane protein (PSF113_RS40895), maltoporin (PSF113_RS35615), or LPS-assembly protein LptD (PSF113_RS57585).

56. The method of claim 54, wherein the identified genetic element induced upon exposure of the microorganism strain to NAFC or NA is: short-chain oxidoreductase (PP_2789), putative oxidoreductase (PP_0256), paraquat-inducible protein A (PP_0598), p-nitrobenzoate reductase NfnB (PP_3657), oxidoreductase (PP_4020), paraquat-inducible protein A (PP_5745), coniferyl-aldehyde dehydrogenase (calB), dihydroflavonol-4-reductase (PP_2986), short-chain oxidoreductase (PP_1817), dehydrogenase (PP_1661), FMN-dependent NADH-azoreductase (azoR2), 2-carboxybenzaldehyde reductase (yajO), ring-cleaving dioxygenase (PP_3328), 2-alkenal reductase (PSF113_4895), aldo-keto reductase (PSF113_4805), dehydrogenase (PSF113_0191), dehydrogenase (PSF113_4571), FAD-binding oxidoreductase (PSF113_3070), FAD-linked oxidoreductase (PSF113_3227), FMN-dependent NADH-azoreductase (PSF113_1483), Flavin-dependent oxidoreductase (PSF113_0134), dehydrogenase (PSF113_2405), dehydrogenase (PSF113_4207), NAD(P)-dependent oxidoreductase (PSF113_5330), acyl-CoA thioesterase (PSF113_5500), or OHCU decarboxylase (PSF113_4335).

57. The method of claim 49, wherein identifying the genetic element comprises identifying a genetic element related to degradation of NAFC or NA, wherein the method comprises:
overlaying the gene expression profile onto a pathway-genome database to identify an enzyme expressing the DEG in the gene expression profile and determining a substrate relating to the enzyme, and
identifying the enzyme as related to degradation of NAFC or NA if the substrate matches the molecular formula of the NAFC or NA compound.

58. The method of claim 57, further comprising determining a reaction and an associated pathway of the substrate, and
calculating a score for each reaction within the pathway based on the RNA expression levels of the DEG to determine upregulation of the enzyme and the associated pathway in response to degradation of NAFC or NA, and
identifying additional enzymes, inputs, and terminal catabolites of the associated pathway.

59. The method of claim 58, wherein the score is a Reaction Perturbation Score (RPS) or a Pathway Perturbation Score (PPS).

60. The method of claim 59, wherein the identified genetic element induced upon exposure of the microorganism strain to NAFC or NA is: a protocatechuate degradation II pathway, a CFA biosynthesis pathway, a syringate degradation pathway, a benzoate degradation I pathway, a androstenedione degradation pathway, a phenylacetate degradation I pathway, an L-phenylalanine biosynthesis I pathway, a nicotinate degradation pathway, a deethylsimazine degradation pathway, an L-histidine degradation pathway, an allantoin degradation pathway, a taurine degradation pathway, Fatty acid β oxidation I and III pathways, or a Uracil degradation pathway.

61. The method of claim 1, wherein preparing the gene expression profile of the microorganism strain comprises growing two or more microorganism strains separately or in combination with each other: i) in the presence of the toxic organic compound and ii) in the absence of the toxic organic compound, to determine RNA expression levels of the one or more differentially expressed genes to determine co-metabolism related genetic elements.

62. The method of claim 1, wherein the microorganism strain is Pseudomonas fluorescens, Pseudomonas putida, Pseudomonas stutzeri, Pseudomonas sp., Rhodococcus sp., or a combination thereof.

63. A method of generating a gene expression profile of a microorganism isolated from an environment contaminated with toxic organic compounds, comprising:
preparing nucleic acid samples comprising polynucleotides from one or more differentially expressed genes (DEG) from one or more microorganism strains grown: i) in the presence of a toxic organic compound and ii) in the absence of the toxic organic compound;
determining the relative frequency of expressed genes for the one or more microorganism strains;
predicting identities of the one or more DEGs; and
determining a number of reads for the one or more DEGs.

64. The method of claim 63, wherein preparing nucleic acid samples comprising polynucleotides from DEG comprises preparing amplicon cDNA libraries from RNA from DEGs from i) and ii) using Suppression Subtractive Hybridization or similar technology.

65. The method of claim 63, wherein the one or more microorganism strains grown: i) in the presence of the one or more toxic organic compounds and ii) in the absence of the toxic organic compounds, further comprises at least two microorganism strains grown separately from each other or in combination with each other.
